(19) United States
(12) Patent Application Publication
(10) Pub. No.: US 2015/0098993 A1
(43) Pub. Date: Apr. 9, 2015

(54) COMPOSITIONS, PROCESS OF PREPARATION OF SAID COMPOSITIONS AND METHOD OF TREATING INFLAMMATORY DISEASES

(71) Applicants: Shireen Vali, Bangalore (IN); Robinson Vidva, Bangalore (IN); Prashant Ramachandran Nair, Bangalore (IN); Pradeep Fernandes, Saratoga, CA (US); Taher Abbasi, Saratoga, CA (US); Saumya Radhakrishnan, Bangalore (IN)

(72) Inventors: Shireen Vali, Bangalore (IN); Robinson Vidva, Bangalore (IN); Prashant Ramachandran Nair, Bangalore (IN); Pradeep Fernandes, Saratoga, CA (US); Taher Abbasi, Saratoga, CA (US); Saumya Radhakrishnan, Bangalore (IN)

(21) Appl. No.: 14/235,768

(22) PCT Filed: Jul. 27, 2012
(86) PCT No.: PCT/US2012/048739

§ 371 Date: Nov. 25, 2014

(30) Foreign Application Priority Data
Jul. 28, 2011 (IN) 2591/CHE/2011

(51) Int. Cl. A61K 31/506; A61K 31/4178; A61K 31/472; A61K 31/44

(52) U.S. Cl. CPC A61K 31/506; A61K 31/44; A61K 31/4178; A61K 31/472
USPC 424/468; 514/252.18

(57) ABSTRACT

The present invention describes a composition and a kit, each having a plurality of compounds, for use in the treatment of inflammatory joint diseases and chronic inflammatory connective tissue diseases, such as Rheumatoid Arthritis (RA). The invention also relates to a process of obtaining the composition and a method of treating diseases by administration of the compositions.

### CROSS REFERENCE

This application claims priority to Indian Provisional Patent Application No. 2591/CHE/2011, filed on Jul. 28, 2011, which is incorporated by reference herein in its entirety.

### INCORPORATION BY REFERENCE

Every patent, patent application, and non-patent publication recited herein is incorporated by reference in its entirety as if each patent, patent application and non patent publication had been incorporated by reference individually.

### TECHNICAL FIELD

Embodiments of the invention disclosed herein describe compositions and kits, each containing compounds for use in the treatment of inflammatory joint diseases and chronic inflammatory connective tissue diseases, such as Rheumatoid Arthritis (RA). The invention also provides processes for obtaining the compositions and methods of treatment by administration of the compositions.

### BACKGROUND OF THE DISCLOSURE

Joint disease includes any of the diseases or injuries that affect human joints. Arthritis is a generic term for inflammatory joint disease. Inflammation of the joints may cause pain, stiffness, swelling, and some redness of the skin about the joint. Inflammation may be of such nature and severity as to destroy the joint cartilage and underlying bone and cause irreparable deformities, also resulting in loss of mobility (ankylosis). Synovitis occurs when the inflammation is restricted to the lining of the joints. Arthralgias, which is pain in the joints, is a key symptom of Rheumatism that refers to all manners of discomfort of the articular apparatus including joints, bursas, ligaments and tendons. Inflammation of spinal joints is called spondylitis. Bursitis is the inflammation of the lubricating sac or bursa over a joint or between tendons and muscles or bones. Rheumatoid arthritis (RA) and juvenile RA (JRA) are the key diseases in this class of inflammatory joint diseases. The allied arthritic diseases also include psoriatic arthritis, ankylosing spondylitis, infectious arthritis including osteomyelitis, reactive arthritis; intestinal diseases including ulcerative colitis, inflammatory bowel disease (IBD) and the like.

Connective tissue diseases are those with abnormalities in the collagen-containing connective tissues. These are systemic diseases and are also frequently accompanied by joint problems. Systemic lupus erythematosus (SLE) may affect any structure or organ of the body, but has commonality with Rheumatoid arthritis due to the presence of rheumatoid factor. Scleroderma is another collagen disease in which the skin becomes thickened and tight. Rheumatic fever is often classified as a connective tissue disease with transient manifestations of joint issues seen in RA.

Rheumatoid arthritis is one of the most common rheumatic diseases. Features of RA are bilateral tender, warm, swollen joints, joint inflammation, fatigue, occasional fever, long-lasting pain, and stiffness in the morning. In RA, the immune system attacks cells within the joint capsule leading to an autoimmune inflammation called synovitis.

In addition to the local inflammation of the joints, patients in such inflammatory diseases exhibit an increased frequency of cardiovascular disease caused by an associated vasculitis [Bacon, P. A. et al.]. The role of endothelial cell dysfunction is established in the cardiovascular mortality of RA patients. [Bacon P A et al., Int. Rev. Immunol. 2002, 21(1): 1-17]. Endothelial dysfunction causes changes in endothelial-dependent vasodilatation. Anti-tumor necrosis factor-alpha treatment improves endothelial function in patients with rheumatoid arthritis. [Hurlimann, D. et al., Circulation 2002; 106(17): 2184-2187].

Medications commonly used to treat such diseases provide relief from pain and inflammation. Reduction of pain, swelling, and inflammation is reached by treatment with analgesics (e.g. acetaminophen) and Non-Steroidal Anti-Inflammatory Drugs (NSAIDs, e.g. ibuprofen, celecoxib and rofecoxib). To alter the course of the disease, Disease-Modifying Anti-Rheumatic Drugs (DMARDs) are used (e.g. gold (Myochrysine), antimalarials (Plaquenil), penicillamine (Depen)). Corticosteroids such as prednisone and methylprednisolone are also used because of their anti-inflammatory and immunosuppressive effects.

Several types of drugs currently utilized to treat patients with rheumatoid arthritis include analgesics, corticosteroids, uric acid-lowering drugs, immunosuppressive drugs, non-steroidal anti-inflammatory drugs (“NSAIDs”), and disease-modifying antirheumatic drugs (“DMARDs”).

NSAIDs and DMARDs are the most commonly prescribed drugs. NSAIDs are usually the first drugs prescribed and the most commonly used. NSAIDs have a number of serious side effects, but compared to other alternatives are generally well-tolerated by patients at least on an acute basis. DMARDs such as gold and penicillamine are used in patients with more advanced disease and have a higher incidence of toxicity.

Although NSAIDs are efficacious in reducing pain, they have little or no effect on the underlying disease and therefore cannot prevent progression of joint destruction or organ damage. The effects of NSAIDs are relatively rapid, occurring over a period of a few hours. Once the drug is stopped, however, the benefits of its use rapidly fade. There are a number of side effects associated with use of NSAIDs and they are usually dose related. Even with over-the-counter NSAIDs, problematic side effects which include the following: gastrointestinal tract irritation (including ulcers), skin reactions and rashes, increases in blood coagulation time, hepato-cellular toxicity, and impaired renal function. Aspirin, a commonly prescribed NSAID for RA patients, can induce other problems like hypersensitivity responses, tinnitus, and with overdoses may precipitate central nervous system disorders including coma.

Unlike NSAIDs, the DMARDs are thought to have some effect on altering the progression of RA. In general, DMARDs are employed prior to destructive changes in bones or joints. DMARDs include anti-malarial drugs, gold compounds, penicillamine, and sulfasalazine and newer biologics. Further, in contrast to NSAIDs, DMARDs are slower acting and may take weeks or months for benefits of the drug to be noted. Because of this delayed action, some patients prematurely quit the drug because of the perception that the drug is not working. At the proper dosage and with continuous use, a significant reduction in the symptoms of RA may occur in some patients. In some instances, complete remission of RA may also occur. Generally, in an average RA patient, DMARDs are only somewhat effective in at least moderate suppression of symptoms. Unfortunately, some patients do not respond and have had continued active and progressive disease despite taking such drugs. On the contrary, if a particular patient experiences a clinical benefit but discontinues the DMARD the symptoms of the disease are likely to return gradually. Due to the toxicity of DMARDs, patients receiving such medications need to be careful and frequently be re-evaluated by their physicians. All of the DMARDs have significant side effects and include the following: retinal toxicity with the anti-malarial drugs, dermatitis or other skin rashes, nausea, diarrhea and various types of anemia.

Thus, the treatment options for patients with inflammatory joint diseases are limited, particularly so in the case of drugs that can have some effect on altering the progression of such diseases, as opposed to treating symptoms. Since RA is a heterogeneous disease, the patient responses to standard treatments are variable.

Most recent clinical trials of newer DMARDs alone and in combination with methotrexate have shown that ACR50 response—which includes reducing the signs and symptoms of disease by 50%, according to criteria established by the American College of Rheumatology (ACR)—was achieved in less than two-thirds of the patients.

Typically, clinicians have reserved biologics for those patients with severe disease who have failed other therapies. However, the emerging body of evidence suggests that practitioners should be moving toward treating early disease with these biologics.

### SUMMARY OF THE INVENTION

In some embodiments, the invention provides a composition comprising: a) two of: i) an inhibitor of one of colony stimulating factor, platelet derived growth factor, T-cell response, and B-cell response pathways; ii) an inhibitor of phosphodiesterase 4; and iii) an inhibitor associated with angiotensin; and b) a pharmaceutically-acceptable excipient, wherein the composition is a unit dosage form.

In some embodiments, the invention provides a kit comprising: a) two of: i) an inhibitor of one of colony stimulating factor, platelet derived growth factor, T-cell response, and B-cell response pathways; ii) an inhibitor of phosphodiesterase 4; and iii) an inhibitor associated with angiotensin; and b) written instructions on use of the inhibitors.

In some embodiments, the invention provides a method for treating an inflammatory disease in a subject in need or want of relief thereof, the method comprising administering to the subject two of: i) a therapeutically-effective amount of an inhibitor of one of colony stimulating factor, platelet derived growth factor, T-cell response, and B-cell response pathways; ii) a therapeutically-effective amount of an inhibitor of phosphodiesterase 4; and iii) a therapeutically-effective amount of an inhibitor associated with angiotensin.

### BRIEF DESCRIPTION OF THE FIGURES

FIG. 1 illustrates pathways associated with compounds disclosed herein.

FIG. 2 illustrates biochemical targets associated with drugs of the present disclosure.

FIG. 3 illustrates the efficacy of individual drugs CW299, CW304, and CW330, and the combination thereof in terms of ACR Score in TNF responders.

FIG. 4 illustrates the efficacy of individual drugs CW299, CW304, and CW330 and the combination thereof in terms of ACR score in a TNF-resistive system (anti-TNF non-responders).

FIG. 5 illustrates the comparison of the efficacy of combinations of the compounds CW299, CW304, and CW330 in different ratios on clinical parameters of swollen joints, tender joints, CRP, and pain in TNF responders with Etanercept (ENBREL®).

FIG. 6 illustrates a comparison of the efficacy of combinations of the compounds CW299, CW304, and CW330 in different ratios based on clinical parameters of swollen joints, tender joints, CRP, and pain in a TNF non-responder system with Etanercept (ENBREL®).

FIG. 7 illustrates efficacy data of the three individual drugs and the combination thereof for the cytokine biomarker TNF-α (tumor necrosis factor alpha) in TNF responders.

FIG. 8 illustrates efficacy data of the three individual drugs and the combination thereof for the cytokine biomarker IL6 (Interleukin 6) in TNF responders.

FIG. 9 illustrates efficacy data of the three individual drugs and the combination thereof for the chemokine biomarker CCL2.

FIG. 10 illustrates efficacy data of the three individual drugs and the combination thereof for the cytokine biomarker TNF-α (tumor necrosis factor alpha) in TNF non-responders.

FIG. 11 illustrates efficacy data of the three individual drugs and the combination thereof for the cytokine biomarker IL6 (Interleukin 6) in TNF non-responders.

FIG. 12 illustrates efficacy data of the three individual drugs and the combination thereof for the chemokine biomarker CCL2 (otherwise called as MCP 1—monocyte chemotactic protein 1) in TNF non-responders.

FIG. 13 illustrates the efficacy of individual drugs CW299, CW304, CW330 and the combination thereof across clinical parameters of swollen joints, tender joints, CRP, and pain in TNF responders.

FIG. 14 illustrates the efficacy of individual drugs CW299, CW304, CW330 and the combination thereof across clinical parameters of swollen joints, tender joints, CRP, and pain in TNF responders.

FIG. 15 illustrates the efficacy of individual drugs CW299, CW304, CW330 and the combination thereof across clinical parameters of swollen joints, tender joints, CRP, and pain in TNF responders.

FIG. 16 illustrates the efficacy of individual drugs CW299, CW304, CW330 and the combination thereof across clinical parameters of swollen joints, tender joints, CRP, and Pain in TNF responders.

FIG. 17 illustrates the efficacy of individual drugs CW299, CW304, CW330 and the combination thereof across clinical parameters of swollen joints, tender joints, CRP, and pain in TNF-resistant (anti-TNF nonresponsive) system.

FIG. 18 illustrates the efficacy of individual drugs CW299, CW304, CW330 and the combination thereof across clinical parameters of swollen joints, tender joints, CRP, and pain in TNF-resistant (anti-TNF nonresponsive) system.

FIG. 19 illustrates the efficacy of individual drugs CW299, CW304, CW330 and the combination thereof across clinical parameters of swollen joints, tender joints, CRP and pain in TNF-resistant (anti-TNF nonresponsive) system.

FIG. 20 illustrates the efficacy of individual drugs CW299, CW304, CW330 and the combination thereof across clinical parameters of swollen joints, tender joints, CRP, and pain in TNF-resistant (anti-TNF nonresponsive) system.

FIG. 21 illustrates efficacy data of individual drugs CW299 and CW330 and the combination thereof in terms of ACR Score in TNF responders.

FIG. 22 illustrates efficacy the individual drugs CW299 and CW330 and the combination thereof in terms of ACR Score in a TNF-resistive system (anti-TNF non responders).

FIG. 23 illustrates the comparison of the efficacy of combinations (CW299 and CW330) of the compounds on clinical parameters of swollen joints, tender joints, CRP, and pain in TNF responders with Etanercept (ENBREL®).

FIG. 24 illustrates the comparison of the efficacy of combinations (CW299 and CW330) of the compounds on clinical parameters of swollen joints, tender joints, CRP, and pain in a TNF-resistive system (anti-TNF non responders) system with Etanercept (ENBREL®).

FIG. 25 compares the efficacy data of individual drugs and combinations thereof (CW299 and CW330) for the cytokine biomarker TNF-α (tumor necrosis factor alpha) in TNF responders.

FIG. 26 compares the efficacy data of individual drugs and combinations thereof (CW299 and CW330) for the cytokine biomarker IL6 (Interleukin 6) in TNF responders.

FIG. 27 compares the efficacy data of individual drugs and combinations thereof (CW299 and CW330) for the chemokine biomarker CCL2 in TNF responders.

FIG. 28 compares the efficacy data of individual drugs and combinations thereof (CW299 and CW330) for the cytokine biomarker TNF-α (tumor necrosis factor alpha) in TNF resistive system (anti-TNF non responders).

FIG. 29 compares the efficacy data of individual drugs and combinations thereof (CW299 and CW330) for the cytokine biomarker IL6 (Interleukin 6) in a TNF-resistive system (anti-TNF non responders).

FIG. 30 compares the efficacy data of individual drugs and combinations thereof (CW299 and CW330) for the chemokine biomarker CCL2 in a TNF-resistive system (anti-TNF non responders).

FIG. 31 compares the efficacy of individual drugs CW299, CW330 and combinations thereof across clinical parameters of swollen joints, tender joints, CRP, and pain in TNF responders.

FIG. 32 compares the efficacy of individual drugs CW299, CW330 and combinations thereof across clinical parameters of swollen joints, tender joints, CRP and pain, in TNF responders.

FIG. 33 compares the efficacy of individual drugs CW299 and CW330 and combinations thereof across clinical parameters of swollen joints, tender joints, CRP, and pain in TNF resistant (anti-TNF nonresponsive) system.

FIG. 34 compares the efficacy of individual drugs CW299 and CW330 and combinations thereof across clinical parameters of swollen joints, tender joints, CRP, and pain in TNF resistant (anti-TNF nonresponsive) system.

FIG. 35 illustrates efficacy data of the individual drugs CW299 and CW304 and combination thereof in terms of ACR Score in TNF responders.

FIG. 36 illustrates efficacy data of the individual drugs CW299 and CW304 and combination thereof in terms of ACR Score in a TNF-resistive system (anti-TNF non responders).

FIG. 37 illustrates the comparison of the efficacy of combinations (CW299 and CW304) of the compounds on clinical parameters of swollen joints, tender joints, CRP, and pain in TNF responders with Etanercept (ENBREL®).

FIG. 38 illustrates the comparison of the efficacy of combinations (CW299 and CW304) of the compounds on clinical parameters of swollen joints, tender joints, CRP, and pain in a TNF-resistive system (anti-TNF non responders) system with Etanercept (ENBREL®).

FIG. 39 compares the efficacy data of individual drugs and combinations thereof (CW299 and CW304) for the cytokine biomarker TNF-α (tumor necrosis factor alpha) in TNF responders.

FIG. 40 compares the efficacy data of individual drugs and combinations thereof (CW299 and CW304) for the cytokine biomarker IL6 (Interleukin 6) in TNF responders.

FIG. 41 compares the efficacy data of individual drugs and combinations thereof (CW299 and CW304) for the chemokine biomarker CCL2 in TNF responders.

FIG. 42 compares the efficacy data of individual drugs and combinations thereof (CW299 and CW304) for the cytokine biomarker TNF-α (tumor necrosis factor alpha) in a TNF resistive system (anti-TNF non-responders).

FIG. 43 compares the efficacy data of individual drugs and combinations thereof (CW299 and CW304) for the cytokine biomarker IL6 (Interleukin 6) in a TNF-resistive system (anti-TNF non-responders).

FIG. 44 compares the efficacy data of individual drugs and combinations thereof (CW299 and CW304) for the chemokine biomarker CCL2 in a TNF-resistive system (anti-TNF non-responders).

FIG. 45 compares the efficacy of individual drugs CW299 and CW304 and combinations thereof across clinical parameters of swollen joints, tender joints, CRP, and pain in TNF responders.

FIG. 46 compares the efficacy of individual drugs CW299 and CW304 and combinations thereof across clinical parameters of swollen joints, tender joints, CRP, and pain, in TNF responders.

FIG. 47 compares the efficacy of individual drugs CW299 and CW304 and combinations thereof across clinical parameters of swollen joints, tender joints, CRP, and pain in TNF-resistant (anti-TNF nonresponsive) system.

FIG. 48 compares the efficacy of the individual drugs CW304 and CW330 and combinations thereof in terms of ACR Score in TNF responders.

FIG. 49 compares the efficacy of the individual drugs CW304 and CW330 and combinations thereof in terms of ACR Score in a TNF-resistive system (anti-TNF non-responders).

FIG. 50 compares the efficacy of the combination of CW304 and CW330 on measurable clinical parameters such as swollen joints, tender joints, CRP, and pain in TNF responders with Etanercept (ENBREL®).

FIG. 51 compares the efficacy of the combination of CW304 and CW330 on measurable clinical parameters including swollen joints, tender joints, CRP, and pain in a TNF-resistive system (anti-TNF non-responders) system with Etanercept (ENBREL®).

FIG. 52 compares the efficacy of the individual drugs CW304 and CW330 and combinations thereof for the cytokine biomarker TNF-α (tumor necrosis factor alpha) in TNF responders.

FIG. 53 compares the efficacy of the individual drugs CW304 and CW330 and combinations thereof for the cytokine biomarker IL6 (Interleukin 6) in TNF responders.

FIG. 54 compares the efficacy of the individual drugs CW304 and CW330 and combinations thereof for the chemokine biomarker CCL2 in TNF responders.

FIG. 55 compares the efficacy of the individual drugs CW304 and CW330 and combinations thereof for the cytokine biomarker TNF-α (tumor necrosis factor alpha) in a TNF-resistive system (anti-TNF non-responders).

FIG. 56 compares the efficacy of the individual drugs CW304 and CW330 and combinations thereof for the cytokine biomarker IL6 (Interleukin 6) in a TNF-resistive system (anti-TNF non-responders).

FIG. 57 compares the efficacy of the individual drugs CW304 and CW330 and combinations thereof for the chemokine biomarker CCL2 in a TNF-resistive system (anti-TNF non-responders).

FIG. 58 compares the efficacy of the individual drugs CW304 and CW330 and combinations thereof across measurable clinical parameters including swollen joints, tender joints, CRP, and pain, in TNF responders.

FIG. 59 compares the efficacy of the individual drugs CW304 and CW330 and combinations thereof across measurable clinical parameters including swollen joints, tender joints, CRP, and pain in TNF responders.

FIG. 60 compares the efficacy of individual drugs CW304, CW330 and combinations thereof across clinical parameters of swollen joints, tender joints, CRP, and pain in a TNF-resistant (anti-TNF nonresponsive) system.

FIG. 61 compares the efficacy of individual drugs CW304, CW330 and combinations thereof across clinical parameters of swollen joints, tender joints, CRP, and pain in TNF-resistant (anti-TNF nonresponsive) system.

FIG. 62 illustrates examples of pathways associated with the activity of CW299.

FIG. 63 illustrates biochemical targets associated with CW299.

FIG. 64 illustrates predictive data for the efficacy of CW299.

FIG. 65 illustrates experimental data for the efficacy of imatinib mesylate.

FIG. 66 illustrates predictive data for the activity of CW299.

FIG. 67 illustrates experimental data for the activity of imatinib.

FIG. 68 illustrates predictive data for the activity of CW299 for CRP.

FIG. 69 illustrates experimental data for the activity of imatinib for CRP.

FIG. 70 illustrates predictive data for the activity of CW299 for pain.

FIG. 71 illustrates experimental data for the activity of imatinib for pain.

FIG. 72 illustrates examples of pathways associated with the activity of CW304.

FIG. 73 illustrates biochemical targets associated with CW304.

FIG. 74 illustrates predictive data for the efficacy of CW304.

FIG. 75 illustrates experimental data for the efficacy of apremilast.

FIG. 76 illustrates predictive data for the activity of CW304 for TNFα.

FIG. 77 illustrates experimental data for the activity of apremilast for TNFα.

FIG. 78 illustrates examples of pathways associated with the activity of CW330.

FIG. 79 illustrates biochemical targets associated with CW330.

FIG. 80 illustrates predictive data for the efficacy of CW330.

FIG. 81 illustrates experimental data for the efficacy of olmesartan.

FIG. 82 illustrates predictive data for the efficacy of CW330.

FIG. 83 illustrates experimental data for the efficacy of quinapril.

FIG. 84 illustrates predictive data for the activity of CW330 for TNFα.

FIG. 85 illustrates experimental data for the activity of quinapril for TNFα.

FIG. 86 illustrates examples of pathways associated with the activity of CW299 and CW304.

FIG. 87 illustrates the comparison of the efficacy of individual drugs CW299, CW304, and combinations thereof based on N-ACR score.

FIG. 88 illustrates the comparison of the efficacy of individual drugs CW299 and CW304, and combinations thereof at different dosages on clinical parameters of swollen joints, tender joints, CRP, and pain.

FIG. 89 illustrates the comparison of the efficacy of individual drugs CW299, CW304, and combinations thereof based on TNFα.

FIG. 90 illustrates the comparison of the efficacy of individual drugs CW299, CW304, and combinations thereof based on IL-17.

FIG. 91 illustrates examples of pathways associated with the activity of CW299 and CW330.

FIG. 92 illustrates the comparison of the efficacy of individual drugs CW299, CW330, and combinations thereof based on N-ACR score.

FIG. 93 illustrates the comparison of the efficacy of individual drugs CW299 and CW330, and combinations thereof at different dosages on clinical parameters of swollen joints, tender joints, CRP, and pain.

FIG. 94 illustrates the comparison of the efficacy of individual drugs CW299, CW330, and combinations thereof based on TNFα.

FIG. 95 illustrates the comparison of the efficacy of individual drugs CW299, CW330, and combinations thereof based on IL-17.

FIG. 96 illustrates examples of pathways associated with the activity of CW304 and CW330.

FIG. 97 illustrates the comparison of the efficacy of individual drugs CW304, CW330, and combinations thereof based on N-ACR score.

FIG. 98 illustrates the comparison of the efficacy of individual drugs CW304 and CW330, and combinations thereof for different dosages on clinical parameters of swollen joints, tender joints, CRP, and pain.

FIG. 99 illustrates the comparison of the efficacy of individual drugs CW304, CW330, and combinations thereof based on TNFα.

FIG. 100 illustrates the comparison of the efficacy of individual drugs CW304, CW330, and combinations thereof based on IL-17.

FIG. 101 illustrates examples of pathways associated with the activity of CW299, CW304 and CW330.

FIG. 102 illustrates the comparison of the efficacy of individual drugs CW299, CW304, CW330 and two-drug (CW299304, CW299330 and CW304330) and three-drug (CW299204220) combinations thereof based on N-ACR score.

FIG. 103 illustrates the comparison of the efficacy of individual drugs CW299, CW304, CW330 and two-drug (CW299304, CW299330 and CW304330) and three-drug (CW299204220) combinations thereof on clinical parameters of swollen joints, tender joints, CRP, and pain.

FIG. 104 illustrates the comparison of the efficacy of individual drugs CW299, CW304, CW330 and two-drug (CW299304, CW299330 and CW304330) and three-drug (CW299204220) combinations thereof based on TNFα.

FIG. 105 illustrates the comparison of the efficacy of individual drugs CW299, CW304, CW330 and two-drug (CW299304, CW299330 and CW304330) and three-drug (CW299204220) combinations thereof based on IL-17.

### DETAILED DESCRIPTION OF DISCLOSURE

In some embodiments, the invention provides two-drug and three-drug combinations, which provide multi-targeted combination therapeutic approach to suppress and cure symptoms associated with Rheumatoid Arthritis. The drug combinations were validated using virtual co-culture computational simulations as described herein. Simulations performed with each individual drug provided more than 80% marker trend correlation to the published experimental literature described and incorporated herein.

The two-drug and three-drug combinations provide synergistic efficacy on the end-point markers, while dosing as low as 1/20 of the recommended therapeutic dose of the drug in humans. Using a lower dose of the individual drug also provides an advantage in terms of minimizing the intensity of side-effects or toxicities associated with the drugs. Also, the drug combination works by inhibiting multiple targets minimally, so that an amplified effect is observed on all of the primary end-point markers and at the same time ensuring that all the targets have primary response ability, so as to negate the possibility of immune suppression and secondary infections.

Use of smaller doses of individual drugs also lowers the cost of manufacture and formulation, providing an improved effect at lower price to the subject. Smaller doses also mitigate against wasteful administration of a drug to a physiological system that has been saturated or has reached a peak therapeutic response from smaller, synergistic doses.

In some embodiments, the invention provides a composition comprising: a) two or three of: i) an inhibitor of one of colony stimulating factor, platelet derived growth factor, T-cell response and B-cell response pathways; ii) an inhibitor of phosphodiesterase 4; and iii) an inhibitor associated with angiotensin; and b) optionally a pharmaceutically-acceptable excipient. The inhibitor associated with angiotensin can be, for example, an inhibitor of an angiotensin II AT1 receptor or an inhibitor of angiotensin-converting enzyme.

In some embodiments, the invention provides a kit comprising: a) two or three of: i) an inhibitor of one of colony stimulating factor, platelet derived growth factor, T-cell response and B-cell response pathways; ii) an inhibitor of phosphodiesterase 4; and iii) an inhibitor associated with angiotensin; and b) optionally a pharmaceutically-acceptable excipient, wherein the kit comprises one or a plurality of dosage forms. The inhibitor associated with angiotensin can be, for example, an inhibitor of an angiotensin II AT1 receptor or an inhibitor of angiotensin-converting enzyme.

The invention disclosed herein provides combinations of three classes of drugs, which exhibit converging antagonistic effects on major pro-inflammatory transcription factors through different mechanisms of action. Inhibition of factors such as NFkB and API causes a systemic reduction in the pro-inflammatory cytokines and chemokines that are involved in disease physiology and progression. In addition to targeting multiple pathways and nodes, the drug combinations target multiple cell types. In some embodiments, the inhibition of multiple pathways in multiple cell types provides a systemic effect, even at low drug concentrations.

The drug combinations provided herein are effective in TNF resistive systems (anti-TNF non-responders), because the three drug compounds affect diverse (TNF independent) strategic signaling points distributed across three distinct pathways in the relevant cell systems. This phenomenon allows even minor inhibitory effects from each strategic point to produce an enhanced inhibitory effect upon convergence of the minor effects, thereby amplifying the effect on the pool of biomarkers secreted from the various cell types present in the synovium.

The CW299 class of drugs can inhibit multiple cell types, including, for example, Macrophages, B-Lymphocytes, T-Lymphocytes, Mast cells, Synovial Fibroblasts, Endothelial cells, Chondrocytes, Dendritic cells, Osteoclasts and Osteoblasts. CW299 can function by inhibiting one or more of macrophage colony stimulating factor (CSF1), platelet derived growth factor (PDGFR), T-cell response (TCR) and B-cell response (BCR) pathways. In some embodiments, CW299 inhibits all of colony stimulating factor (CSF1), platelet derived growth factor (PDGFR), T cell response (TCR) and B-cell response (BCR) pathways. In some embodiments, CW299 is an inhibitor of colony stimulating factor (CSF1), platelet derived growth factor (PDGFR), T-cell response (TCR) and B-cell response (BCR) pathways.

The CW304 class of drugs can inhibit the enzyme phosphodiesterase 4. In an embodiment, the CW304 class of drugs is represented by roflumilast. Roflumilast is a potent and highly selective inhibitor of the enzyme PDE4. PDE4 is expressed in inflammatory cells such as neutrophils and macrophages. It degrades the second messenger cyclic AMP (cAMP). High levels of intracellular cAMP reduce the activity of inflammatory cells; therefore roflumilast increases intracellular cAMP levels and reduces inflammatory cell activity.

The CW330 class of drugs can inhibit Angiotensin II Type 1 Receptors or Angiotensin-converting enzyme. In an embodiment, the CW330 class of drugs is represented by olmesartan or quinaprilat. Angiotensin II is formed from angiotensin I in a reaction catalyzed by angiotensin converting enzyme (ACE). Angiotensin II is the principal pressor agent of the renin-angiotensin system. Olmesartan blocks the vasoconstrictor effects of angiotensin II by selectively blocking the binding of angiotensin II to the AT1 receptor. It has more than a 12,500-fold greater affinity for the AT1 receptor than for the AT2 receptor. Quinaprilat compound also competes with ATI for binding to ACE and inhibits and enzymatic proteolysis of ATI to ATII. Decreasing ATII levels in the body decreases blood pressure by inhibiting the pressor effects of ATII.

As used herein, the term, “CW299304330,” refers to a combination of any CW299 compound, any CW304 compound, and any CW330 compound in any amount, ratio, concentration, or order thereof.

As used herein, the term, “CW299304,” refers to a combination of any CW299 compound, and any CW304 compound in any amount, ratio, concentration, or order thereof.

As used herein, the term, “CW299330,” refers to a combination of any CW299 compound and any CW330 compound in any amount, ratio, concentration, or order thereof.

As used herein, the term, “CW304330,” refers to a combination of any CW304 compound and any CW330 compound in any amount, ratio, concentration, or order thereof.

Non-limiting examples of CW299 include: a) Imatinib or a pharmaceutically-acceptable salt thereof, such as Imatinib Mesylate; b) Sti-571 or a pharmaceutically-acceptable salt thereof; c) Nilotinib or a pharmaceutically acceptable salt thereof; d) Dasatinib or a pharmaceutically-acceptable salt thereof, e) Sunitinib or a pharmaceutically-acceptable salt thereof, such as Sunitinib malate; f) Masitinib or a pharmaceutically-acceptable salt thereof, such as masitinib mesylate; g) Bosutinib or a pharmaceutically-acceptable salt thereof; h) Ponatinib or a pharmaceutically-acceptable salt thereof, i) Bafetinib or a pharmaceutically-acceptable salt thereof, j) CYC 10268 or a pharmaceutically-acceptable salt thereof, and any combination thereof.

Non-limiting examples of CW304 include: a) Roflumilast or a pharmaceutically-acceptable salt thereof, b) Daxas or a pharmaceutically-acceptable salt thereof, c) Rolipram or a pharmaceutically-acceptable salt thereof, d) Ibudilast/AV-411/MN-166 or a pharmaceutically-acceptable salt thereof, e) Apremilast or a pharmaceutically-acceptable salt thereof, and any combination thereof.

Non-limiting examples of CW330 include: a) Candesartan/Atacand or a pharmaceutically-acceptable salt thereof; b) Eprosartan/Teveten or a pharmaceutically-acceptable salt thereof; c) Forasartan or a pharmaceutically-acceptable salt thereof; d) Irbesartan/Avapro or a pharmaceutically-acceptable salt thereof; e) Losartan/Cozaar or a pharmaceutically-acceptable salt thereof; f) Olmesartan/Benicar or a pharmaceutically-acceptable salt thereof; g) Saprisartan or a pharmaceutically-acceptable salt thereof; h) Tasosartan or a pharmaceutically-acceptable salt thereof; i) Telmisartan/Micardis or a pharmaceutically-acceptable salt thereof; j) Valsartan/Diovan or a pharmaceutically-acceptable salt thereof; k) Azilsartan/Edarbi or a pharmaceutically-acceptable salt thereof; l) Fosinopril/Monopril or a pharmaceutically-acceptable salt thereof; m) Benazepril/Lotensin or a pharmaceutically-acceptable salt thereof; n) Candoxatril/Candoxatrilat or a pharmaceutically-acceptable salt thereof; o) Captopril/Capoten or a pharmaceutically-acceptable salt thereof; p) Cilazapril or a pharmaceutically-acceptable salt thereof; q) Enalapril/Vasotec or a pharmaceutically-acceptable salt thereof; r) Lisinopril/Prinivil or a pharmaceutically-acceptable salt thereof; s) Moexipril/Univasc or a pharmaceutically-acceptable salt thereof; t) Perindopril/Aceon or a pharmaceutically-acceptable salt thereof; u) Quinapril/Accupril or a pharmaceutically-acceptable salt thereof; v) Ramipril/Acovil or a pharmaceutically-acceptable salt thereof; w) Rescinnamine/Anapral or a pharmaceutically-acceptable salt thereof; x) Spirapril/Renormax or a pharmaceutically-acceptable salt thereof; y) Trandolapril/Mavik or a pharmaceutically-acceptable salt thereof, and any combination thereof.

Non-limiting examples of CW299 include the compounds of Table 1.

TABLE 1
CW299 Compound Names.

| | | EXEMPLARY | |
|---|---|---|---|
| COMPOUNDS | SYNONYMS | SALTS | IUPAC NOMENCLATURE |
| Imatinib | | Imatinib Mesylate | N-(4-methyl-3-{[4-(pyridin-3- |
| | | (Imatinib | yl)pyrmidin-2-yl]amino}phenyl)-4- |
| | | Methansulfonate) | [(4-methylpiperazin-1- |
| | | | yl)methyl]benzamide |
| Nilotinib | AMN-107/ | | 4-methyl-N-[3-(4-methyl-1H- |
| | AMN107 | | imidazol-1-yl)-5- |
| | | | (trifluoromethyl)phenyl]-3-{[4- |
| | | | (pyridin-3-yl)pyrimidin-2- |
| | | | yl]amino}benzamide |
| Dasatinib | BMS- | | N-(2-chloro-6-methylphenyl)-2-({6- |
| | 354825 | | [4-(2-hydroxyethyl)piperazin-1-yl]- |
| | | | 2-methylpyrimidin-4-yl}amino)-1,3- |
| | | | thiazole-5-carboxamide |
| Sunitinib | SU11248; | Sunitinib malate | N-[2-(diethylamino)ethyl]-5-{[(3Z)- |
| | Sutent | | 5-fluoro-2-oxo-2,3-dihydro-1H- |
| | | | indol-3-ylidene]methyl}-2,4- |
| | | | dimethyl-1H-pyrrole-3- |
| | | | carboxamide |
| Masitinib | | Masitinib | 4-(4-methylpiperazin-1-ylmethyl)- |
| | | mesylate | N-[4-methyl-3-(4-pyridin-3ylthiazol- |
| | | | 2-ylamino)phenyl]benzamide- |
| | | | mesylate methane sulfonic acid salt, |
| Bosutinib | SKI 606; | | 4-[(2,4-dichloro-5- |
| | SKI-606 | | methoxyphenyl)amino]-6-methoxy- |
| | | | 7-[3-(4-methylpiperazin-1- |
| | | | yl)propoxy]quinoline-3 -carbonitrile |
| Ponatinib | AP24534 | | 3-(imidazo[1,2-b]pyridazin-3- |
| | | | ylethynyl)-4-methyl-N-(4-((4- |
| | | | methylpiperazin-1-yl)methyl)-3- |
| | | | (trifluoromethyl)phenyl)benzamide. |
| Bafetinib | CNS-9; Dual | | Benzamide, N-[3-([4,5′- |
| | Bcr-Abl; Lyn | | bipyrimidin]-2-ylamino)-4- |
| | Tyrosine | | methylphenyl]-4-[[(3S)-3- |
| | Kinase | | (dimethylamino)-1- |
| | Inhibitor | | pyrrolidinyl]methyl]-3- |
| | INNO-406; | | (trifluoromethyl)-benzamide |
| | INNO-406; | | |
| | NS-187 | | |
| CYC10268 | | | 2-methoxy-4-(6-(1- |
| | | | phenylpropylamino)pyrazin-2-yl) |
| | | | phenol |

Chemical structures of the compounds of Table 1 are as follows:

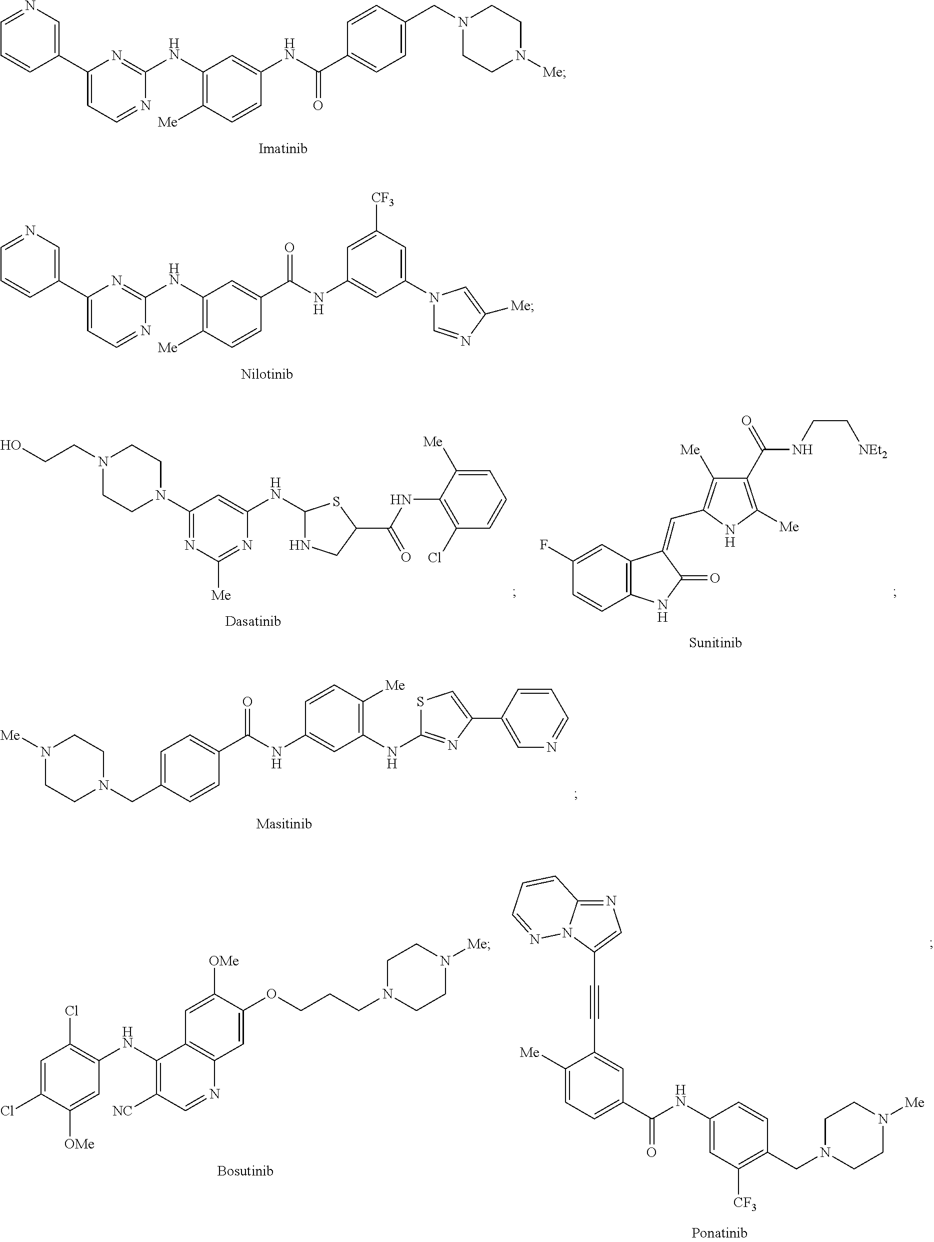

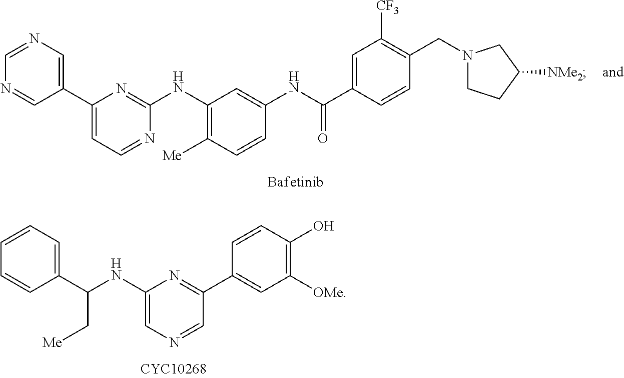 [SMILES: Cc1ccc(NC(=O)c2ccc(CN3CC[C@@H](N(C)C)C3)c(C(F)(F)F)c2)cc1Nc1nccc(-c2cncnc2)n1 ; "and" ; CCC(Nc1cncc(-c2ccc(O)c(OC)c2)n1)c1ccccc1]

In some embodiments, a compound of the invention is a compound of the formula (I):

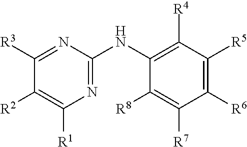

wherein:
R¹ is a cyclic group that is substituted or unsubstituted;
R² and R³ are each independently hydrogen or alkyl;
one or two of the groups R⁴, R⁵, R⁶, R⁷, and R⁸ are each nitro, alkoxy, fluoro substituted alkoxy, or a group of the formula: —N(R⁹)—C(═X)—(Y)ₙ—R¹⁰, wherein:
   R⁹ is hydrogen or alkyl;
   X is oxo, thio, ═NH, ═N(alkyl), ═NOH, or ═NO(alkyl);
   Y is oxygen, NH, or N(alkyl);
   n is 0 or 1; and
   R¹⁰ is an alkyl group or a cyclic group,
and the remaining groups R⁴, R⁵, R⁶, R⁷, and R⁸ are each independently: hydrogen; alkyl that is unsubstituted or substituted by amino, alkyl amino, or dialkyl amino; a heterocycle; acyl; trifluoromethyl; hydroxyl; alkoxyl; amino; alkyl amino; dialkyl amino; amido; carbamato; a carboxylic acid; or an ester group,
   or a pharmaceutically-acceptable salt thereof.

In some embodiments, R is 4-pyrazinyl, 1-methyl-1H pyrrolyl, phenyl; phenyl substituted with amino, alkyl amino, dialkyl amino, or acyl amino; phenyl substituted with aminoalkyl; phenyl substituted with alkyl; 1H-indolyl; 1H-imidazolyl; pyridyl; pyridyl substituted with alkyl; pyridyl N-oxide; or pyridyl N-oxide substituted with alkyl. R² and R³ are each independently hydrogen or alkyl. One or two of the groups R⁴, R⁵, R⁶, R⁷, and R⁸ are each nitro, fluoro-substituted alkoxy, or a group of the formula: —N(R⁹)—C(═X)—(Y)ₙ—R, wherein:
R⁹ is hydrogen or alkyl;
X is oxo, thio, ═NH, ═N(alkyl), ═NOH, or ═NO(alkyl);
Y is oxygen or NH;
n is 0 or 1; and
R¹⁰ is an aliphatic hydrocarbon group having 5-22 carbon atoms; a phenyl or naphthyl group each of which is unsubstituted or substituted by cyano, alkyl, alkoxy, (4-methyl-piperazinyl)-alkyl, trifluoromethyl, hydroxy, alkanoyloxy, halogen, amino, alkyl amino, dialkyl amino, alkanoyl amino, benzoyl amino, carboxy or by alkoxycarbonyl; or phenyl-alkyl wherein the phenyl group is unsubstituted or substituted as indicated above; a cycloalkyl or cycloalkenyl group having up to 30 carbon atoms; a cycloalkyl-alkyl or cycloalkenyl-alkyl group each having up to 30 carbon atoms; a heterocyclic group having 5 or 6 ring members and 1-3 ring heteroatoms selected from nitrogen, oxygen and sulfur, to which heterocyclic group one or two carbocyclic rings may be fused, and the remaining groups R⁴, R⁵, R⁶, R⁷, and R⁸ are each independently: hydrogen; alkyl that is unsubstituted or substituted by amino, alkyl amino, or dialkyl amino; a heterocycle; acyl; trifluoromethyl; hydroxyl; alkoxy; amino; alkyl amino; dialkyl amino; amido; carbamato; a carboxylic acid; or a carboxylic ester.

In some embodiments, R¹ is pyridyl or pyridyl N-oxide; R² and R³ are each hydrogen; R⁴ is hydrogen or alkyl; R⁵ is hydrogen or alkyl; R⁶ is hydrogen; R⁷ is a group of the formula: —N(R⁹)—C(═X)—(Y)ₙ—R¹⁰, wherein:
R⁹ is hydrogen;
X is oxo;
n is 0; and
R¹⁰ is a phenyl group that is unsubstituted or substituted by cyano, alkyl, (4-methyl-piperazinyl)methyl, or halogen; and

R⁸ is hydrogen.

In some embodiments, R¹ is 3-pyridyl; R² and R³ are each hydrogen; R⁴ is hydrogen or methyl; R⁵ is hydrogen or methyl; R⁶ is hydrogen; R⁷ is a group of the formula: —N(R⁹)—C(═X)—(Y)ₙ—R¹⁰, wherein:
R⁹ is hydrogen;
X is oxo;
n is 0; and
R¹⁰ is a phenyl group that is unsubstituted or substituted by (4-methyl-piperazinyl)methyl; and

R⁸ is hydrogen.

In some embodiments, the compound is:

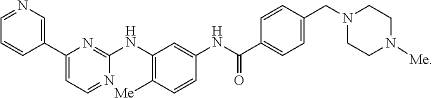 [SMILES: Cc1ccc(NC(=O)c2ccc(CN3CCN(C)CC3)cc2)cc1Nc1nccc(-c2cccnc2)n1]

In some embodiments, the compound is Imatinib. In some embodiments, the pharmaceutically-acceptable salt is a mesylate. In some embodiments, the compound is Imatinib mesylate.

Non-limiting examples of CW304 include the compounds of Table 2.

TABLE 2
CW304 Compound Names.

| | | | |
|---|---|---|---|
| COM- | SYN- | EXEMPLARY | IUPAC |
| POUNDS | ONYMS | SALTS | NOMENCLATURE |

| | | |
|---|---|---|
| Roflumilast | Daxas | 3-(cyclopropylmethoxy)- |
| | | N-(3,5-dichloropyridin- |
| | | 4-yl)-4-(difluorometh- |
| | | oxy)benzamide |
| Rolipram | | (RS)-4-(3-cyclopentyloxy- |
| | | 4-methoxy-phenyl)- |
| | | pyrrolidin-2-one |
| Ibudilast | AV-411; | 2-methyl-1-(2-propan-2-yl- |
| | MN-166 | pyrazolo[1,5-a]pyridin- |
| | | 3-yl)propan-1-one |
| Apremilast | | (S)-N-{2-[1-(3-Ethoxy- |
| | | 4-methoxyphenyl)-2-methane- |
| | | sulfonylethyl]-1,3-dioxo- |
| | | 2,3-dihydro-1H-isoindol-4- |
| | | yl}acetamide |

Chemical structures of the compounds of Table 2 are as follows:

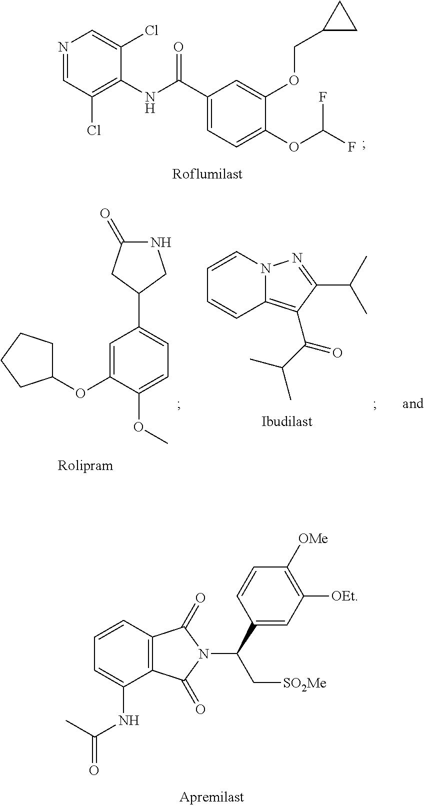

In some embodiments, a compound of the invention is a compound of the formula (II), or an N-oxide thereof:

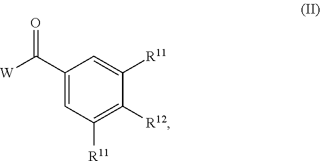

each R¹¹ is independently H, halogen, OH, SH, cyano, nitro, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, acyl, O-acyl, carbamato, a urethane, a carbonate, amino, mono-substituted amino, or di-substituted amino, wherein any of the foregoing is independently unsubstituted or substituted with any number of halogen atoms;
R¹² is H, halogen, OH, SH, cyano, nitro, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, acyl, O-acyl, carbamato, a urethane, a carbonate, amino, mono-substituted amino, or di-substituted amino, wherein any of the foregoing is independently unsubstituted or substituted with any number of halogen atoms;
W is OR¹³, SR¹³, NHR¹³, N(R¹³)₂, wherein each R¹³ is independently alkyl, alkenyl, alkynyl, aryl, aralkyl, heterocyclyl, or heterocyclylalkyl, wherein any of the foregoing is independently unsubstituted or substituted with any number of halogen atoms, or a pharmaceutically-acceptable salt thereof.

In some embodiments, each R¹¹ is independently H, alkoxy, aryloxy, O-heterocyclyl, O-heterocyclylalkyl, O-acyl, carbamato, a urethane, a carbonate, amino, mono-substituted amino, or di-substituted amino, wherein any of the foregoing is independently unsubstituted or substituted with any number of halogen atoms; R¹² is H, alkoxy, aryloxy, O-heterocyclyl, O-heterocyclylalkyl, O-acyl, carbamato, a urethane, a carbonate, amino, mono-substituted amino, or di-substituted amino, wherein any of the foregoing is independently unsubstituted or substituted with any number of halogen atoms; and W is NHR¹³ or N(R¹³)₂, wherein each R¹³ is independently a group of formula (IIA) or formula (IIB)

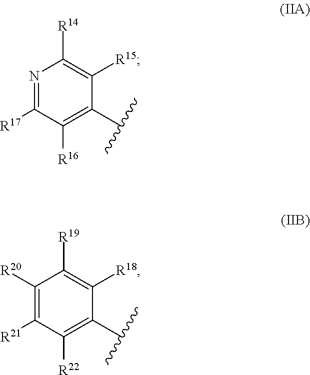

wherein each of R¹⁴, R¹⁵, R¹⁶, R¹⁷, R¹⁸, R¹⁹, R²⁰, R²¹, and R²² is independently H, halogen, OH, SH, cyano, nitro, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, acyl, O-acyl, carbamato, a urethane, a carbonate, amino, mono-substituted amino, or di-substituted amino, or a pharmaceutically-acceptable salt thereof.

In some embodiments, each R¹¹ is independently H, alkoxy, or aryloxy, wherein any of the foregoing is independently unsubstituted or substituted with any number of halogen atoms; R¹² is H, alkoxy, or aryloxy, wherein any of the foregoing is independently unsubstituted or substituted with any number of halogen atoms; W is NHR¹³ or N(R¹³)₂, wherein each R¹³ is independently a group of formula (IIA) or formula (IIB)

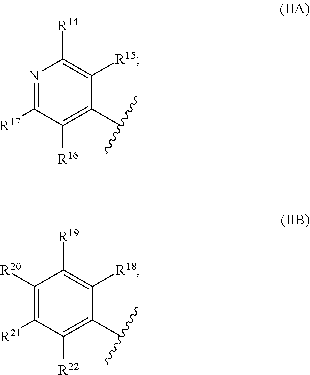

wherein each of R¹⁴, R¹⁵, R¹⁶, R¹⁷, R¹⁸, R¹⁹, R²⁰, R²¹, and R²² is independently H, halogen, OH, SH, cyano, nitro, alkyl, alkoxy, amino, mono-substituted amino, or di-substituted amino, or a pharmaceutically-acceptable salt thereof.

In some embodiments, each R¹¹ is independently H, cycloakyloxy, cycloalkylalkoxy, aralkyloxy, or aryloxy, wherein any of the foregoing is independently unsubstituted or substituted with any number of halogen atoms; R¹² is alkoxy, which is unsubstituted or substituted with any number of halogen atoms; W is NHR¹³, wherein R¹³ is a group of formula (IIA)

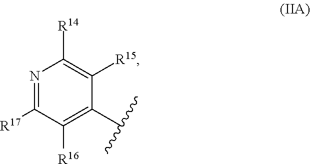

wherein each of R¹⁴, R¹⁵, R¹⁶, and R¹⁷ is independently H, halogen, OH, SH, cyano, nitro, alkyl, alkoxy, amino, mono-substituted amino, or di-substituted amino, or a pharmaceutically-acceptable salt thereof.

In some embodiments: each R¹¹ is independently H or cycloalkylalkoxy; R¹² is alkoxy substituted with any number of fluorine atoms; W is NHR¹³, wherein R¹³ is a group of formula (IIA)

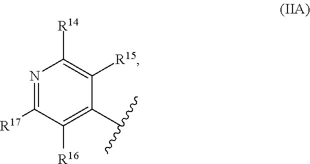

wherein each of R¹⁴, R¹⁵, R¹⁶, and R¹⁷ is independently H or halogen, or a pharmaceutically-acceptable salt thereof.

In some embodiments, the compound is:

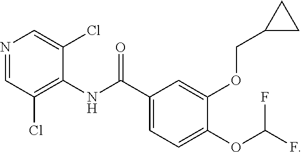 [SMILES: O=C(Nc1c(Cl)cncc1Cl)c1ccc(OC(F)F)c(OCC2CC2)c1]

In some embodiments, the compound is Roflumilast. In some embodiments, the compound is Roflumilast N-oxide.

Non-limiting examples of CW330 include the compounds of Table 3.

TABLE 3
CW330 Compound Names.

| COMPOUNDS | SYNONYMS | IUPAC NOMENCLATURE |
|---|---|---|
| Candesartan | Atacand | 2-ethoxy-1-({4-[2-(2H-1,2,3,4-tetrazol-5- |
| | | yl)phenyl]phenyl}methyl)-1H-1,3-benzodiazole-6- |
| | | carboxylic acid |
| Eprosartan | Teveten | 4-({2-butyl-5-[2-carboxy-2-(thiophen-2-ylmethyl)eth-1- |
| | | en-1-yl]-1H-imidazol-1-yl}methyl)benzoic acid |
| Forasartan | | 5-[(dibutyl-1H-1,2,4-triazol-1-yl)methyl]-2-[2-(2H- |
| | | 1,2,3,4-tetrazol-5-yl)phenyl]pyridine |
| Irbesartan | Avapro | 2-butyl-3-({4-[2-(2H-1,2,3,4-tetrazol-5- |
| | | yl)phenyl]phenyl}methyl)-1,3-diazaspiro[4.4]non-1-en- |
| | | 4-one |
| Losartan | Cozaar | [2-butyl-5-chloro-3-[[4-[2-(2H-tetrazol-5- |
| | | yl)phenyl]phenyl]methyl] |
| | | imidazol-4-yl]methanol |
| Olmesartan | Benicar | (5-methyl-2-oxo-2H-1,3-dioxol-4-yl)methyl 4-(2- |
| | | hydroxypropan-2-yl)-2-propyl-1-({4-[2-(2H-1,2,3,4- |
| | | tetrazol-5-yl)phenyl]phenyl}methyl)-1H-imidazole-5- |
| | | carboxylate |
| Saprisartan | | 1-({3-bromo-2-[2- |
| | | (trifluoromethane)sulfonamidophenyl]-1-benzofuran-5- |
| | | yl}methyl)-4-cyclopropyl-2-ethyl-1H-imidazole-5- |
| | | carboxamide |
| Tasosartan | | 2,4-dimethyl-8-[[4-[2-(2H-tetrazol-5- |
| | | yl)phenyl]phenyl]methyl]-5, |
| | | 6-dihydropyrido[2,3-d]pyrimidin-7-one |
| Telmisartan | Micardis | 2-(4-{[4-methyl-6-(1-methyl-1H-1,3-benzodiazol-2-yl)- |
| | | 2-propyl-1H-1,3-benzodiazol-1- |
| | | yl]methyl}phenyl)benzoic acid |
| Valsartan | Diovan | (S)-3-methyl-2-[N-({[2′-(2H-1,2,3,4-tetrazol-5- |
| | | yl)phenyl]phenyl}methyl) pentanamido]butanoic acid |
| Azilsartan | Edarbi | (5-methyl-2-oxo-1,3-dioxol-4-yl)methyl 2-ethoxy-1-([2′- |
| | | (5-oxo-4,5-dihydro-1,2,4-oxadiazol-3-yl)biphenyl-4- |
| | | yl]methyl)-1H-benzimidazole-7-carboxylate |
| Fosinopril | Monopril | (2S,4S)-4-cyclohexyl-1-(2-{[2-methyl-1-(propanoyloxy) |
| | | propoxy] (4-phenylbutyl) phosphoryl} acetyl) |
| | | pyrrolidine-2-carboxylic acid |
| Benazepril | Lotensin | 2-[(3S)-3-{[(2S)-1-ethoxy-1-oxo-4-phenylbutan-2- |
| | | yl]amino}-2-oxo-2,3,4,5-tetrahydro-1H-1-benzazepin-1- |
| | | yl]acetic acid |
| Candoxatril | Candoxatrilat | 4-[[1-[(2S)-3-(2,3-dihydro-1H-inden-5-yloxy)-2-(2- |
| | | methoxyethoxymethyl)-3-oxopropyl] |
| | | cyclopentanecarbonyl]amino]cyclohexane-1-carboxylic |
| | | acid |
| Captopril | Capoten | (2S)-1-[(2S)-2-methyl-3-sulfanylpropanoyl]pyrrolidine- |
| | | 2-carboxylic acid |
| Cilazapril | | (1S,9S)-9-{[(2S)-1-ethoxy-1-oxo-4-phenylbutan-2- |
| | | yl]amino}-10-oxo-octahydro-1H-pyridazino[1,2- |
| | | a][1,2]diazepine-1-carboxylic acid |
| Enalapril | Vasotec | (2S)-1-[(2S)-2-{[(2R)-1-ethoxy-1-oxo-4-phenylbutan-2- |
| | | yl]amino}propanoyl]pyrrolidine-2-carboxylic acid |
| Lisinopril | Prinivil | (2S)-1-[(2S)-6-amino-2-[[(1S)-1-carboxy-3- |
| | | phenylpropyl]amino]hexanoyl] |
| | | pyrrolidine-2-carboxylic acid |
| Moexipril | Univasc | (3S)-2-[(2S)-2-{[(2S)-1-ethoxy-1-oxo-4-phenylbutan-2- |
| | | yl]amino}propanoyl]-6,7-dimethoxy-1,2,3,4- |
| | | tetrahydroisoquinoline-3-carboxylic acid |
| Perindopril | Aceon | (2S,3aS,7aS)-1-[(2S)-2-{[(2S)-1-ethoxy-1-oxopentan-2- |
| | | yl]amino}propanoyl]-octahydro-1H-indole-2-carboxylic |
| | | acid |
| Quinapril | Accupril | (3S)-2-[(2S)-2-{[(2S)-1-ethoxy-1-oxo-4-phenylbutan-2- |
| | | yl]amino}propanoyl]-1,2,3,4-tetrahydroisoquinoline-3- |
| | | carboxylic acid |
| Ramipril | Acovil | (2S,3aS,6aS)-1-[(2S)-2-{[(2S)-1-ethoxy-1-oxo-4- |
| | | phenylbutan-2-yl]amino}propanoyl]- |
| | | octahydrocyclopenta[b]pyrrole-2-carboxylic acid |
| Rescinnamine | Anapral | methyl (1R,15S,17R,18R,19S,20S)-6,18-dimethoxy-17- |
| | | [(E)-3-(3,4,5-trimethoxyphenyl)prop-2-enoyl]oxy- |
| | | 1,3,11,12,14,15,16,17,18,19,20,21- |
| | | dodecahydroyohimban-19-carboxylate |
| Spirapril | Renormax | (8S)-7-[(2S)-2-{[(2S)-1-ethoxy-1-oxo-4-phenylbutan-2- |
| | | yl]amino}propanoyl]-1,4-dithia-7-azaspiro[4.4]nonane- |
| | | 8-carboxylic acid |
| Trandolapril | Mavik | (2S,3aR,7aS)-1-[(2S)-2-{[(2S)-1-ethoxy-1-oxo-4- |
| | | phenylbutan-2-yl]amino}propanoyl]-octahydro-1H- |
| | | indole-2-carboxylic acid |

Chemical structures of the compounds of Table 3 are as follows:

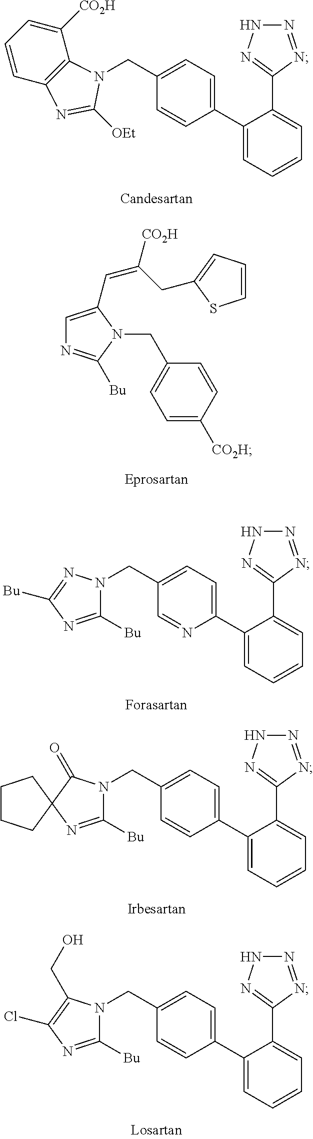

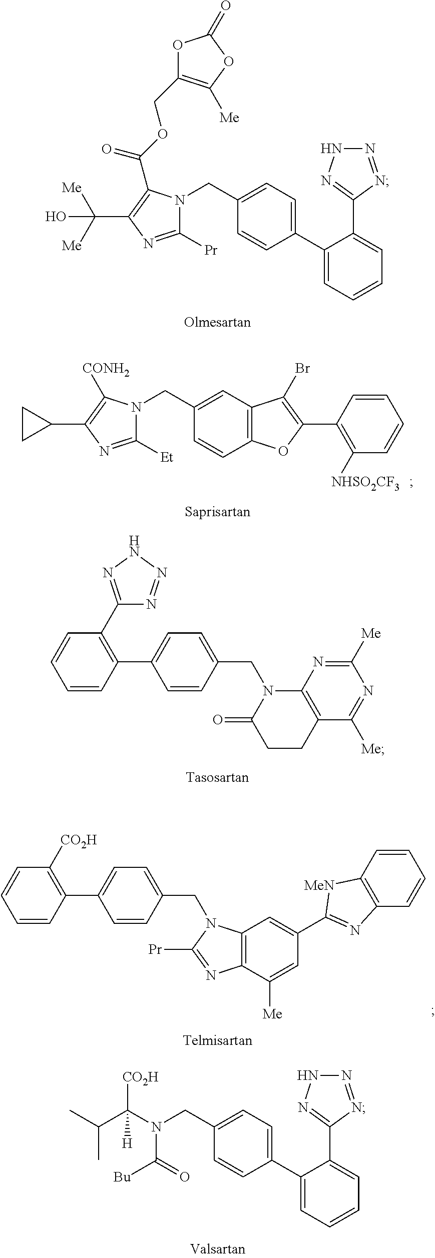

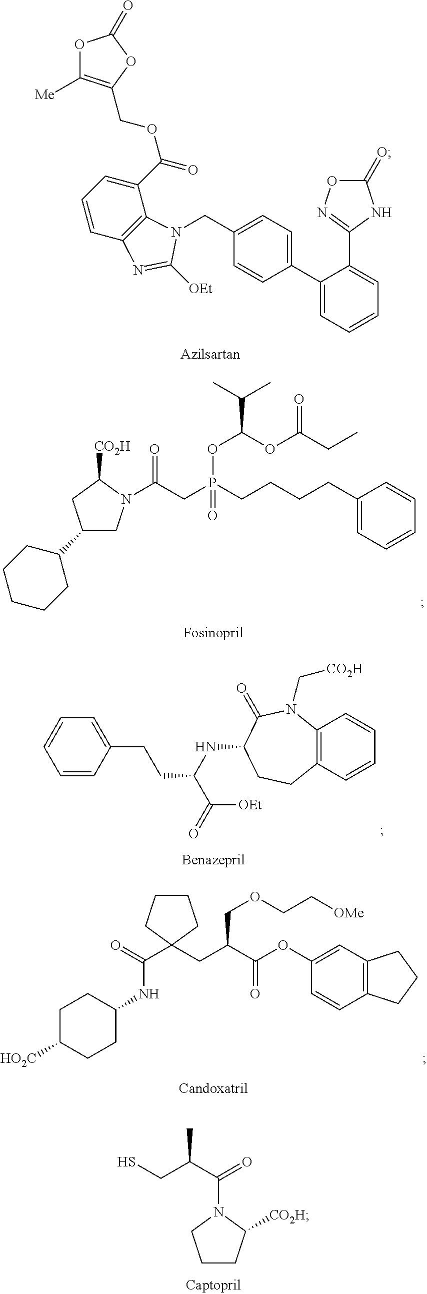 [SMILES: CCOc1nc2cccc(C(=O)OCc3oc(=O)oc3C)c2n1Cc1ccc(-c2ccccc2-c2noc(=O)[nH]2)cc1 ; CCC(=O)O[C@@H](OP(=O)(CCCCc1ccccc1)CC(=O)N1C[C@H](C2CCCCC2)C[C@H]1C(=O)O)C(C)C ; CCOC(=O)[C@H](CCc1ccccc1)N[C@H]1CCc2ccccc2N(CC(=O)O)C1=O ; COCCOC[C@H](CC1(C(=O)N[C@H]2CC[C@@H](C(=O)O)CC2)CCCC1)C(=O)Oc1ccc2c(c1)CCC2 ; C[C@H](CS)C(=O)N1CCC[C@H]1C(=O)O]

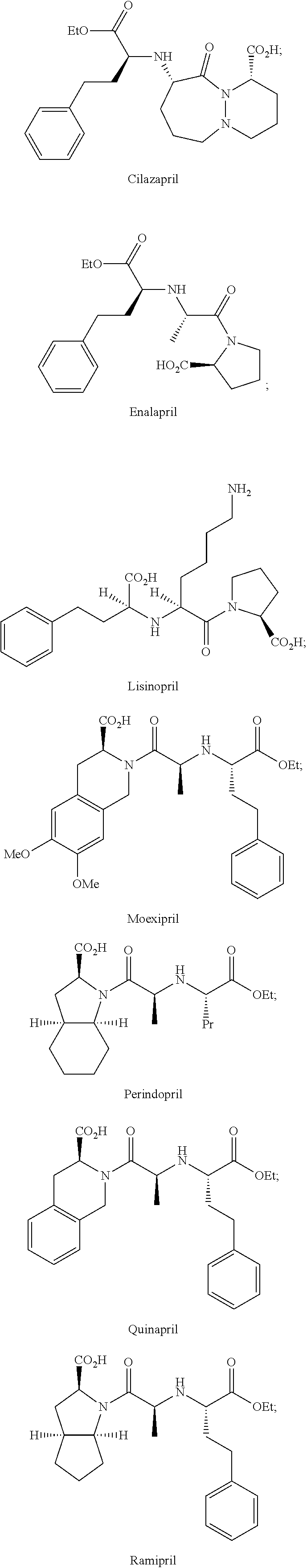

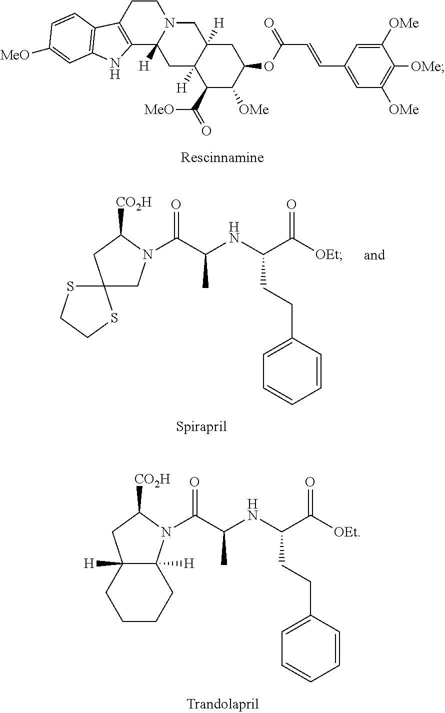 [SMILES: COC(=O)[C@H]1[C@H]2C[C@@H]3c4[nH]c5cc(OC)ccc5c4CCN3C[C@H]2C[C@@H](OC(=O)/C=C/c2cc(OC)c(OC)c(OC)c2)[C@@H]1OC ; CCOC(=O)[C@H](CCc1ccccc1)N[C@@H](C)C(=O)N1CC2(C[C@H]1C(=O)O)SCCS2 ; "and" ; CCOC(=O)[C@H](CCc1ccccc1)N[C@@H](C)C(=O)N1[C@H](C(=O)O)C[C@H]2CCCC[C@@H]21]

In some embodiments, a compound of the invention is a compound of the formula (III):

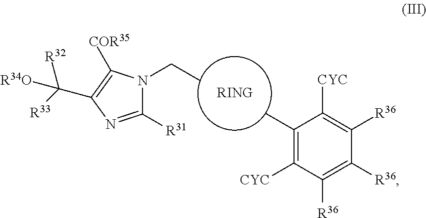

wherein:
R³¹ is H, halogen, OH, SH, cyano, nitro, alkyl, alkenyl, alkynyl, alkoxy, acyl, O-acyl, carbamato, a urethane, a carbonate, amino, mono-substituted amino, or di-substituted amino, wherein any of the foregoing is independently unsubstituted or substituted with any number of halogen atoms;
R³² is H, alkyl, alkenyl, alkynyl, alkoxy, aryl, aralkyloxy, or aryloxy;
R³³ is H, alkyl, alkenyl, alkynyl, alkoxy, aryl, aralkyloxy, or aryloxy;
R³⁴ is H, alkyl, alkenyl, alkynyl, acyl, carbamato, aryl, aralkyloxy, or aryloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
R³⁵ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
each R³⁶ is independently H, halogen, OH, SH, cyano, nitro, alkyl, alkenyl, alkynyl, alkoxy, acyl, O-acyl, carbamato, a urethane, a carbonate, amino, mono-substituted amino, or di-substituted amino, wherein any of the foregoing is independently unsubstituted or substituted with any number of halogen atoms;
each CYC is independently H, heterocycle, or heterocyclylalkyl; and
RING is a cyclic group having 4-8 members, wherein the RING group is saturated or unsaturated, and is unsubstituted or substituted with any number of alkyl groups and halogen atoms,
   or a pharmaceutically-acceptable salt thereof.

In some embodiments, R³¹ is H, halogen, OH, alkyl, alkoxy, acyl, O-acyl, amino, mono-substituted amino, or di-substituted amino, wherein any of the foregoing is independently unsubstituted or substituted with any number of halogen atoms; R³² is H, alkyl, alkoxy, aryl, or aryloxy; R³³ is H, alkyl, alkoxy, aryl, or aryloxy; R³⁴ is H, alkyl, acyl, or carbamato; R³⁵ is H, OH, alkyl, alkoxy, aryl, aralkyl, aryloxy, aralkyloxy, heterocyclyl, heterocyclylalkyl, O-heterocyclyl, or O-heterocyclylalkyl, wherein any of the foregoing is independently unsubstituted or substituted with any number of halogen atoms; each R³⁶ is independently H, halogen, OH, alkyl, alkoxy, acyl, O-acyl, amino, mono-substituted amino, or di-substituted amino, wherein any of the foregoing is independently unsubstituted or substituted with any number of halogen atoms; each CYC is independently H or a heterocycle; and RING is a cyclic group having 5 or 6 members, wherein the RING group is saturated or unsaturated, and is unsubstituted or substituted with any number of alkyl groups and halogen atoms, or a pharmaceutically-acceptable salt thereof.

In some embodiments, R³¹ is H, halogen, OH, alkyl, or alkoxy, wherein any of the foregoing is independently unsubstituted or substituted with any number of halogen atoms; R³² is H, alkyl, or aryl; R³³ is H, alkyl, or aryl; R³⁴ is H, alkyl, or acyl; R³⁵ is OH, alkoxy, aryloxy, aralkyloxy, O-heterocyclyl, O-heterocyclylalkyl, amino, mono-substituted amino, or di-substituted amino, wherein any of the foregoing is independently unsubstituted or substituted with any number of halogen atoms; each R³⁶ is independently H, halogen, OH, alkyl, or alkoxy; each CYC is independently H or a heterocycle containing at least one nitrogen atom; and RING is a 5-membered carbocycle or a 6-membered carbocycle, wherein the RING group is saturated or unsaturated, and is unsubstituted or substituted with any number of alkyl groups and halogen atoms, or a pharmaceutically-acceptable salt thereof.

In some embodiments, R³¹ is alkyl, which is unsubstituted or substituted with any number of halogen atoms; R³² is H or alkyl; R³³ is H or alkyl; R³⁴ is H, alkyl, or acyl; R³⁵ is O-heterocyclylalkyl; each R³⁶ is independently H, halogen, or OH; one CYC is H and the other CYC is a heterocycle containing at least one nitrogen atom; and RING is a 6-membered unsaturated carbocycle, wherein the RING group is saturated or unsaturated, and is unsubstituted or substituted with any number of alkyl groups and halogen atoms, or a pharmaceutically-acceptable salt thereof.

In some embodiments, R³¹ is alkyl; R³² is H or alkyl; R³³ is H or alkyl; R³⁴ is H or alkyl; R³⁵ is a group of formula:

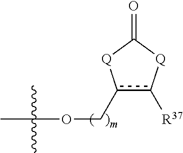

wherein each Q is independently O, S, or NH; R³⁷ is alkyl or aryl; m is 1, 2, 3, 4, or 5; and [symbol] is a single or a double bond;
   each R³⁶ is independently H, halogen, or OH; one CYC is H and the other CYC is a heterocycle containing at least one nitrogen atom; and RING is a 6-membered aromatic carbocycle, wherein the aromatic carbocycle is unsubstituted or substituted with any number of alkyl groups and halogen atoms, or a pharmaceutically-acceptable salt thereof.

In some embodiments, R³¹ is alkyl; R³² is H or alkyl; R³³ is H or alkyl; R³⁴ is H or alkyl; R³⁵ is a group of formula:

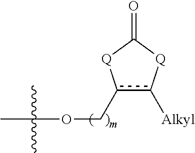

wherein each Q is independently O, S, or NH; m is 1, 2, 3, 4, or 5; and [symbol] is a single or a double bond;
   each R³⁶ is independently H, halogen, or OH; one CYC is H and the other CYC is a heterocycle containing at least one nitrogen atom; and RING is a 6-membered aromatic carbocycle, wherein the aromatic carbocycle is unsubstituted or substituted with any number of alkyl groups and halogen atoms, or a pharmaceutically-acceptable salt thereof.

In some embodiments, R³¹ is alkyl; R³² is H or alkyl; R³³ is H or alkyl; R³⁴ is H or alkyl; R³⁵ is (5-methyl-2-oxo-1,3-dioxolen-4-yl)methyloxy group, (5-ethyl-2-oxo-1,3-dioxolen-4-yl)methyloxy group, (5-phenyl-2-oxo-1,3-dioxolen-4-yl)methyloxy group; each R³⁶ is independently H, halogen, or OH; one CYC is H and the other CYC is a tetrazole; and RING is a 6-membered aromatic carbocycle, or a pharmaceutically-acceptable salt thereof.

In some embodiments, the compound is:

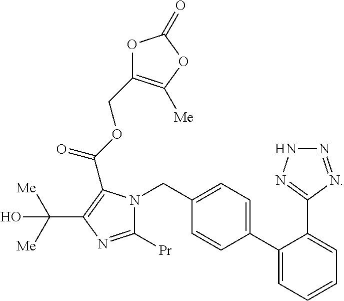

In some embodiments the compound is Olmesartan.

In some embodiments, a compound of the invention is a compound of the formula (IV):

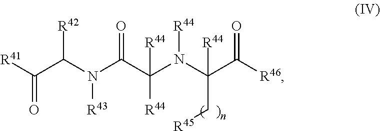

wherein:
R⁴¹ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
R⁴² and R⁴³ together with the atoms to which they are bonded form a ring;
each R⁴⁴ is independently H, alkyl, alkenyl, alkynyl, aryl, aralkyl, heterocyclyl, or heterocyclylalkyl, wherein any of the foregoing is independently unsubstituted or substituted with any number of halogen atoms;
R⁴⁵ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
R⁴⁶ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms; and
n is 1, 2, 3, 4, 5, 6, 7, 8, 9, or 10,
   or a pharmaceutically-acceptable salt thereof.

In some embodiments, a compound of the invention is a compound of the formula (V):

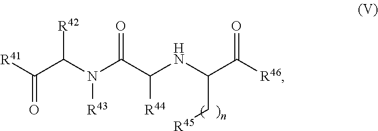

R⁴¹ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
R⁴² and R⁴³ together with the atoms to which they are bonded form a ring;
R⁴⁴ is H, alkyl, alkenyl, alkynyl, aryl, aralkyl, heterocyclyl, or heterocyclylalkyl, wherein any of the foregoing is independently unsubstituted or substituted with any number of halogen atoms;
R⁴⁵ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms;
R⁴⁶ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms; and
n is 1, 2, 3, 4, 5, 6, 7, 8, 9, or 10,
   or a pharmaceutically-acceptable salt thereof.

In some embodiments, R⁴¹ is OH, alkoxy, aryloxy, aralkyloxy, O-heterocyclyl, O-heterocyclylalkyl, amino, mono-substituted amino, or di-substituted amino, wherein any of the foregoing is independently unsubstituted or substituted with any number of halogen atoms; R⁴² and R⁴³ together with the atoms to which they are bonded form a monocyclic or polycyclic heterocycle; R⁴⁴ is H, alkyl, alkenyl, alkynyl, aryl, aralkyl, heterocyclyl, or heterocyclylalkyl, wherein any of the foregoing is independently unsubstituted or substituted with any number of halogen atoms; R⁴⁵ is H, OH, SH, alkyl, alkenyl, alkynyl, alkoxy, aryl, aralkyl, aryloxy, aralkyloxy, heterocyclyl, heterocyclylalkyl, O-heterocyclyl, O-heterocyclylalkyl, amino, mono-substituted amino, or di-substituted amino, wherein any of the foregoing is independently unsubstituted or substituted with any number of halogen atoms; R⁴⁶ is OH, alkoxy, aryloxy, aralkyloxy, O-heterocyclyl, O-heterocyclylalkyl, amino, mono-substituted amino, or di-substituted amino, wherein any of the foregoing is independently unsubstituted or substituted with any number of halogen atoms; and n is 1, 2, 3, 4, 5, 6, 7, 8, 9, or 10, or a pharmaceutically-acceptable salt thereof.

In some embodiments:
R⁴¹ is OH, alkoxy, aryloxy, aralkyloxy, O-heterocyclyl, or O-heterocyclylalkyl, wherein any of the foregoing is independently unsubstituted or substituted with any number of halogen atoms;
R⁴² and R⁴³ together with the atoms to which they are bonded form a monocyclic or polycyclic heterocycle, wherein the monocyclic or polycyclic heterocycle has from 5 to 15 ring atoms;
R⁴⁴ is H, alkyl, alkenyl, or alkynyl, wherein any of the foregoing is independently unsubstituted or substituted with any number of halogen atoms;
R⁴⁵ is H, alkyl, alkenyl, alkynyl, aryl, aralkyl, heterocyclyl, or heterocyclylalkyl, wherein any of the foregoing is independently unsubstituted or substituted with any number of halogen atoms;
R⁴⁶ is OH, alkoxy, aryloxy, aralkyloxy, O-heterocyclyl, or O-heterocyclylalkyl, wherein any of the foregoing is independently unsubstituted or substituted with any number of halogen atoms; and
n is 1, 2, 3, 4, or 5,
   or a pharmaceutically-acceptable salt thereof.

In some embodiments, R⁴¹ is OH or alkoxy; R⁴² and R⁴³ together with the atoms to which they are bonded form a substituted or unsubstituted tetrahydroisoquinoline, a substituted or unsubstituted tetrahydroquinoline, a substituted or unsubstituted octahydroindole, or a substituted or unsubstituted octahydrocyclopenta[b]pyrrole; R⁴⁴ is H or alkyl; R⁴⁵ is H, alkyl, or aryl; R⁴⁶ is OH or alkoxy; and n is 1, 2, or 3, or a pharmaceutically-acceptable salt thereof.

In some embodiments, the compound is:

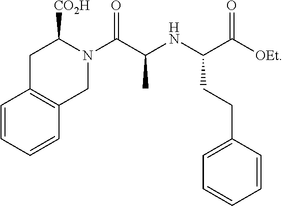 [SMILES: CCOC(=O)[C@H](CCc1ccccc1)N[C@@H](C)C(=O)N1Cc2ccccc2C[C@H]1C(=O)O]

In some embodiments the compound is Quinapril.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, and ester groups.

Non-limiting examples of alkyl groups include straight, branched, and cyclic alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl groups include straight, branched, and cyclic alkenyl groups. The olefin or olefins of an alkenyl group can be, for example, E, Z, cis, trans, terminal, or exo-methylene.

Non-limiting examples of alkynyl groups include straight, branched, and cyclic alkynyl groups. The triple bond of an alkylnyl group can be internal or terminal.

A halo group can be any halogen atom, for example, fluorine, chlorine, bromine, or iodine.

A halo-alkyl group can be any alkyl group substituted with any number of halogen atoms, for example, fluorine, chlorine, bromine, and iodine atoms. A halo alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon. A heterocycle can be substituted with any number of substituents, for example, alkyl groups and halogen atoms. A heterocycle can be aromatic or non aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

The invention provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, a iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by any form and route known in the art including, for example, intravenous, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically acceptable carriers or excipients well known in the art. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which may optionally contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parental injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved and/or dispersed in a polymer or an adhesive. Such patches may be constructed for continuous, pulsatile, or on demand delivery of pharmaceutical compounds. Transdermal delivery can be accomplished by means of iontophoretic patches and the like. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and means to secure the device to the skin.

For administration by inhalation, the active compounds can be in a form as an aerosol, a mist, or a powder. Pharmaceutical compositions are conveniently delivered in the form of an aerosol spray presentation from pressurized packs or a nebuliser, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, is first melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compounds described herein can be manufactured in a conventional manner, for example, by means of conventional mixing, dissolving, granulating, dragee-making, levigating, emulsifying, encapsulating, entrapping or compression processes.

The pharmaceutical compositions can include at least one pharmaceutically acceptable carrier, diluent, or excipient and compounds described herein as free-base or pharmaceutically-acceptable salt form. The methods and pharmaceutical compositions described herein include the use crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to prevent premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the invention include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the invention include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, granular masses, and the like.

Compositions of the invention can be delivered via a time-controlled delivery system. An example of a suitable time-controlled delivery system is the PULSINCAP® system, or a variant thereof. The time-controlled delivery system can further comprise pH-dependent systems, microbially-triggered delivery systems, or a combination thereof. The time-controlled system may comprise a water insoluble capsule body enclosing a drug reservoir. The capsule body can be closed at one end with a hydrogel plug. The hydrogel plug can comprise swellable polymers, erodible compressed polymers, congealed melted polymers, enzymatically-controlled erodible polymers, or a combination thereof. The swellable polymers can include polymethacrylates. Non-limiting examples of erodible compressed polymers include hydroxypropyl methylcellulose, polyvinyl alcohol, polyvinyl acetate, polyethylene oxide, and combinations thereof. Non-limiting examples of congealed melted polymers include saturated polyglycolated glycerides, glyceryl monooleate, and combinations thereof. Non-limiting examples of enzymatically-controlled erodible polymers include polysaccharides; amylose; guar gum; pectin; chitosan; inulin; cyclodextrin; chondroitin sulphate; dextrans; locust bean gum; arabinogalactan; chondroitin sulfate; xylan; calcium pectinate; pectin/chitosan mixtures; amidated pectin; and combinations thereof.

The time-controlled delivery system can comprise a capsule, which further comprises an organic acid. The organic acid can be filled into the body of a hard gelatine capsule. The capsule can be coated with multiple layers of polymers. The capsule can be coated first with an acid soluble polymer, such as EUDRAGIT® E, then with a hydrophilic polymer, such as hydroxypropyl methylcellulose, and finally with an enteric coating, such as EUDRAGIT® L.

An additional example of a suitable time-controlled delivery system is the CHRONOTROPIC® system, or a variant thereof, which comprises a drug core that is coated with hydroxypropyl methylcellulose and an outer enteric film.

An additional example of a suitable time-controlled delivery system is the CODES™ system, or a variant thereof. The time-controlled delivery system can comprise a capsule body, which can house, for example, a drug-containing tablet, an erodible tablet, a swelling expulsion excipient, or any combination thereof. The capsule can comprise an ethyl cellulose coat. The time-controlled delivery system can comprise two different sized capsules, one inside the other. The space between the capsules can comprise a hydrophilic polymer. The drug-containing core canay be housed within the inner capsule. The drug delivery system can comprise an impermeable shell, a drug-containing core, and erodible outer layers at each open end. When the outer layers erode, the drug is released.

Examples of suitable multiparticulate drug delivery systems include DIFFUCAPS®, DIFFUTAB®, ORBEXA®, EURAND MINITABS®, MICROCAPS®, and variants thereof. The drug delivery system can comprise multiparticulate beads, which are comprised of multiple layers of the drug compound, excipients, and release-controlling polymers. The multiparticulate beads can comprise an organic acid or alkaline buffer. The multiparticulate beads can comprise a solid solution of the drug compound and crystallization inhibitor. The drug delivery system can comprise a matrix tablet containing water-soluble particles and the drug compound. The matrix tablet can further comprise hydrophilic and hydrophobic polymers. In some multiparticulate delivery systems, particles in the micron size range are used. In some multiparticulate delivery systems, nanoparticle colloidal carriers composed of natural or synthetic polymers are used.

In some embodiments, a controlled release formulation is a delayed release form. A delayed release form can be formulated to delay a compound's action for an extended period of time. A delayed release form can be formulated to delay the release of an effective dose of one or more compounds, for example, for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, or about 44 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, or about 44 hours.

A tablet providing a sustained or controlled release can comprise a first layer containing one or two of the compounds described herein, and a tablet core containing one or two other compounds. The core can have a delayed or sustained dissolution rate. Other exemplary embodiments can include a barrier between the first layer and core, to limit drug release from the surface of the core. Barriers can prevent dissolution of the core when the pharmaceutical formulation is first exposed to gastric fluid. For example, a barrier can comprise a disintegrant, a dissolution-retarding coating (e.g., a polymeric material, for example, an enteric polymer such as a Eudragit polymer), or a hydrophobic coating or film, and can be selectively soluble in either the stomach or intestinal fluids. Such barriers permit the compounds to leach out slowly. The barriers can cover substantially the whole surface of the core.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 5 mg, about 10 mg, about 20 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In some embodiments, a dose can be expressed in terms of an amount of the drug divided by the mass of the subject, for example, milligrams of drug per kilograms of subject body mass. In some embodiments, CW299 is present in a composition in an amount ranging from about 5 mg/kg to about 1600 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, CW304 is present in a composition in an amount ranging from about 0.001 mg/kg to about 30 mg/kg, about 0.01 mg/kg to about 15 mg/kg, about 0.1 mg/kg to about 5 mg/kg or about 1 mg/kg to about 2 mg/kg. In some embodiments, CW330 is present in a composition in an amount ranging from about 1 mg/kg to about 800 mg/kg, about 4 mg/kg to about 200 mg/kg, about 8 mg/kg to about 50 mg/kg or about 10 mg/kg to about 25 mg/kg.

In some embodiments, a composition comprises from about 10 mg to about 800 mg of CW299, from about 0.1 mg to about 2 mg of CW304, and from about 1 mg to about 50 mg CW330.

In some embodiments, a compound described herein is present in a composition in an amount that is a fraction or percentage of the maximum tolerated amount.

The maximum tolerated amount can be as determined in a subject, such as a mouse or human.

The fraction can be expressed as a ratio of the amount present in the composition divided by the maximum tolerated dose. The ratio can be from about 1/20 to about 1/1. The ratio can be about 1/20, about 1/19, about 1/18, about 1/17, about 1/16, about 1/15, about 1/14, about 1/13, about 1/12, about 1/11, about 1/10, about 1/9, about 1/8, about 1/7, about 1/6, about 1/5, about 1/4, about 1/3, about 1/2, or about 1/1. The ratio can be 1/20, 1/19, 1/18, 1/17, 1/16, 1/15, 1/14, 1/13, 1/12, 1/11, 1/10, 1/9, 1/8, 1/7, 1/6, 1/5, 1/4, 1/3, 1/2, or 1/1. The ratio can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The ratio can be in a range from about 5% to about 100%, from about 10% to about 100%, from about 5% to about 80%, from about 10% to about 80%, from about 5% to about 60%, from about 10% to about 60%, from about 5% to about 50%, from about 10% to about 50%, from about 5% to about 40%, from about 10% to about 40%, from about 5% to about 20%, or from about 10% to about 20%.

For example, the maximum tolerated dose of imatinib mesylate is 100 mg/kg in mice. The maximum tolerated dose of roflumilast is 2 mg/kg in mice. The maximum tolerated dose of olmesartan or quinapril is 25 mg/kg in mice.

The foregoing ranges are merely suggestive. Dosages can be altered depending on a number of variables, including, for example, the activity of the compound used, the disease or condition to be treated, the mode of administration, the requirements of the individual subject, the severity of the disease or condition being treated, and the judgment of the practitioner.

A dose can be modulated to achieve a desired pharmacokinetic or pharmacodynamics profile, such as a desired or effective blood profile, as described herein.

### Pharmacokinetic and Pharmacodynamic Measurements

Pharmacokinetic and pharmacodynamic data can be obtained by techniques known in the art. Appropriate pharmacokinetic and pharmacodynamic profile components describing a particular composition can vary due to the inherent variation in pharmacokinetic and pharmacodynamic parameters of drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 16 subjects, 20 subjects, 25 subjects, 30 subjects, 35 subjects, or more. The mean is determined by calculating the average of all subject's measurements for each parameter measured.

The pharmacokinetic parameters can be any parameters suitable for describing a compound disclosed herein. For example, the Cₘₐₓ can be not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of a compound described herein.

The Tₘₐₓ of a compound described herein can be, for example, not greater than about 0.5 hours, not greater than about 1.0 hours, not greater than about 1.5 hours, not greater than about 2.0 hours, not greater than about 2.5 hours, not greater than about 3.0 hours, or any other Tₘₐₓ appropriate for describing a pharmacokinetic profile of a compound described herein.

The AUC_{(0-inf)} of a compound described herein can be, for example, not less than about 250 ng·hr/mL, not less than about 500 ng·hr/mL, not less than about 1000 ng·hr/mL, not less than about 1500 ng·hr/mL, not less than about 2000 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, or any other AUC_{(0-inf)} appropriate for describing a pharmacokinetic profile of a compound described herein.

The plasma concentration of a compound described herein about one hour after administration can be, for example, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a compound described herein.

The pharmacodynamic parameters can be any parameters suitable for describing compositions of the invention. For example, the pharmacodynamic profile can exhibit decreases in factors associated with inflammation after, for example, about 2 hours, about 4 hours, about 8 hours, about 12 hours, or about 24 hours.

For example, for Imatinib, the Tₘₐₓ is 1.2 hours; the T½ is 13 hours (with a peak at 17.5 hours for an active metabolite), and the Cₘₐₓ is 0.925 μg/mL (with a peak at 0.115 μL for an active metabolite). For Roflumilast, the Tₘₐₓ is 1 hour; the T½ is 12-14 hours, and the Cₘₐₓ is 7.04 μg/mL. For Olmesartan or Quinapril, the Tₘₐₓ is 1-2 hours; the T½ is 13 hours, and the Cₘₐₓ is 0.22-2.1 μg/mL.

### Physicochemical Properties & Mechanism of Action

In some embodiments, an inhibitor of any of colony stimulating factor, platelet derived growth factor, T-cell response and a B-cell response pathway can be the compound Imatinib. Pharmaceutical compositions formulated with Imanitib can be called, for example, Imatinib Mesylate, Imatinib Methansulfonate, STI-571. Imatininb can act, for example, as a kinase inhibitor, a protein kinase inhibitor, or an antineoplastic agent. In some embodiments, imatinib or a salt thereof is crystalline. Imatinib mesylate can be soluble in water at pH<5.5, and slightly soluble to insoluble in neutral/alkaline aqueous buffers. Imatininb mesylate can be freely soluble to very slightly soluble in dimethyl sulfoxide, methanol and ethanol, and can be insoluble in n-octanol, acetone, and acetonitrile.

Imatininb or a salt thereof can be well-absorbed with mean absolute bioavailability is 98%, with maximum levels achieved within 2-4 hours of dosing. Imatininb protein binding can be about 95%. Imatinib can suffer metabolism, for example, by CYP3A4 CYP1A2, CYP2D6, CYP2C9, and CYP2C19. In some embodiments, metabolisms provides an active metabolite in the form of an N-demethylated piperazine derivative.

Imatininb can have a half-life of about 18 hours. An active metabolite of imatininb can have a half-life of about 40 hours.

Side effects of imaitninb therapy include, for example, vomiting, diarrhea, loss of appetite, dry skin, hair loss, swelling (especially in the legs or around the eyes) and muscle cramps.

In some embodiments, at pharmaceutically obtainable concentrations (for example, Cmax=4.6 uM at 400 mg/day), Imatinib inhibits the tyrosine kinases Abl (IC50=0.22 uM), the abl-related gene product (IC50=0.5 uM), collagen-induced discoiddin domain receptor 1 (IC50═0.34 uM) and DDR2 (IC50=0.68 uM), the PDGFR family members stem cell factor receptor (c-kit; 0.1 uM), PDGFR-alpha and -beta (IC50=0.1 uM) and the macrophage colony stimulating factor (M-CSF) receptor, c-fms (IC50=1.4 uM).

In some embodiments, imatinib inhibits nontyrosine kinase targets NAD(P)H: quinone oxidoreductase 2 (IC50=0.08 uM) and some members of the carbonic anhydrase (CA) family of metalloproteases, including human CA (hCA) II (IC50=0.03 uM) and hCA XIV (IC50=0.47 uM).

In some embodiments, an inhibitor of phosphodiesterase 4 can be the compound roflumilast. Roflumilast can act, for example, as a bronchodilator or a phosphodiesterase-4 inhibitor. In some embodiments, roflumilast is a white to off-white non-hygroscopic powder. Roflumilast can be sparingly soluble in ethanol and freely soluble in acetone.

Roflumilast is a potent and highly selective inhibitor of the enzyme PDE4. PDE4 is expressed in inflammatory cells such as neutrophils and macrophages. PDE4 degrades the second messenger cyclic AMP (cAMP). High levels of intracellular cAMP reduce the activity of inflammatory cells; therefore roflumilast increases intracellular cAMP levels and reduces inflammatory cell activity.

Roflumilast can have rapid absorption, with an absolute bioavailability of, for example, 79%. Roflumilast protein binding can be about 96%, or greater than about 96%. can be about can suffer metabolism, for example, by cytochrome-P 450 (CYP) 3A4 and CYP1A2 to an active metabolite, for example, roflumilast N-oxide.

Roflumilast can have a half life of about 14 to about 16 hours. Roflumilast N-oxide can have a half life of about 19 to about 21 hours.

Side effects of roflumilast therapy include, for example, gastrointestinal (GI) events including nausea, diarrhea, and weight loss.

In some embodiments of the present invention, an inhibitor of Angiotensin II Type 1 Receptor is Olmesartan. Pharmaceutical compositions formulated with Olmesartan can be called, for example, DE-092 and Olmesartan medoximil. Olmesartan can act, for example, as an antihypertensive agent, or an angiotensin II type 1 receptor blocker (ARB). In some embodiments, Olmesartan can be a white to light yellowish-white powder or crystalline powder. Olmesartan can be practically insoluble in water and sparingly soluble in methanol.

Olmesartan medoxomil can be rapidly bioactivated by ester hydrolysis to olmesartan during absorption from the gastrointestinal tract. The absolute bioavailability can be about 26%.

Olmesartan protein binding can be 99%. In some embodiments, olmesartan does not penetrate red blood cells.

In some embodiments, olmesartan is not metabolized. The olmesartan half-life can be about 13 hours.

Side effects of olmesartan therapy include, for example, dehydration (dry mouth, excessive thirst, muscle pain or cramps, nausea and vomiting, weakness), dizziness, low blood pressure, and slow or irregular heartbeat.

In some embodiments, olmesartan blocks the vasoconstrictor effects of angiotensin II by selectively blocking the binding of angiotensin II to the AT1 receptor. Angiotensin II is formed from angiotensin I in a reaction catalyzed by angiotensin converting enzyme (ACE). Angiotensin II is the principal pressor agent of the renin-angiotensin system. Olmesartan has more than a 12,500-fold greater affinity for the AT1 receptor than for the AT2 receptor.

In some embodiments, the inhibitor of Angiotensin-converting Enzyme is quinapril. Pharmaceutical compositions formulated with quinapril can be called, for example, Quinapril HCl, Quinapril Hydrochloride and Quinaprilum. Quinapril can act, for example, as an antihypertensive agent, or an angiotensin-converting enzyme inhibitor.

In some embodiments, quinapril is a white to off-white amorphous powder. Quinapril can be freely soluble in aqueous solvents.

Quinapril can have a peak plasma concentration, for example, within one hour following oral administration. The extent of absorption can be, for example, at least 60%. Quinapril protein binding can be, for example, about 97%. Quinapril metabolism can be, for example, hepatic. Quinapril half-life can be, for example, 2 hours with a prolonged terminal phase of 25 hours.

Side effects of imaitninb therapy include, for example, hypotension, dizziness, cough, chest pain, dyspnea, fatigue, and nausea/vomiting.

In some embodiments, Quinapril is deesterified to the principal metabolite, quinaprilat, Quinaprilat, an active metabolite of quinapril, can compete with ATI for binding to ACE and can inhibit enzymatic proteolysis of ATI to ATII. Decreasing ATII levels in the body can decrease blood pressure by inhibiting the pressor effects of ATII.

### Inflammatory Conditions and Methods of Treatment

In some embodiments, the invention provides a process for preparing a composition, the composition comprising one or more compounds, wherein the compounds are CW299, CW304 and CW330, wherein the composition optionally further comprises a pharmaceutically-acceptable excipient, wherein the process comprises the step of combining the compounds and the optional excipient in any order thereof.

The invention described herein provides therapeutic methods for the treatment of inflammatory joint diseases and chronic inflammatory connective tissue diseases, or combinations thereof.

In one embodiment of the present disclosure, the inflammatory joint diseases is selected from a group comprising but not limiting to rheumatoid arthritis, juvenile RA (JRA), osteoarthritis, polyarthritis, spondylitis, bursitis and gout or any combination of diseases thereof. The allied arthritic diseases also include psoriatic arthritis, ankylosing spondylitis, infectious arthritis including reactive arthritis; intestinal diseases including ulcerative colitis, inflammatory bowel disease (IBD) and the like or any combination of allied diseases thereof.

In one embodiment of the present disclosure, the chronic inflammatory connective tissue diseases are selected from a group comprising but not limiting to systemic lupus erythematosus, scleroderma, Sjorgen's syndrome, poly- and dermatomyositis, vasculitis, mixed connective tissue disease (MCTD), tendonitis, synovitis, bacterial endocarditis, osteomyelitis and psoriasis or any combination of diseases thereof.

In some embodiments, the invention provides a method for treating inflammatory joint diseases and/or chronic inflammatory connective tissue diseases in a subject in need or want of relief thereof, the method comprising administering to the subject: a) a therapeutically-effective amount of an inhibitor of one of colony stimulating factor, platelet derived growth factor, T-cell response and B-cell response pathways; b) a therapeutically-effective amount of an inhibitor of phosphodiesterase 4; and c) a therapeutically-effective amount of an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a medicament for the treatment of inflammatory joint diseases and/or chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a medicament for the treatment of inflammatory joint diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a medicament for the treatment of chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor of angiotensin II AT1 receptors or an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a kit for the treatment of inflammatory joint diseases and/or chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a kit for the treatment of inflammatory joint diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a use of a combination of compounds in the preparation of a kit for the treatment of chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a combination of compounds for use in the treatment of inflammatory joint diseases and/or chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a combination of compounds for use in the treatment of inflammatory joint diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

In some embodiments the invention provides a combination of compounds for use in the treatment of chronic inflammatory connective tissue diseases, the compounds comprising: an inhibitor of one of colony stimulating factor, platelet derived growth factor, T-cell response and B-cell response pathways; an inhibitor of phosphodiesterase 4; and an inhibitor associated with angiotensin. An inhibitor associated with angiotensin can be an inhibitor of angiotensin II AT1 receptors or an inhibitor of angiotensin-converting enzyme.

Pharmaceutical compositions containing compounds described herein can be administered for prophylactic and/or therapeutic treatments. In therapeutic applications, the compositions are administered to a subject already suffering from a disease or condition, in an amount sufficient to cure or at least partially arrest the symptoms of the disease or condition, or to cure, heal, improve, or ameliorate the condition itself. Amounts effective for this use can vary based on the severity and course of the disease or condition, previous therapy, the subject's health status, weight, and response to the drugs, and the judgment of the treating physician. Pharmaceutically-acceptable amounts can be determined by routine experimentation, for example, by a dose escalation clinical trial.

Multiple therapeutic agents can be administered in any order or simultaneously. If simultaneously, the multiple therapeutic agents can be provided in a single, unified form, or in multiple forms, for example, as multiple separate pills. The compounds can be packed together or separately, in a single package or in a plurality of packages. One or all of the therapeutic agents can be given in multiple doses. If not simultaneous, the timing between the multiple doses may vary to as much as about a month.

In some embodiments, compounds of the invention are administered sequentially at a time interval. The time interval can range from about 1 second to about 600 minutes.

Compounds and compositions of the invention can be packaged as a kit. In some embodiments, a kit includes written instructions on the use of the compounds and compositions. The instructions can provide information on the identity of the therapeutic agent(s), modes of administration, or the indications for which the therapeutic agent(s) can be used.

In some embodiments, therapeutics are combined with genetic or genomic testing to determine whether that individual is a carrier of a mutant gene that is known to be correlated with certain diseases or conditions. A personalized medicine approach can be used to provide companion diagnostic tests to discover a subject's predisposition to certain conditions and susceptibility to therapy. For example, a subject who is an anti-TNF non-responder could be identified via companion diagnostics. The companion diagnostic test can be performed on a tissue sample of the subject, such as blood, hair, or skin.

Instructions on the use of a companion diagnostic test can be provided on written material packaged with a compound, composition, or kit of the invention. The written material can be, for example, a label. The written material can suggest conditions or genetic features relevant to inflammation or the therapeutic compounds of the invention. The instructions provide the subject and the supervising physician with the best guidance for achieving the optimal clinical outcome from the administration of the therapy.

Compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms.

The initial administration can be via any route practical, such as by any route described herein using any formulation described herein. A compound can be administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria.

In some embodiments, provided herein is a method of treating juvenile idiopathic arthritis using a therapeutically-effective amount of a composition provided herein. Juvenile idiopathic arthritis (JIA) (aka Juvenile Rheumatoid Arthritis JRA) is an autoimmune disorder. Onset is typically before age 16. The cardinal clinical feature is persistent swelling of the affected joint(s), which commonly include the knee, ankle, wrist and small joints of the hands and feet. Other joints affected can include spine, sacroiliac joints, shoulder, hip and jaw. JIA may be transient and self limited or chronic, and differs significantly from arthritis commonly seen in adults.

In some embodiments, provided herein is a method of treating psoriatic arthritis using a therapeutically-effective amount of a composition provided herein. Psoriatic arthritis is a type of inflammatory arthritis. People who have the chronic skin condition psoriasis are likely to develop this type of arthritis. Psoriatic arthritis is said to be a seronegative spondyloarthropathy and therefore occurs more commonly in patients with tissue type HLA-B27.

In some embodiments, provided herein is a method of treating plaque psoriasis using a therapeutically-effective amount of a composition provided herein. Plaque psoriasis is an autoimmune disease that appears on the skin. It is one of the five known types of psoriasis. In psoriasis, immune cells move from the dermis to the epidermis, where they stimulate skin cells (keratinocytes) to proliferate. Immune cells, such as dendritic cells and T cells, move from the dermis to the epidermis, secreting chemical signals, such as tumor necrosis factor-α, interleukin-1β, and interleukin-6, which cause inflammation, and interleukin-22, which causes keratinocytes to proliferate. In plaque psoriasis, skin rapidly accumulates at elbows and knees, but can affect any area, including the scalp, palms of hands and soles of feet, and genitals which gives it a silvery-white appearance.

In some embodiments, provided herein is a method of treating osteoarthritis using a therapeutically-effective amount of a composition provided herein. Osteoarthritis also known as degenerative arthritis or degenerative joint disease or osteoarthrosis, is a group of mechanical abnormalities involving degradation of joints, including articular cartilage and subchondral bone. Symptoms may include joint pain, tenderness, stiffness, locking, and sometimes an effusion. When bone surfaces become less well protected by cartilage, bone may be exposed and damaged. As a result of decreased movement secondary to pain, regional muscles may atrophy, and ligaments may become more lax.

In some embodiments, provided herein is a method of treating polyarthritis using a therapeutically-effective amount of a composition provided herein. Polyarthritis is any type of arthritis which involves five or more joints simultaneously. It is usually associated with autoimmune conditions. Polyarthritis is most often caused by an auto-immune disorder such as Rheumatoid arthritis, Psoriatic arthritis, and Lupus erythematosus but can also be caused by infection with an alphavirus such as Chikungunya Virus and Ross River Virus.

In some embodiments, provided herein is a method of treating spondylitis using a therapeutically-effective amount of a composition provided herein. Spondylitis is an inflammation of the vertebra. It is a form of spondylopathy. In many cases, spondylitis involves one or more vertebral joint as well, which itself is called spondylarthritis.

In some embodiments, provided herein is a method of treating bursitis using a therapeutically-effective amount of a composition provided herein. Bursitis is the inflammation of one or more bursae (small sacs) of synovial fluid in the body. The bursae rest at the points where internal functionaries, such as muscles and tendons, slide across bone.

Healthy bursae create a smooth, almost frictionless functional gliding surface making normal movement painless. When bursitis occurs, however, movement relying upon the inflamed bursa becomes difficult and painful. Moreover, movement of tendons and muscles over the inflamed bursa aggravates its inflammation, perpetuating the problem.

In some embodiments, provided herein is a method of treating gout using a therapeutically-effective amount of a composition provided herein. Gout is characterized by recurrent attacks of acute inflammatory arthritis—a red, tender, hot, swollen joint. Gout is caused by elevated levels of uric acid in the blood which crystallizes and the crystals are deposited in joints, tendons, and surrounding tissues. The metatarsal-phalangeal joint at the base of the big toe is commonly affected. However, gout may also present as tophi, kidney stones, or urate nephropathy.

In some embodiments, provided herein is a method of treating arthritic diseases using a therapeutically-effective amount of a composition provided herein.

In some embodiments, provided herein is a method of treating ankylosing spondylitis (AS) using a therapeutically-effective amount of a composition provided herein. Ankylosing spondylitis (previously known as Bekhterev's disease) is a chronic inflammatory disease of the axial skeleton with variable involvement of peripheral joints and nonarticular structures. AS is a form of spondyloarthritis, a chronic, inflammatory arthritis and autoimmune disease. It mainly affects joints in the spine and the sacroiliac joint in the pelvis, and can cause eventual fusion of the spine results in a complete rigidity of the spine, a condition known as bamboo spine. Both tumor necrosis factor-alpha (TNF α) and IL-1 are also implicated in ankylosing spondylitis.

In some embodiments, provided herein is a method of treating infectious arthritis including but not limited to osteomyelitis using a therapeutically effective amount of a composition provided herein. Staphylococcus aureus is the most common organism seen in osteomyelitis, seeded from areas of contiguous infection. But anaerobes and Gram-negative organisms, including Pseudomonas aeruginosa, E. coli, and Serratia marcescens, are also common. Mixed infections are the rule rather than the exception. Septic arthritis is the purulent invasion of a joint by an infectious agent which produces arthritis. Systemic mycotic (fungal) infections may also cause osteomyelitis. The two most common are Blastomyces dermatitidis and Coccidioides immitis.

In some embodiments, provided herein is a method of treating reactive arthritis using a therapeutically-effective amount of a composition provided herein. Reactive arthritis is classified as an autoimmune condition that develops in response to an infection in another part of the body (cross-reactivity). Coming into contact with bacteria and developing an infection can trigger the disease. It is set off by a preceding infection, the most common of which would be a genital infection with Chlamydia trachomatis. Other bacteria known to cause reactive arthritis which are more common worldwide are Ureaplasma urealyticum, Salmonella spp., Shigella spp., Yersinia spp., and Campylobacter spp. Synovial fluid cultures are negative, suggesting that reactive arthritis is caused either by an over-stimulated autoimmune response or by bacterial antigens which have somehow become deposited in the joints.

In some embodiments, provided herein is a method of treating intestinal diseases including ulcerative colitis, inflammatory bowel disease (IBD) using a therapeutically-effective amount of a composition provided herein. IBD involves chronic inflammation of all or part of your digestive tract. IBD is a group of inflammatory conditions of the colon and small intestine. The major types of IBD are Crohn's disease and ulcerative colitis.

In some embodiments, provided herein is a method of treating Systemic lupus erythematosus (SLE) using a therapeutically-effective amount of a composition provided herein. In SLE, the body's immune system produces antibodies against itself, particularly against proteins in the cell nucleus. SLE is triggered by environmental factors that are unknown. During an immune reaction to a foreign stimulus, such as bacteria, virus, or allergen, immune cells that would normally be deactivated due to their affinity for self tissues can be abnormally activated by signaling sequences of antigen-presenting cells. Thus triggers may include viruses, bacteria, allergens (both IgE and hypersensitivity), and can be aggravated by environmental stimulants such as ultraviolet light and certain drug reactions. These stimuli begin a reaction that leads to destruction of other cells in the body and exposure of their DNA, histones, and other proteins, particularly parts of the cell nucleus. The body's sensitized B-lymphocyte cells will now produce antibodies against these nuclear-related proteins. These antibodies clump into antibody-protein complexes which stick to surfaces and damage blood vessels in critical areas of the body, such as the glomeruli of the kidney; these antibody attacks are the cause of SLE. SLE is a chronic inflammatory disease believed to be a type III hypersensitivity response with potential type II involvement. HMGB1 may contribute to the pathogenesis of chronic inflammatory and autoimmune diseases due to its proinflammatory and immunostimulatory properties.

In some embodiments, provided herein is a method of treating Sjögren's syndrome using a therapeutically-effective amount of a composition provided herein. Sjögren's syndrome is a systemic autoimmune disease in which immune cells attack and destroy the exocrine glands that produce tears and saliva. The hallmark symptom of Sjögren's syndrome is a generalized dryness, typically including xerostomia (dry mouth) and xerophthalmia (dry eyes), part of what are known as sicca symptoms. In addition, Sjögren's syndrome may cause skin, nose, and vaginal dryness, and may affect other organs of the body, including the kidneys, blood vessels, lungs, liver, pancreas, peripheral nervous system (distal axonal sensorimotor neuropathy) and brain. Sjögren's syndrome is associated with increased levels in Cerebrospinal fluid (CSF) of IL-1RA, an interleukin 1 antagonist. This suggests that the disease begins with increased activity in the interleukin 1 system, followed by an auto-regulatory up-regulation of IL-IRA to reduce the successful binding of interleukin 1 to its receptors. Sjögren's syndrome is also characterized by decreased levels of IL-1 RA in saliva.

In some embodiments, provided herein is a method of treating dermatomyositis using a therapeutically-effective amount of a composition provided herein. Dermatomyositis is a connective-tissue disease related to polymyositis (PM) and Bramaticosis that is characterized by inflammation of the muscles and the skin. The cause is unknown, but it may result from either a viral infection or an autoimmune reaction. Many people diagnosed with dermatomyositis were previously diagnosed with infectious mononucleosis and Epstein-Barr virus. In some cases of dermatomyositis onsets overlaps with other autoimmune diseases.

In some embodiments, provided herein is a method of treating vasculitis using a therapeutically-effective amount of a composition provided herein. Vasculitis refers to a heterogeneous group of disorders that are characterized by inflammatory destruction of blood vessels. Both arteries and veins are affected. Vasculitis is primarily due to leukocyte migration and resultant damage. There are many ways to classify vasculitis. It can be classified by the location of the affected or by the underlying cause. Vasculitides can be classified by the type or size of the blood vessels that they predominantly affect.

In some embodiments, provided herein is a method of treating mixed connective tissue disease (MCTD) using a therapeutically-effective amount of a composition provided herein. MCTD (also known as Sharp's syndrome), is an autoimmune disease, in which the body's defense system attacks itself. It is clinically characterized by presentation with overlapping features of primarily three connective tissue diseases: lupus, scleroderma and polymyositis; and as a result, MCTD is considered an overlap syndrome.

In some embodiments, provided herein is a method of treating tendonitis using a therapeutically-effective amount of a composition provided herein. Tendonitis sometimes called chronic tendinitis, tendinosus, chronic tendinopathy or chronic tendon injury, is damage to a tendon. It is thought to be caused by microtears in the connective tissue in and around the tendon, leading to an increase in tendon repair cells. This may lead to reduced tensile strength, thus increasing the chance of tendon rupture. Classical characteristics of tendinosis include degenerative changes in the collagenous matrix, hypercellularity, hypervascularity and a lack of inflammatory cells.

In some embodiments, provided herein is a method of treating bacterial endocarditis using a therapeutically-effective amount of a composition provided herein. Bacterial endocarditis is a form of endocarditis, or inflammation, of the inner tissue of the heart, such as its valves, caused by infectious agents. The agents are usually bacterial, but other organisms can also be responsible. Historically, infective endocarditis has been clinically divided into acute and subacute presentations. Subacute bacterial endocarditis (SBE) is often due to streptococci of low virulence and mild to moderate illness which progresses slowly over weeks and months and has low propensity to hematogenously seed extracardiac sites. Acute bacterial endocarditis (ABE) is a fulminant illness over days to weeks, and is more likely due to Staphylococcus aureus which has much greater virulence, or disease-producing capacity and frequently causes metastatic infection.

In some embodiments, provided herein is a method of treating psoriasis using a therapeutically-effective amount of a composition provided herein. Psoriasis is common skin disease. It is characterized by cells to building up rapidly on the surface of the skin, forming thick silvery scales and itchy, dry, red patches that are sometimes painful. Psoriasis is a persistent, long-lasting (chronic) disease. You may have periods when your psoriasis symptoms improve or go into remission alternating with times your psoriasis worsens. The cause of psoriasis is not fully understood. There are two main hypotheses about the process that occurs in the development of the disease. The first considers psoriasis as primarily a disorder of excessive growth and reproduction of skin cells. The problem is simply seen as a fault of the epidermis and its keratinocytes. The second hypothesis sees the disease as being an immune-mediated disorder in which the excessive reproduction of skin cells is secondary to factors produced by the immune system. T-cells become active by an unknown mechianum, and then migrate to the dermis where they trigger the release of cytokines such as tumor necrosis factor-alpha TNFα, in particular, This in turn, cause inflammation and the rapid production of skin cells.

In some embodiments, provided herein is a method of treating Rheumatic fever is using a therapeutically-effective amount of a composition provided herein. Rheumatic fever is a systemic inflammatory disease that occurs following a Streptococcus pyogenes infection. It believed to be caused by antibody cross-reactivity that can involve the heart, joints, skin, and brain. This cross-reactivity is a Type II hypersensitivity reaction. Usually, self reactive B-cells remain anergic in the periphery without T-cell costimulation. During a Streptococcus infection, mature antigen presenting cells such as B cells present the bacterial antigen to CD4-T cells which differentiate into helper T2 cells. Helper T2 cells subsequently activate the B cells to become plasma cells and induce the production of antibodies against the cell wall of Streptococcus. However the antibodies may also react against the myocardium and joints producing the symptoms of rheumatic fever.

### Virtual Cultures of Therapies of the Invention

The compositions of the invention were analyzed on a virtual co-culture cell system designed to represent synovium, which can be triggered to represent inflammatory diseases such as Rheumatoid Arthritis (RA) conditions. These experiments have also been validated with in-vivo data as described in the Examples.

The cell systems selected for the virtual co-culture included:
a) macrophages responding to antigens or a bacterial insult thereby promoting an inflammatory response;
b) B-Lymphocytes, which recognize antigens and production of specific antibodies. In an autoimmune inflammatory disease the B-Lymphocytes are responsible for the recognition of self antigens and production of antibodies against them. In Rheumatoid Arthritis (RA), the antibodies against the self antigens are also known as Rheumatoid Factor or RA factor;
c) T-Lymphocytes (CD4+Cells), which recognize antigens presented by the macrophages and B-Lymphocytes and induce an inflammatory cytokine response. In some inflammatory conditions, the interaction of T-Lymphocytes with macrophages and B-Lymphocytes is essential for the initiation of an inflammatory response; and,
d) the bone remodeling system cells (osteoclasts and osteoblasts) were selected to simulate their effect on the bone in response to the inflammatory cytokines.

The interaction of T-Lymphocytes with macrophages and B-Lymphocytes is an essential step for the initiation of a fully fledged inflammatory response.

In these virtual experiments, the system was first stimulated with high doses of antigen and then cultured for a minimum of about 18 hours. The culture time was selected to allow the system to attain severe RA conditions through all inflammatory mediators like cytokines, chemokines, and prostaglandins. After about 18 hours of antigen stimulation, an RA synovium-like environment was created, where the effect of the inflammatory cells (i.e. macrophages, T-Lymphocytes and B Lymphocytes) could be analyzed for mediating a localized inflammation and modulating bone destruction.

The drug compounds CW299, CW304, CW330 were then administered concomitantly into the RA cell co-culture system and the cells were cultured for a minimum of about 12 hours. The drug administration was performed at multiple dosage ratios for the three drug molecules.

The effect of the multiple dosage ratios was evaluated after about a minimum of 12 hours of culture by assaying the extent of decrease/increase in the cytokine milieu responsible for joints swelling and tendering.

The major cytokines assayed included TNFα, IL6, IL1β, IL17 and CCL2. These cytokines are mainly responsible for joint swelling, the other proteins like Matrix Metalloproteinases (e.g. MMP9), Cathepsin K and other osteoclastogenesis inducing factor like RANKL and many other bio-marker levels are assayed to determine their effect.

Other vital biomarkers like Prostaglandins (e.g. Prostaglandin E2) and C-Reactive Proteins (CRP) were assayed to estimate the levels of inflammation in the synovium. Based on the assayed biomarkers, an ACR score was calculated using the ACR calculation criteria published by the American College of Rheumatology in 2010.

Drug CW299 impacts multiple cell types including the Macrophage, B-Lymphocyte, T-Lymphocyte, Mast cells, Synovial Fibroblast, Osteoclast and Osteoblast functions by adversely affecting the macrophage colony stimulating factor (CSF1), platelet derived growth factor (PDGFR), T-cell response (TCR) and B-cell response (BCR) pathways. These are the key pathways involved in pro-inflammatory stimulation of these cell types.

Drug CW304 targets the enzyme phosphodiesterase 4 (PDE4) and induces anti-inflammatory effects by increasing the levels of cyclic AMP (cAMP) in different cell systems.

Drug CW330 is an inhibitor which blocks the activation of angiotensin II AT1 receptors or which inhibits the formation of Angiotensin II from Angiotensin I by blocking Angiotensin Converting Enzyme (ACE). The activated receptor in turn couples to Gq/11 and thus activates phospholipase C and increases the cytosolic Ca2+ concentrations, which in turn triggers cellular responses such as stimulation of protein kinase C. Activated receptor also inhibits adenylate cyclase and activates various tyrosine kinases.

All three drugs through very different mechanisms of action have converging antagonistic effects on major pro-inflammatory transcription factors such as NFKB, API and others and are responsible for causing a systemic reduction in the pro-inflammatory cytokines and chemokines that are involved in disease physiology and progression.

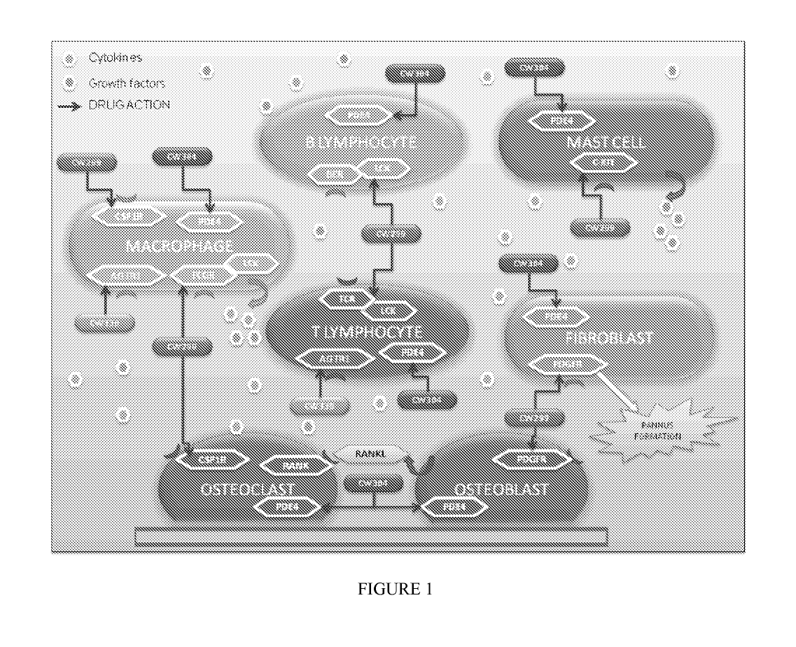

As used in the virtual co-cultures, and throughout FIGS. 1-63, 65, 67, 69, 71-73, 75, 77-79, 81, 83, and 85-105 and references to the same: CW299 is imatinib mesylate; CW304 is roflumilast; CW330 is fosinopril; CW299304 is a combination of imatinib mesylate and roflumilast; CW299330 is a combination of imatinib mesylate and fosinopril; CW330304 is a combination of fosinopril and roflumilast; and CW299330304 is a combination of imatinib, fosinopril, and roflumilast. The Figures reflect the simulated dosing for the virtual co-cultures.

The drug compounds CW299, CW304, and CW330 were administered concomitantly to the RA cell virtual co-culture system, and the cells were cultured for a minimum of about 12 hours. The drug administration was performed at multiple dosage ratios across an array of samples for each drugs. The effect of the multiple dosage ratios was evaluated after about 12 hours of culture by assaying the extent of decrease/increase in the cytokine population responsible for the swelling and tendering of joints. The major cytokines assayed included TNFα, IL6, IL1β, IL17 and CCL2, which are responsible for joint swelling. Other proteins including matrix metalloproteinases (e.g. MMP9), cathepsin K, and other osteoclastogenesis-inducing factors, such as RANK ligand, were assayed to determine their effect. Other vital biomarkers including Prostaglandins (e.g. Prostaglandin E2) and C-Reactive Proteins (CRP) were assayed to estimate the levels of inflammation in the synovium.

Based on the assayed biomarkers, an ACR score was calculated using the ACR calculation criteria published by the American College of Rheumatology in 2010. See ARTHRITIS & RHEUMATISM; Vol. 62, No. 9, September 2010, pp 2569-2581, American College of Rheumatology, which is incorporated by reference herein in its entirety.

“ACR score” is a scale to measure change in rheumatoid arthritis symptoms. It is named after the American College of Rheumatology. Different degrees of improvement are referred to as ACR20 (low), ACR50 (moderate), ACR70 (high). A level of improvement less than ACR20 is identified, “resist”. ACR50 response—which includes reducing the signs and symptoms of disease by 50%, according to criteria established by the American College of Rheumatology (ACR). The ACR score allows a ‘common standard’ between researchers.

The drugs were also tested on a TNF resistant (anti-TNF non responders) cell co-culture system. This system was designed by desensitizing the TNF receptors by about 80% on all the cells in the co-culture system. The co-culture system was also populated with about 5 fold more B-Lymphocytes compared to the system described above. T-Lymphocyte co-stimulation was also enhanced by about 4 folds. Otherwise, the experiment followed the protocol described above.

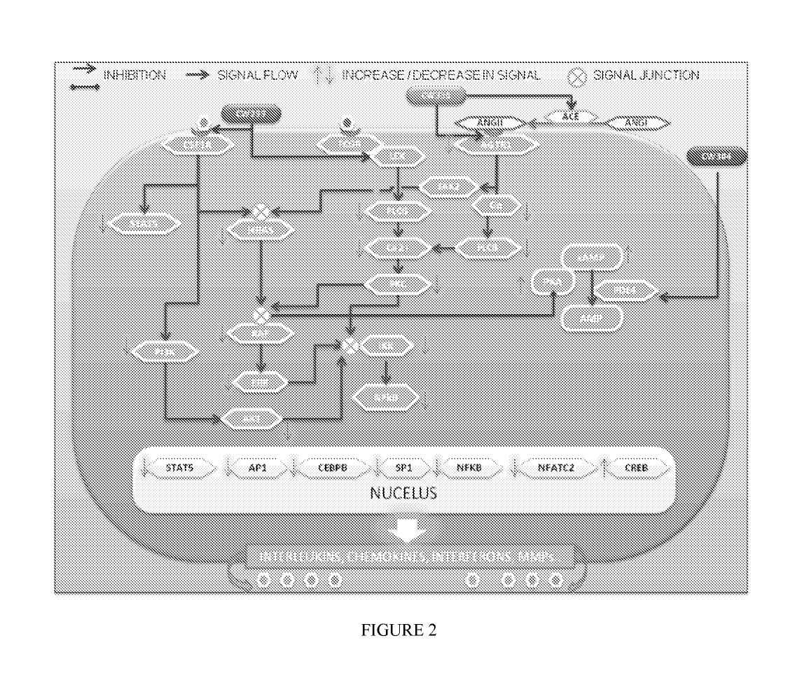

The combinations of compounds described herein can provide therapeutic benefits at low dosage, including synergistic benefits. FIGS. 1 and 2 illustrate examples of pathways associated with the compounds of the present disclosure, along with illustration of the biochemical targets implicated in the relevant inflammatory pathways.

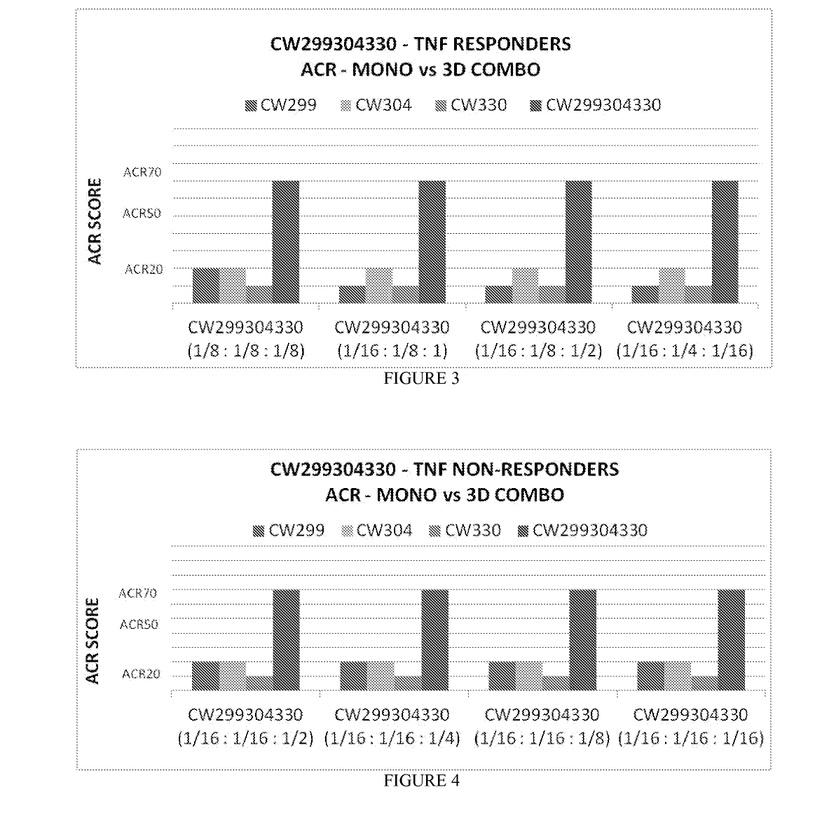

FIG. 3 illustrates the individual drugs and the combination drug efficacy in terms of ACR (American College of Rheumatology) Score in TNF responders. The bars represent the efficacy of individual drugs CW299, CW304, CW330 and the combination thereof. The various ratios used were: (a) ⅛:⅛:⅛, (b) 1/16:⅛:1, (c) 1/16:⅛:½ and (d) 1/16: ¼: 1/16.

FIG. 4 illustrates the individual drugs and the combination drug efficacy in terms of ACR (American College of Rheumatology) score in a TNF-resistive system (anti-TNF non-responders). The bars represent the efficacy of individual drugs CW299, CW304, CW330 and the combination thereof. From this it evident that the given drug combination works in both types of systems. The various ratios used were: (a) 1/16: 1/16:½, (b) 1/16: 1/16:¼, (c) 1/16: 1/16:⅛, (d) 1/16: 1/16: 1/16.

The results illustrated in FIG. 3 and FIG. 4 indicate that CW299304330 exhibits better efficacy than CW299, CW304, and CW330 individually, as CW299304330 has a much higher ACR score than the individual drugs.

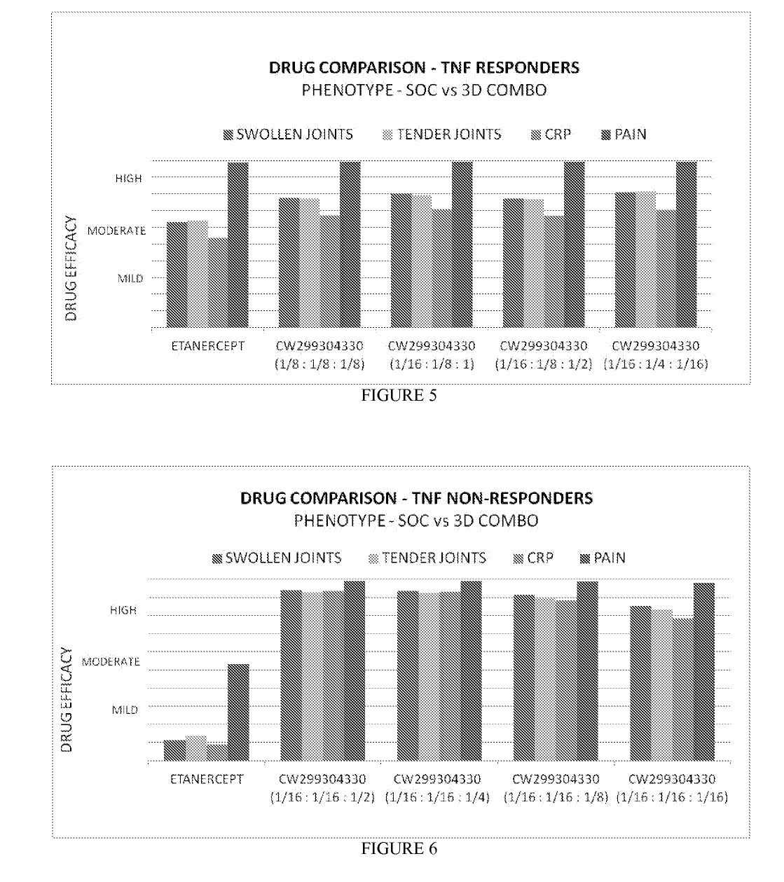

FIG. 5 compares the efficacies of individual drugs and combinations thereof of the invention with Etanercept (ENBREL®). The comparison was done in TNF responders across clinically measurable parameters including swollen joints, tender joints, CRP, and pain. The various ratios of the combination of compounds used for the comparison were: (a) ⅛:⅛:⅛, (b) 1/16:⅛:1, (c) 1/16:⅛:½, and (d) 1/16: ¼: 1/16.

FIG. 6 compares the efficacies of the combinations in a TNF non-responder system of the invention with Etanercept (ENBREL®). The comparison was done across clinically measurable parameters including swollen joints, tender joints, CRP, and pain. The various ratios of the combination of compounds used for the comparison were: (a) 1/16: 1/16:½, (b) 1/16: 1/16:¼, (c) 1/16: 1/16:⅛, and (d) 1/16: 1/16: 1/16.

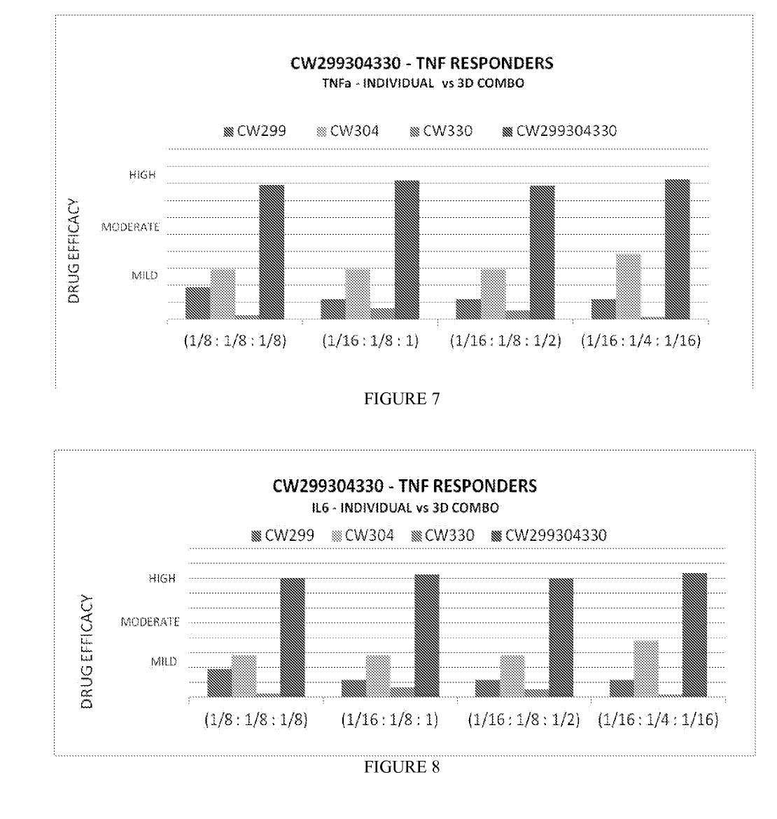

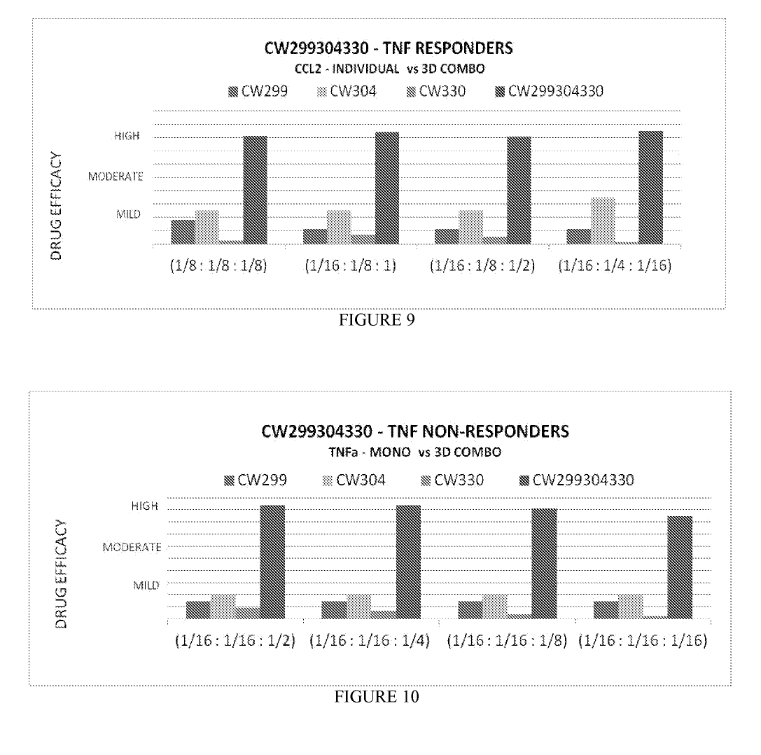

FIGS. 7, 8, and 9 show the efficacy data with regards to TNF, IL6, and CCL2 biomarkers in TNF responders for the three individual drugs CW299, CW304, CW330 and the combination thereof. The various ratios of the combination of compounds used for the comparison were: (a) ⅛:⅛:⅛, (b) 1/16:⅛:1, (c) 1/16:⅛:½ and (d) 1/16:¼: 1/16.

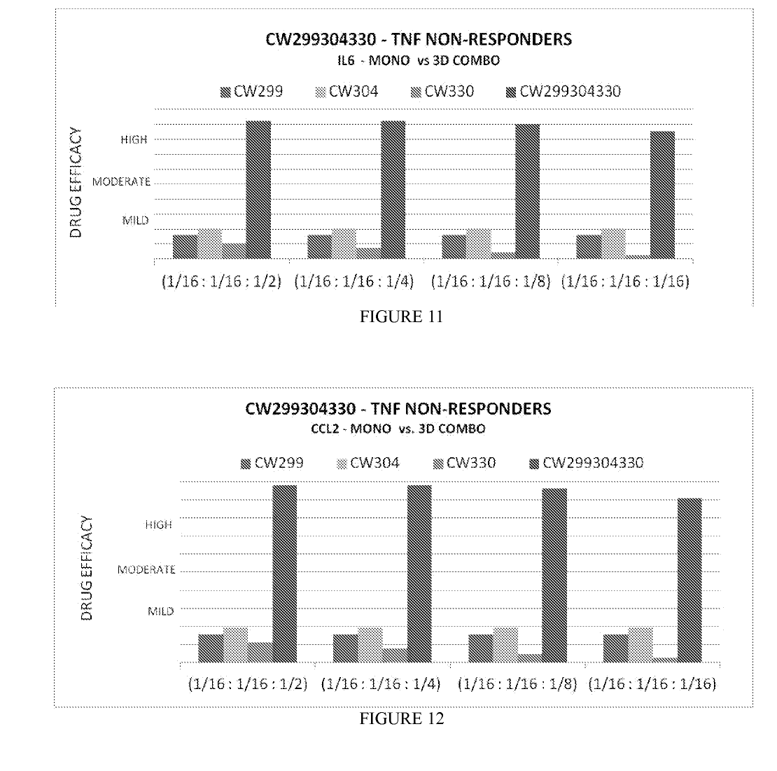

FIGS. 10, 11, and 12 show efficacy data with regards to TNF, IL6, CCL2 biomarkers in TNF non-responders for the three individual drugs CW299, CW304, CW330 and the combination thereof. The various ratios of the combination of compounds used for the comparison were: (a) 1/16: 1/16:½, (b) 1/16: 1/16:¼, (c) 1/16: 1/16:⅛, and (d) 1/16: 1/16: 1/16.

The results illustrated in FIGS. 7, 8, 9, 10, 11, and 12 indicate that the three drug combination CW299304330 exhibited better efficacy than the individual drugs CW299, CW304 and CW330, both in TNF responders as well as TNF non-responders.

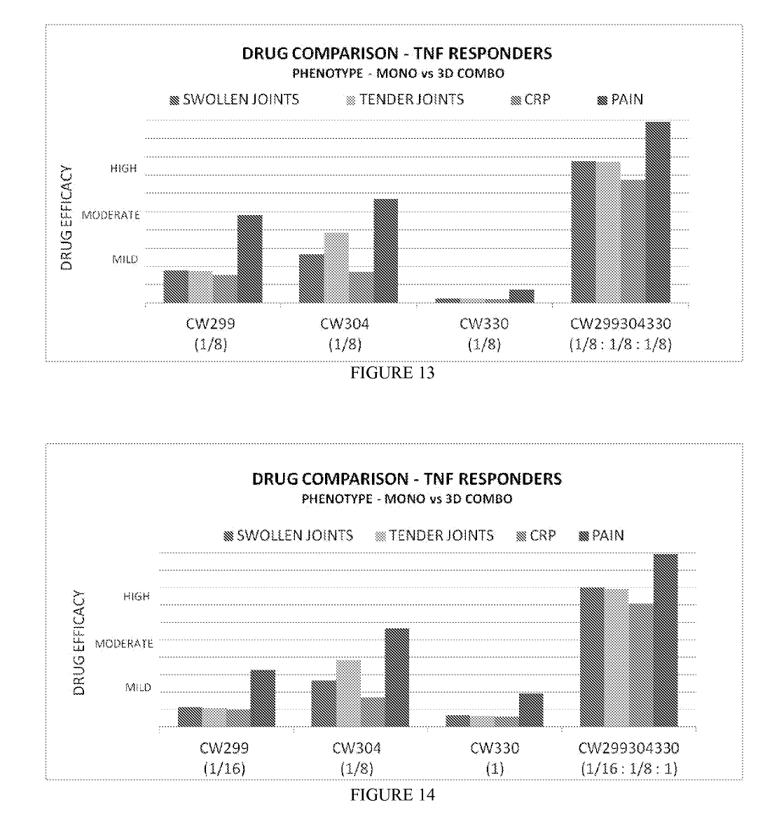

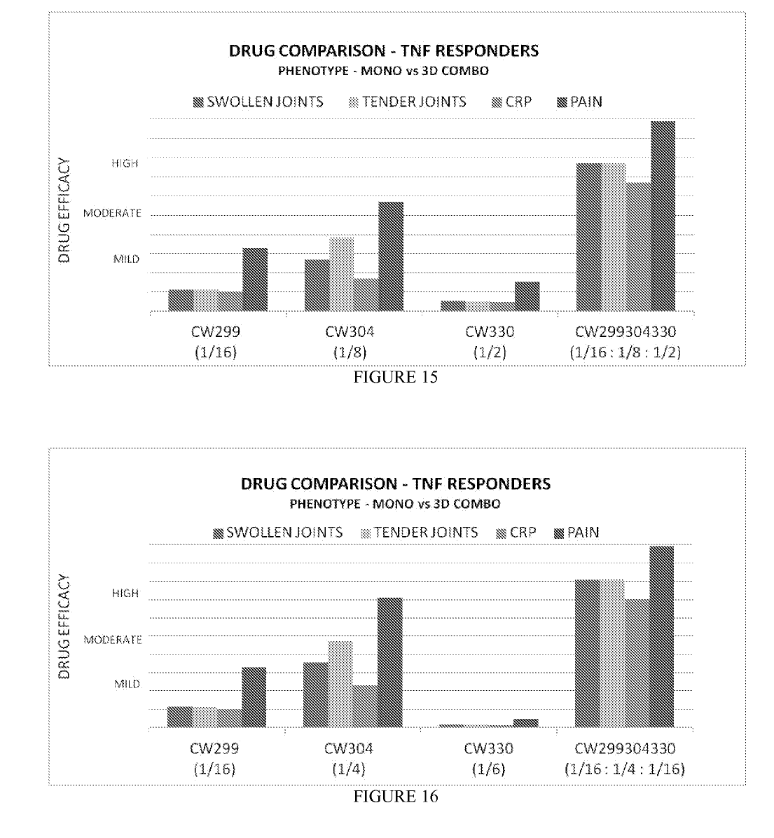

FIGS. 13, 14, 15, and 16 compare the efficacy of the individual drugs CW299, CW304, and CW330 with a combination of these three drugs at the same dosage.

The comparison was done across clinically measurable parameters including swollen joints, tender Joints, CRP, and pain in TNF responders. The various ratios of the combination of compounds used for the comparison were: (a) ⅛:⅛:⅛, (b) 1/16:⅛:1, (c) 1/16:⅛:½, and (d) 1/16:¼: 1/16, which corresponds to FIGS. 13, 14, 15, and 16 respectively.

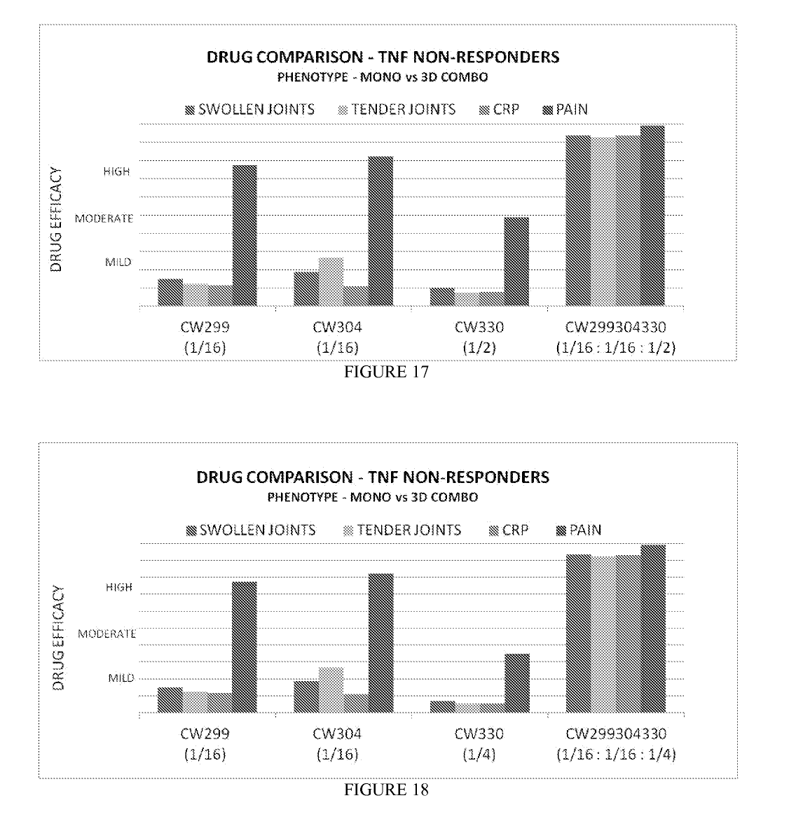

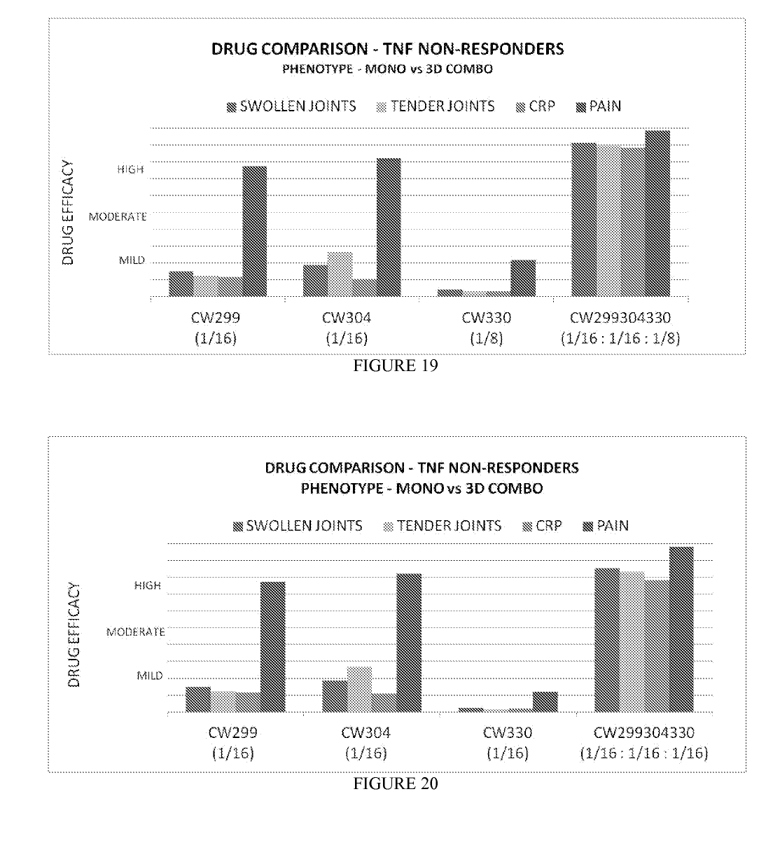

FIGS. 17, 18, 19, and 20 compare the efficacy of the individual drugs CW299, CW304, and CW330 with a combination of these three drugs at the same dosage. The comparison was done across clinically measurable parameters including swollen joints, tender joints, CRP, and pain in a TNF non-responders (anti-TNF drug non-responders) system. The various ratios of the combination of compounds used for the comparison were: (a) 1/16: 1/16:½, (b) 1/16: 1/16:¼, (c) 1/16: 1/16:⅛, and (d) 1/16: 1/16: 1/16, which correspond to FIGS. 17, 18, 19, and 20 respectively.

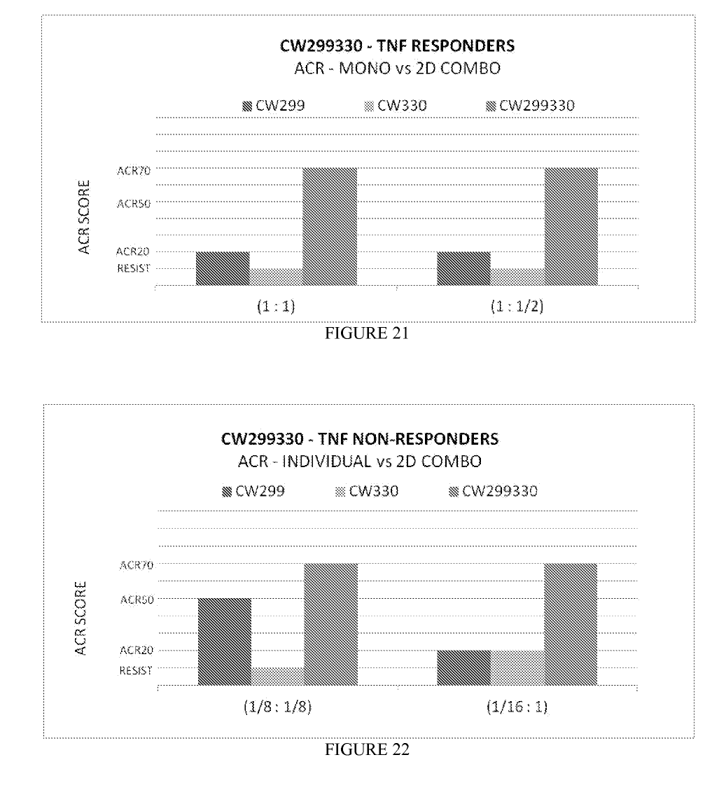

FIG. 21 illustrates efficacy data of the individual drugs CW299 and CW330 and combinations thereof in terms of ACR Score in TNF responders. The first two bars of FIG. 21 represent the efficacy of individual drugs CW299 and CW330, whereas the third bar represents the efficacy of the combination in the same dosage of CW299 and CW330.

The various ratios of the combination of compounds used for the comparison were: (a) 1:1 and (b) 1:½.

FIG. 22 illustrates efficacy data of the individual drugs CW299 and CW330 and its combination drug in terms of ACR Score in a TNF-resistive system (anti-TNF non-responders). The first two bars of FIG. 22 represent the efficacy of individual drugs CW299 and CW330, whereas the third bar represents the efficacy of the combination in the same dosage of CW299 and CW330. The various ratios of the combination of compounds used for the comparison were: (a) ⅛:⅛ and (b) 1/16:1.

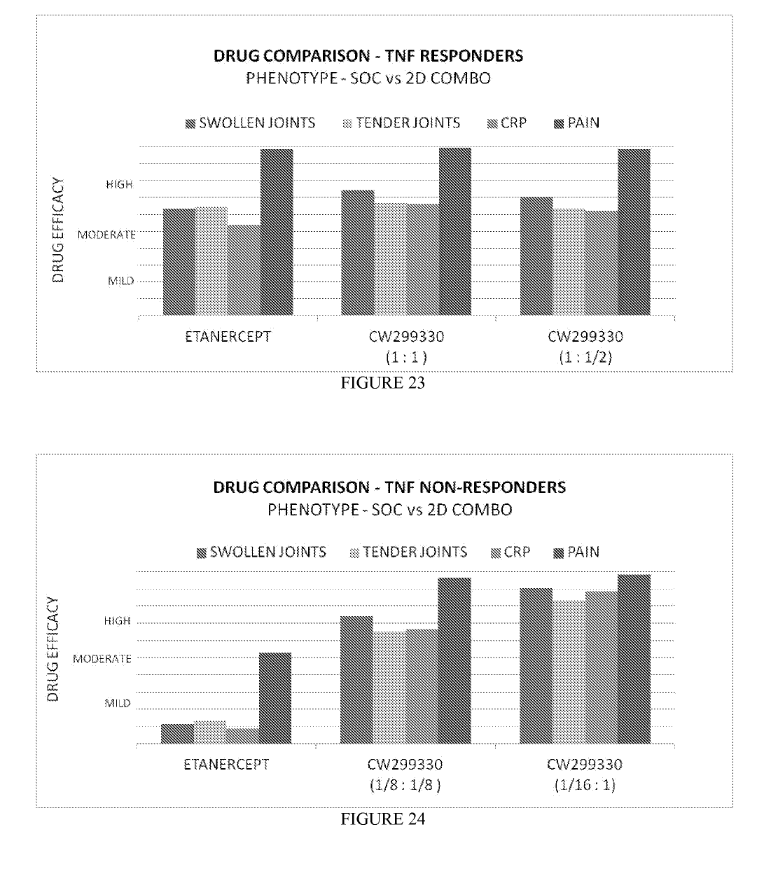

FIG. 23 compares the efficacy of a combination of the drugs CW299 and CW330 with Etanercept (ENBREL®), in TNF responders. The comparison was done across clinically-measurable parameters including swollen joints, tender joints, CRP, and pain. The various ratios of the combination of compounds used for the comparison were: (a) 1:1 and (b) 1:½.

FIG. 24 compares the efficacy of the combination of the drugs CW299 and CW330 with Etanercept (ENBREL®), in a TNF-resistive system (anti-TNF non-responders).

The comparison was done across clinically-measurable parameters like swollen joints, tender Joints, CRP, and pain. The various ratios of the combination of compounds used for the comparison were: (a) ⅛:⅛ and (b) 1/16:1.

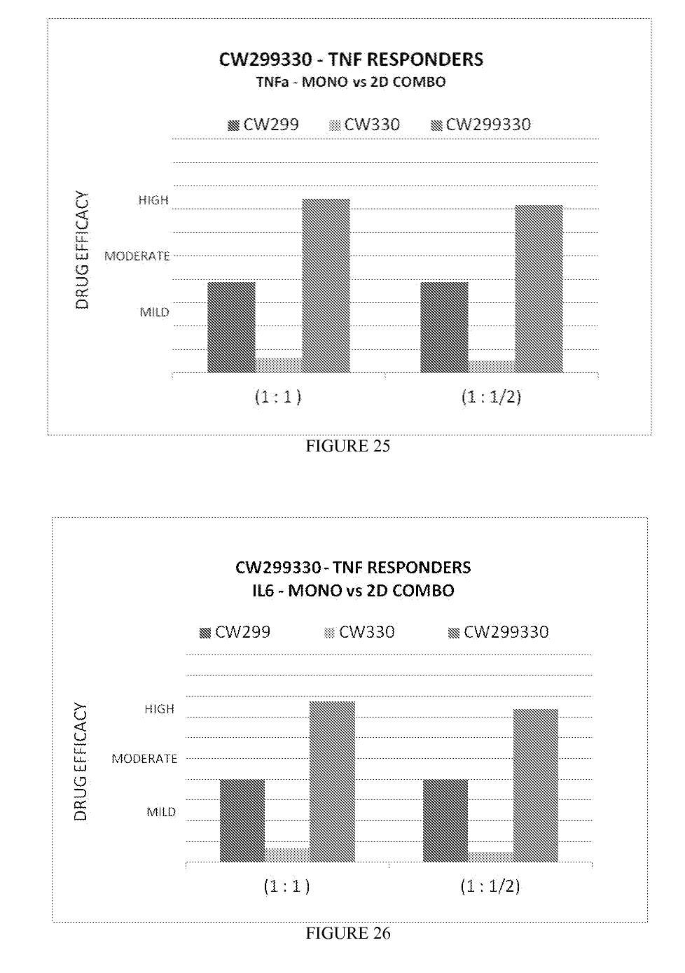

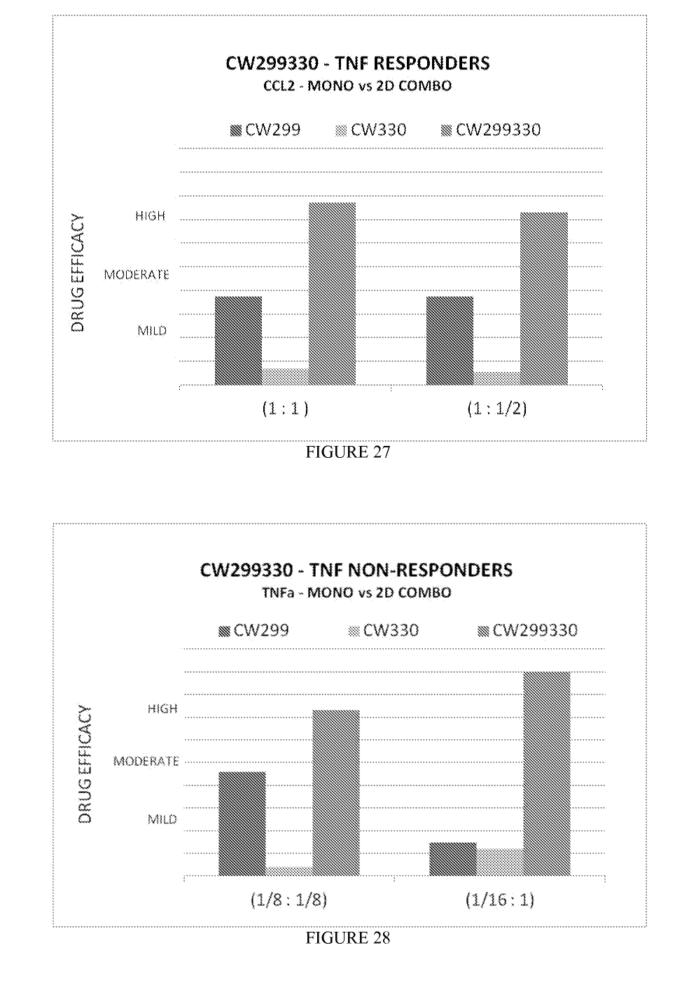

FIGS. 25, 26, and 27 compare the efficacy of individual drugs CW299 and CW330 and a combination thereof with regards to TNF, IL6, and CCL2 biomarkers in TNF responders, respectively. The various ratios of the combination of compounds used for the comparison were: (a) 1:1 and (b) 1:½.

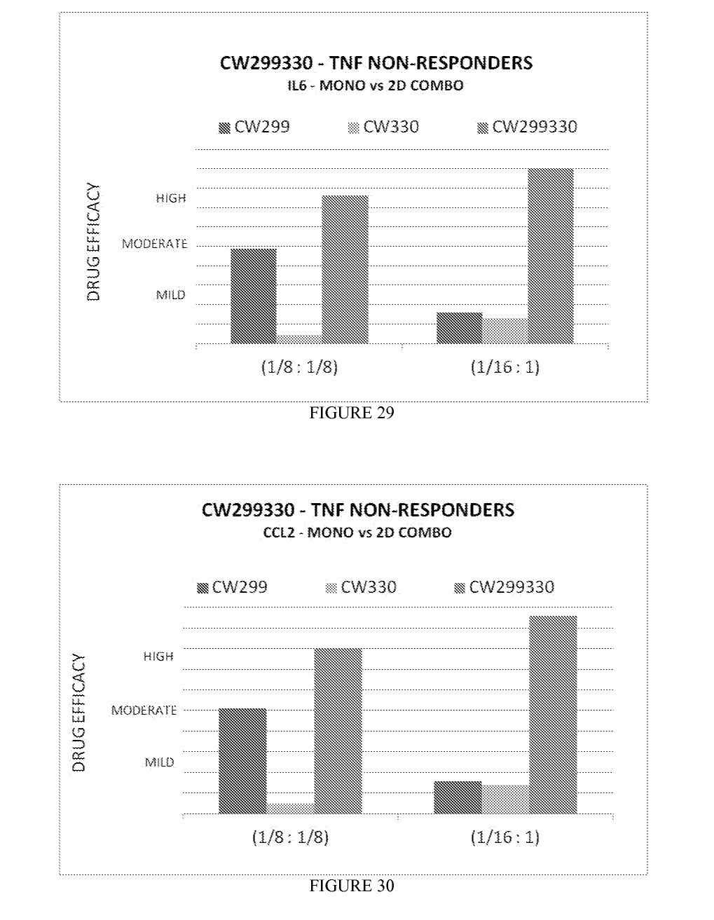

FIGS. 28, 29, and 30 compare the efficacy of individual drugs CW299 and CW330 and a combination thereof with regards to TNF, IL6, and CCL2 biomarkers in a TNF resistive system (anti-TNF non-responders), respectively. The various ratios of the combination of compounds used for the comparison were: (a) ⅛:⅛ and (b) 1/16:1.

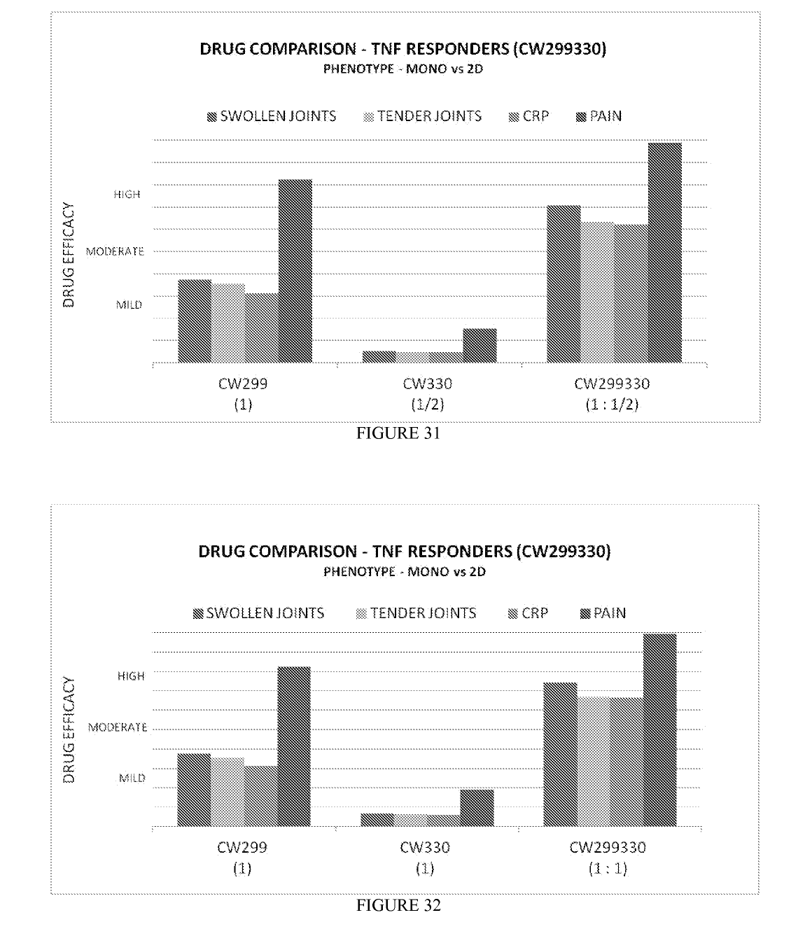

FIGS. 31 and 32 compare the efficacy of the individual drugs CW299 and CW330 with a combination of these two drugs at same dosage. The comparison was done across clinically-measurable parameters including swollen joints, tender Joints, CRP, and pain in TNF responders. The various ratios of the combination of compounds used for the comparison were: (a) 1:1 and (b) 1:½, which correspond to the FIGS. 31 and 32, respectively.

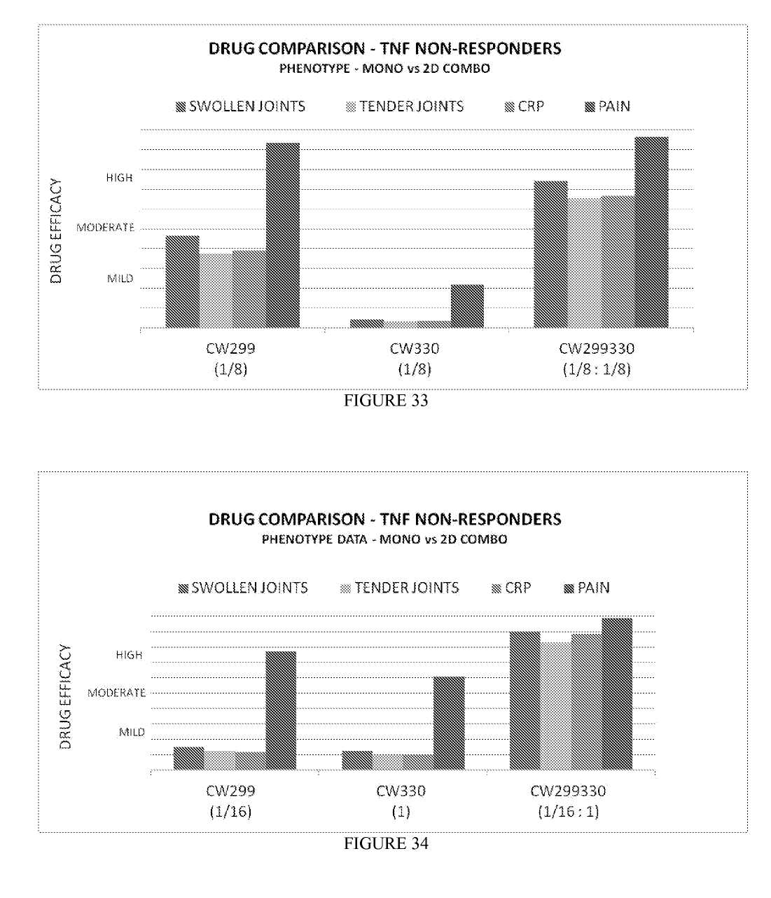

FIGS. 33 and 34 compare the efficacy of the individual drugs CW299 and CW330 with a combination of these two drugs at same dosage. The comparison was done across clinically-measured parameters like swollen joints, tender joints, CRP, and pain in TNF-resistant (anti-TNF non-responsive) system. The various ratios of the combination of compounds used for the comparison were: (a) ⅛:⅛ and (b) 1/16:1, which correspond to the FIGS. 33 and 34, respectively.

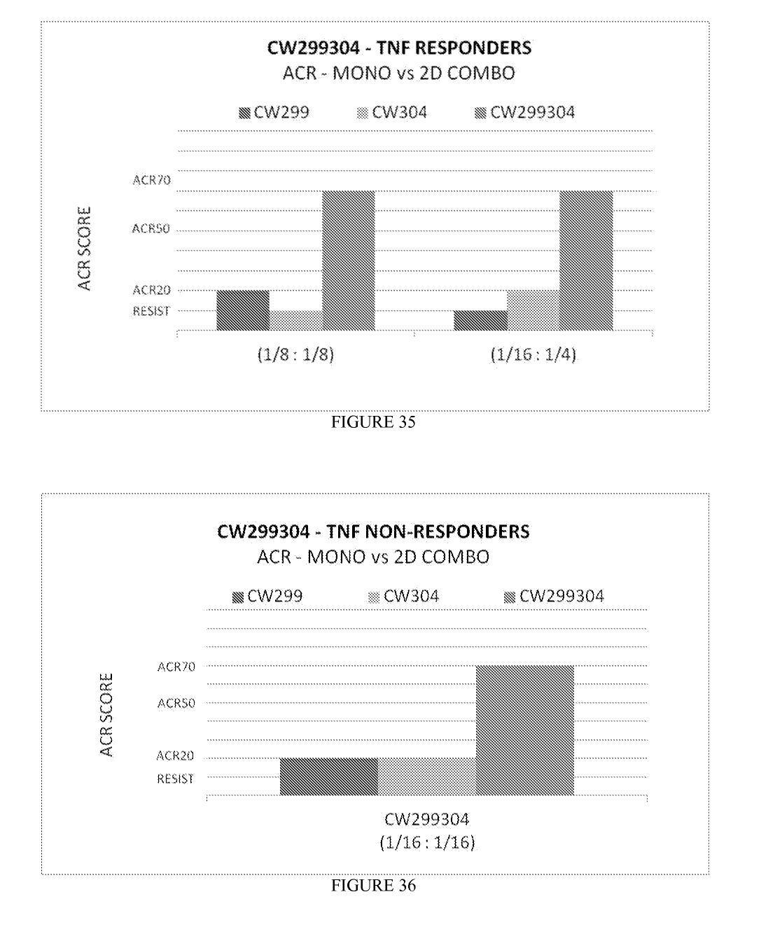

FIG. 35 compares efficacy data of the individual drugs CW299 and CW304 and the combination thereof in terms of ACR Score in TNF responders. The first two bars of FIG. 35 represent the efficacy of individual drugs CW299 and CW304, whereas the third bar represents the efficacy of the combination in the same dosage of CW299 and CW304.

The various ratios of the combination of compounds used for the comparison were: (a) ⅛:⅛ and (b) 1/16:¼.

FIG. 36 compares efficacy data of the individual drugs CW299 and CW304 and the combination thereof in terms of ACR Score in TNF resistive system (anti-TNF non-responders). The first two bars of FIG. 36 represent the efficacy of individual drugs 1/16 of CW299 and 1/16 of CW304, whereas the third bar represents the efficacy of the combination of the two drugs, which had dosages 1/16 of CW299 and 1/16 of CW304.

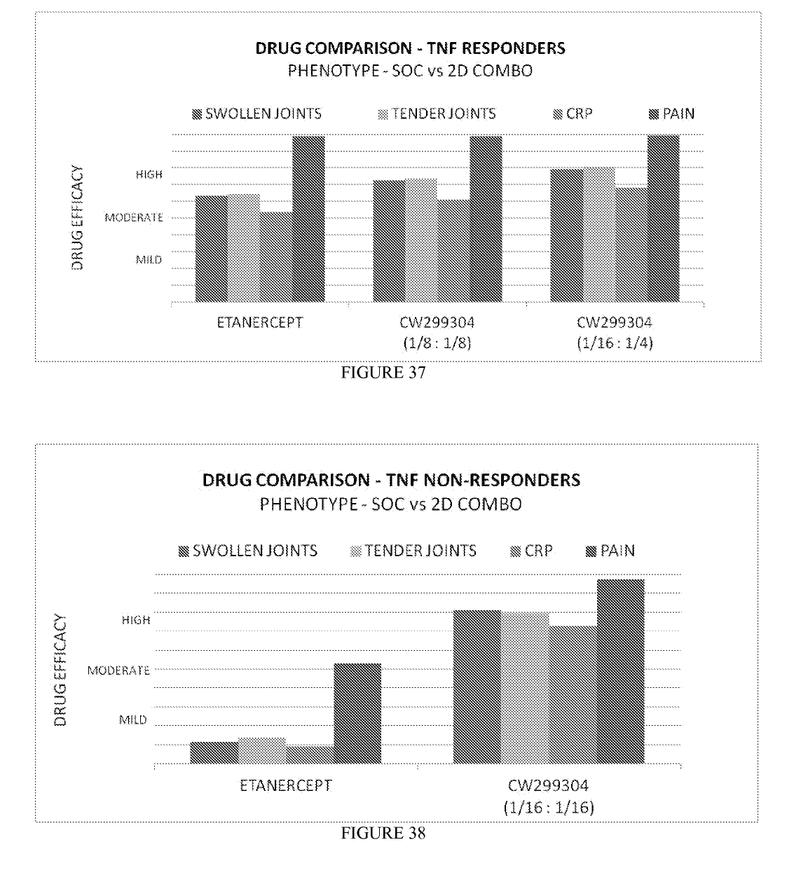

FIG. 37 compares the efficacy of a combination of the drugs CW299 and CW304 with Etanercept (ENBREL®) in TNF responders. The comparison was done across clinically-measurable parameters including swollen joints, tender joints, CRP, and pain. The various ratios of the combination of compounds used for the comparison were: (a) ⅛:⅛ and (b) 1/16:¼.

FIG. 38 compares the efficacy of the combination of a 1/16 dose of CW299 and a 1/16 dose of CW304 with Etanercept (ENBREL®) in a TNF-resistive system (anti-TNF non-responders). The comparison was done across clinically-measurable parameters including swollen joints, tender Joints, CRP, and pain.

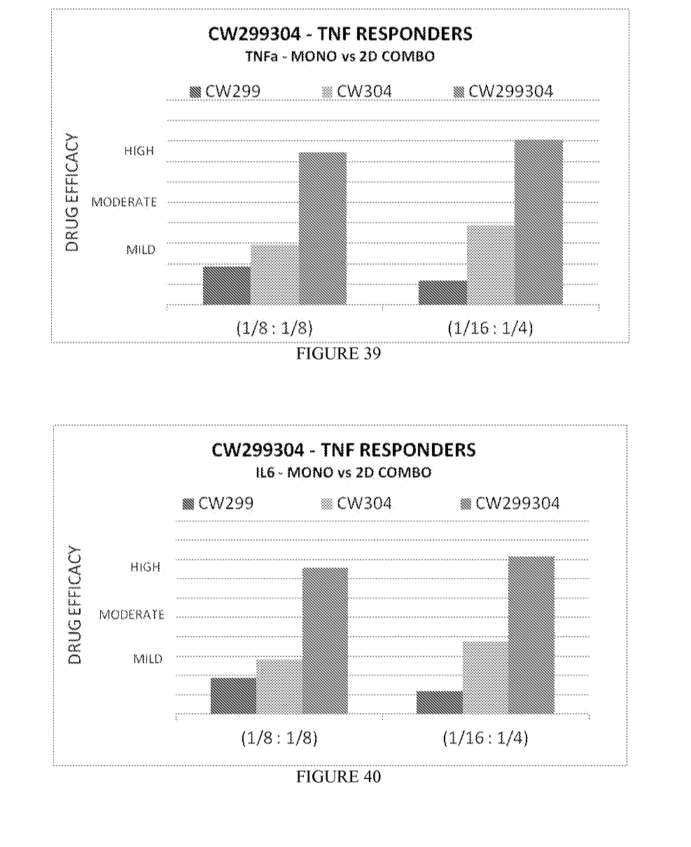

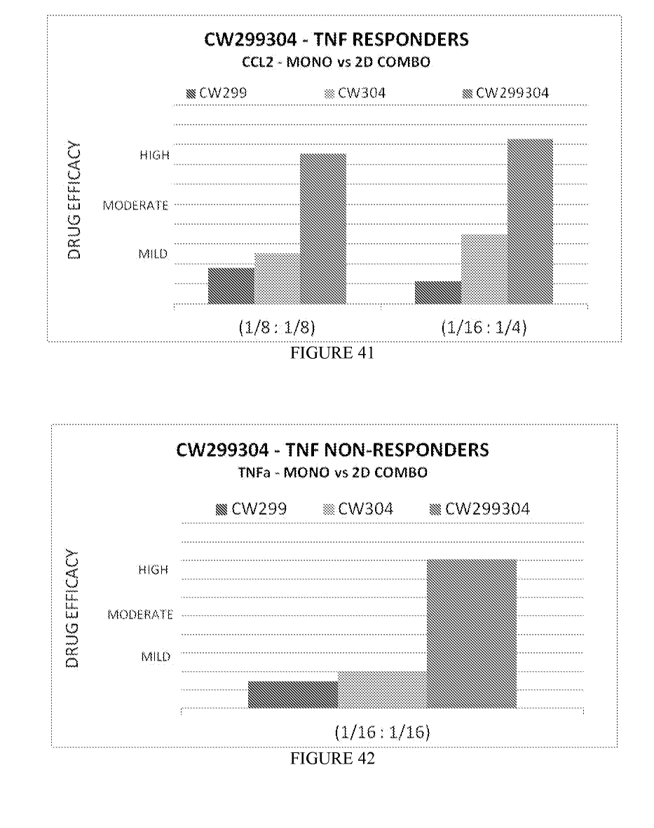

FIGS. 39, 40, and 41 compare the efficacy of individual drugs CW299 and CW304 and the combination thereof with regards to TNF, IL6, and CCL2 biomarkers in TNF responders, respectively. The various ratios of the combination of compounds used for the comparison were: a) ⅛:⅛ and (b) 1/16:¼.

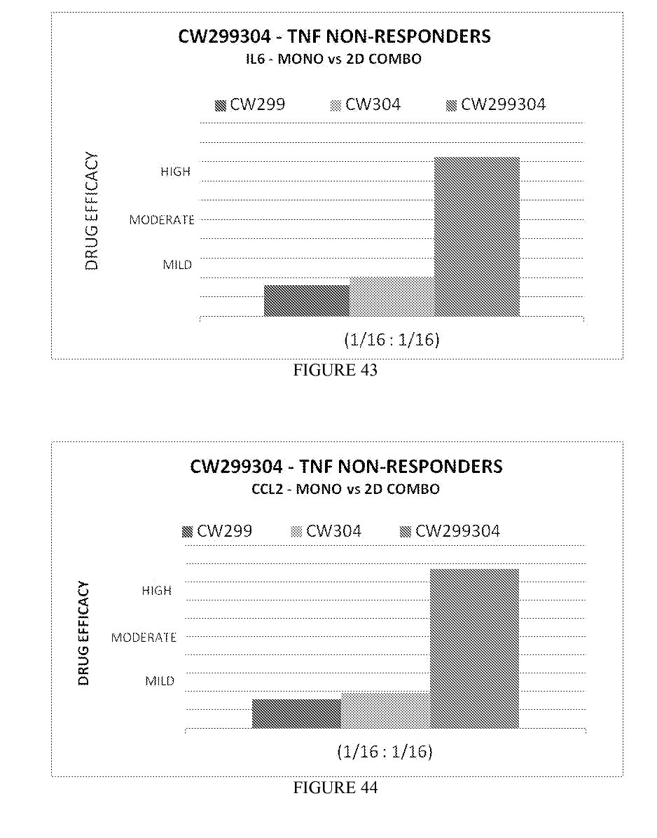

FIGS. 42, 43, and 44 compare the efficacy of individual drugs and combinations thereof with regards to IL6, TNF, and CCL2 biomarkers in a TNF-resistive system (anti-TNF non-responders), respectively. The various ratios of the combination of compounds used for the comparison were: 1/16 dosage of CW299, 1/16 dosage of CW304 and CW299304, which is a combination of 1/16 dosage of CW299 and 1/16 dosage of CW304.

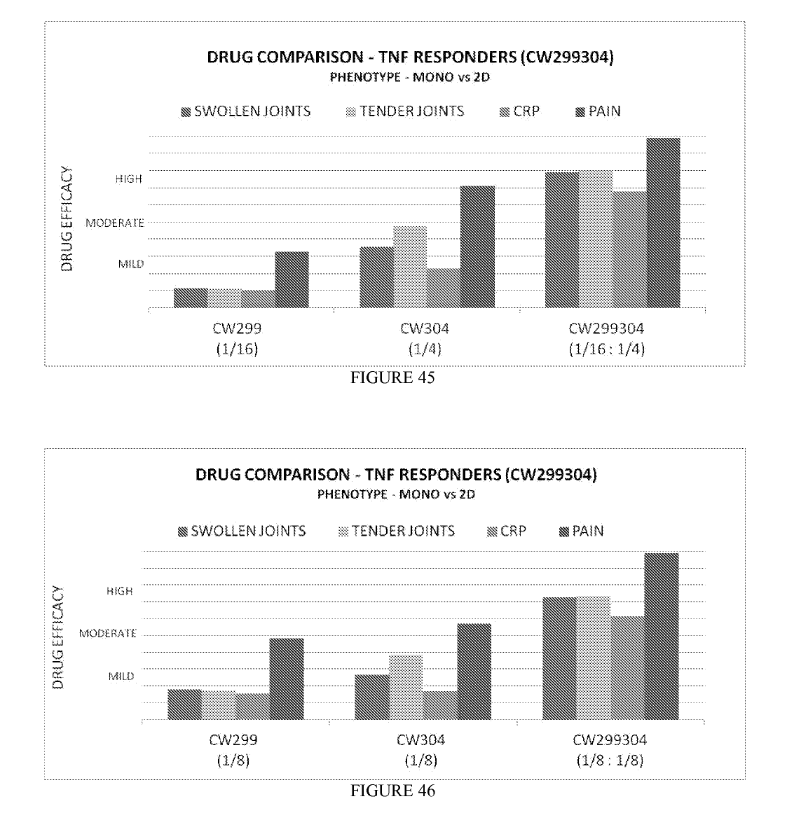

FIGS. 45 and 46 compare the efficacy of the individual drugs CW299 and CW304 with a combination thereof at the same dosage. The comparison was done across clinically-measurable parameters including swollen joints, tender joints, CRP, and pain in TNF responders. The various ratios of the combination of compounds used for the comparison were: (a) ⅛:⅛ and (b) 1/16:1 which correspond to FIGS. 45 and 46, respectively.

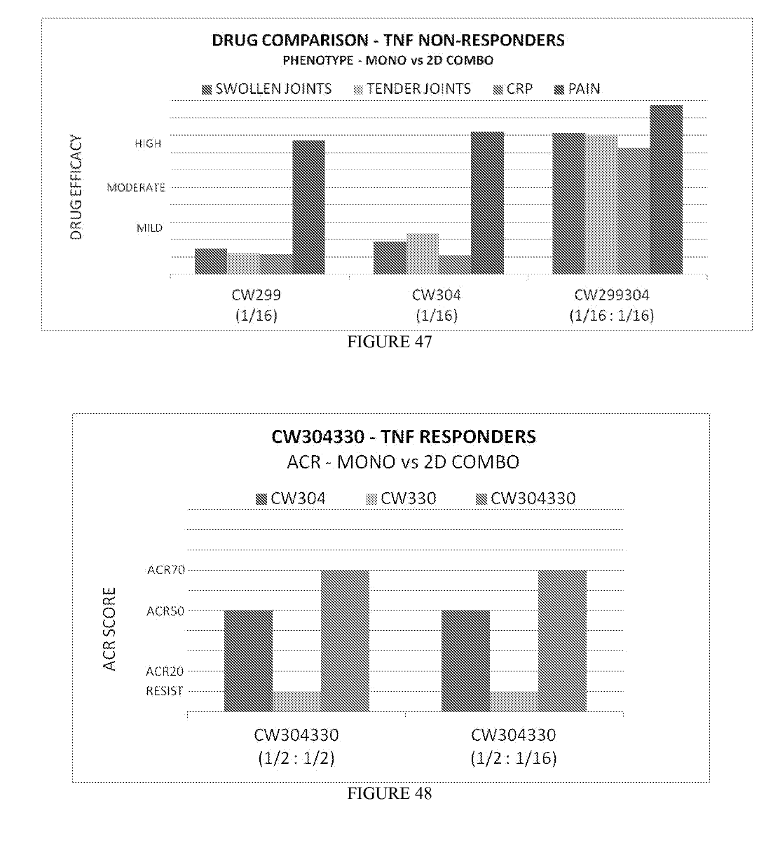

FIG. 47 compares the efficacy of a 1/16 dosage of CW299, a 1/16 dosage of CW304, and CW299304, which is a combination of 1/16 dosage of CW299 and 1/16 dosage of CW304. The comparison was done across clinically-measured parameters including swollen joints, tender joints, CRP, and pain in a TNF-resistant (anti-TNF non-responsive) system.

FIG. 48 illustrates efficacy data of the individual drugs CW304 and CW330 and the combination thereof in terms of ACR Score in TNF responders. The first two bars of FIG. 48 represent the efficacy of individual drugs CW304 and CW330, whereas the third bar represents the efficacy of the combination in the same dosage of CW304 and CW330.

The various ratios of the combination of compounds used for the comparison were: (a) ½:½ and (b) ½: 1/16.

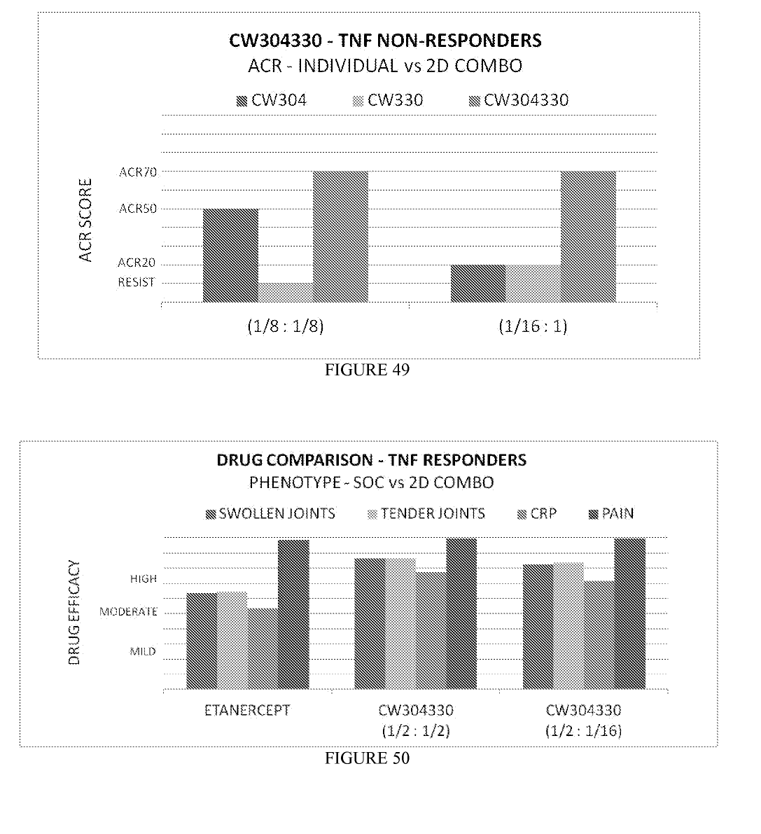

FIG. 49 illustrates efficacy data of the individual drugs CW304 and CW330 and the combination thereof in terms of ACR Score in a TNF-resistive system (anti-TNF non-responders). The first two bars of FIG. 49 represent the efficacy of individual drugs CW304 and CW330, whereas the third bar represents the efficacy of the combination in the same dosage of CW304 and CW330. The various ratios of the combination of compounds used for the comparison were: (a) ⅛:⅛ and (b) 1/16:1.

FIG. 50 compares the efficacy of a combination of the drugs CW304, CW330 with Etanercept (ENBREL®) in TNF responders. The comparison was done across clinically-measurable parameters like swollen joints, tender joints, CRP, and pain. The various ratios of the combination of compounds used for the comparison were: (a) ½:½ and (b) ½: 1/16.

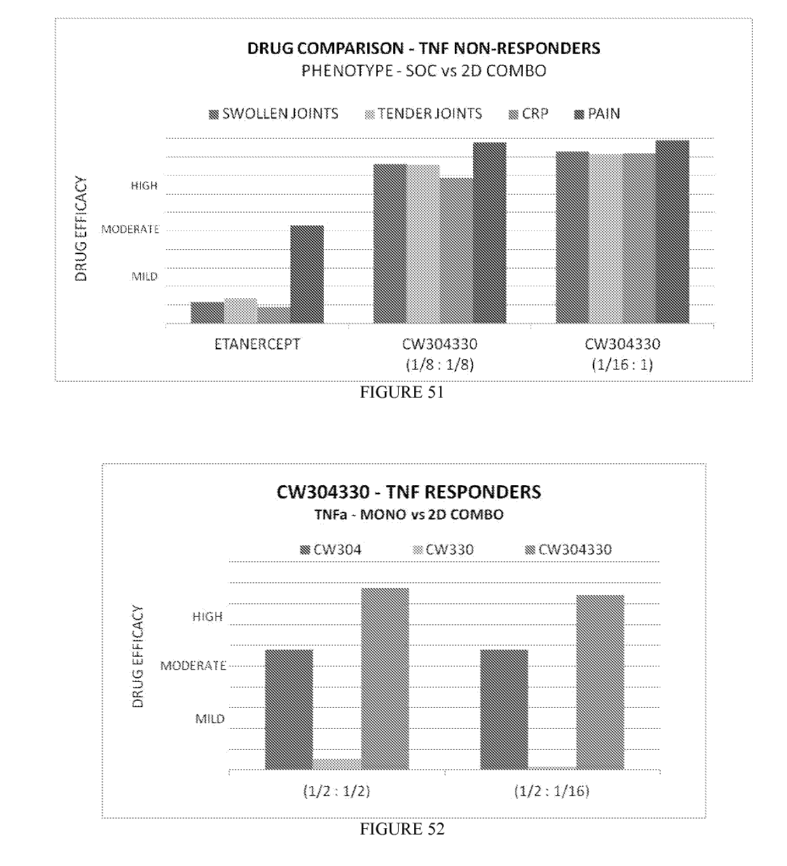

FIG. 51 compares the efficacy of the combination of the drugs CW304 and CW330 with Etanercept (ENBREL®) in a TNF-resistive system (anti-TNF non-responders).

The comparison was done across clinically-measurable parameters like swollen joints, tender joints, CRP, and pain. The various ratios of the combination of compounds used for the comparison were: a) ⅛:⅛ and (b) 1/16:1.

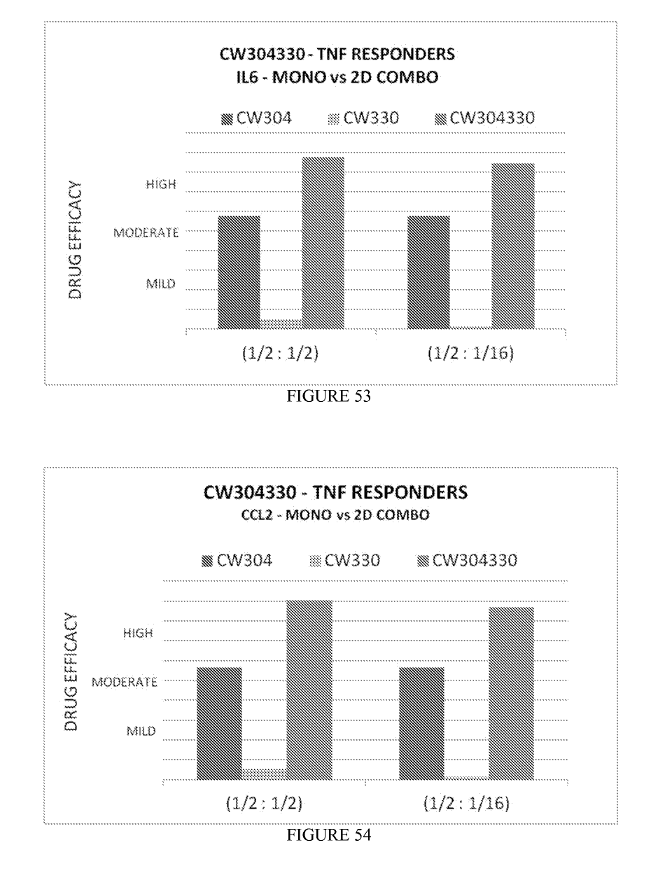

FIGS. 52, 53, and 54 compare the efficacy of individual drugs CW304 and CW330 and the combination thereof with regards to TNF, IL6, and CCL2 biomarkers in TNF responders, respectively. The various ratios of the combination of compounds used for the comparison were: (a) ½:½ and (b) ½: 1/16.

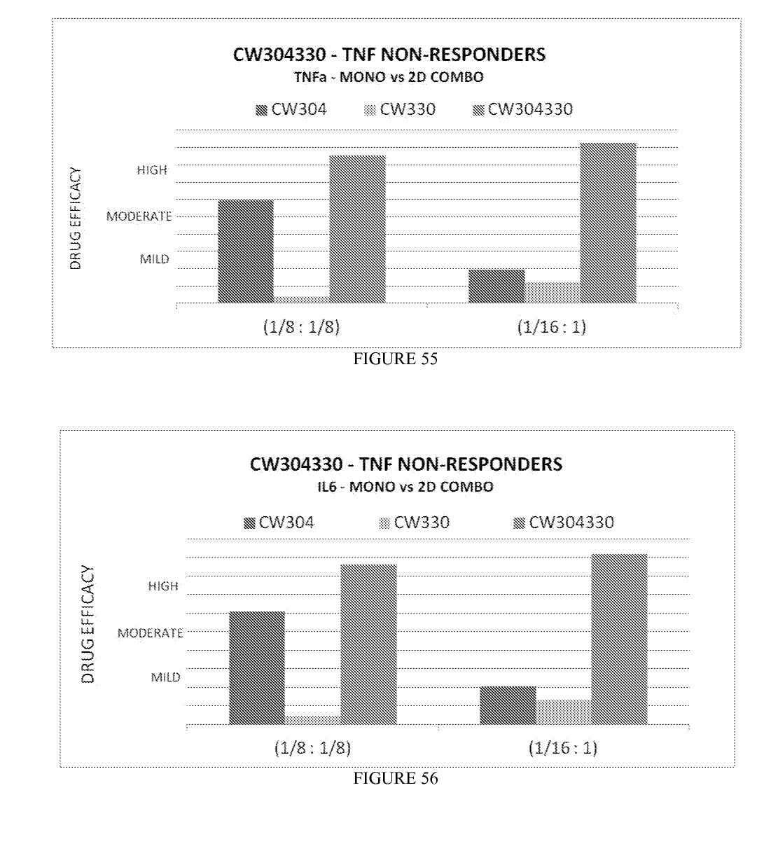

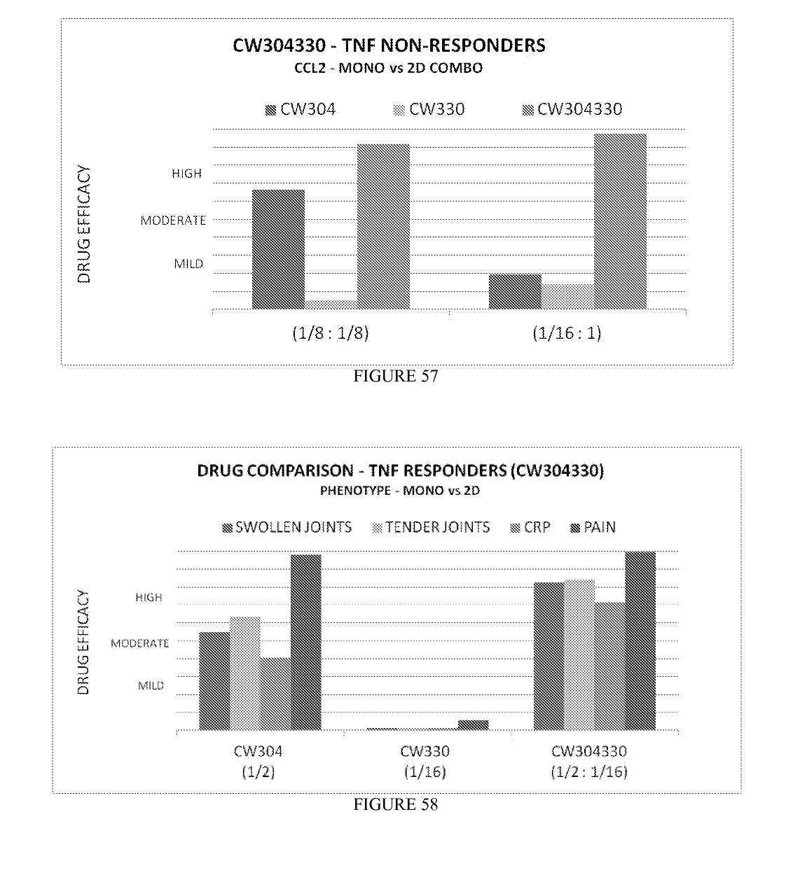

FIGS. 55, 56, and 57 compare the efficacy of the drugs CW304 and CW330 and the combination thereof with regards to TNF, IL6, and CCL2 biomarkers in a TNF-resistive system (anti-TNF non-responders), respectively. The various ratios of the combination of compounds used for the comparison were: a) ⅛:⅛ and (b) 1/16:1.

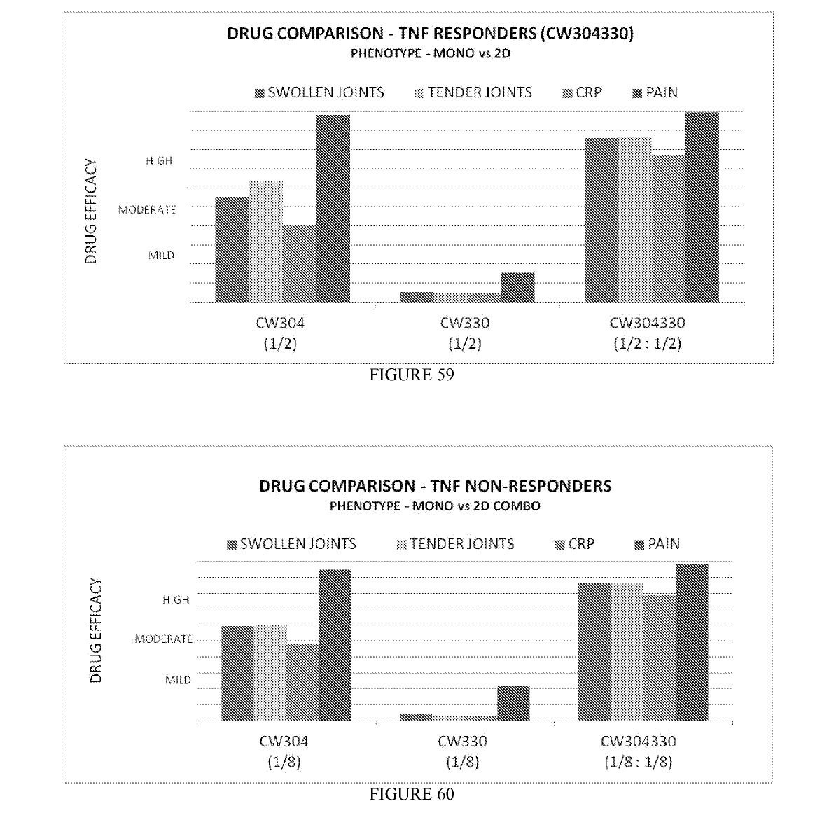

FIGS. 58 and 59 compare the efficacy of the individual drugs CW304 and CW330 with a combination thereof at same dosage. The comparison was done across clinically-measurable parameters including swollen joints, tender joints, CRP, and pain in TNF responders. The various ratios of the combination of compounds used for the comparison were: (a) ½:½ and (b) ½: 1/16, which correspond to FIGS. 58 and 59, respectively.

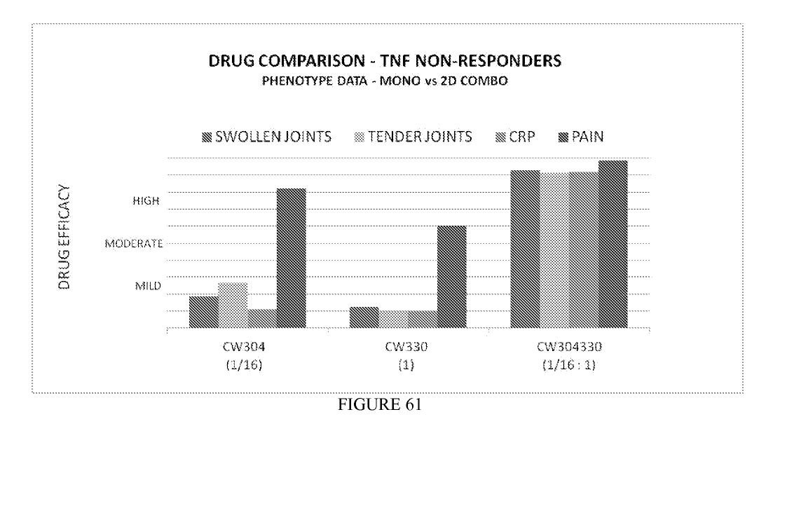

FIGS. 60 and 61 compare the efficacy of the individual drugs CW304 and CW330 with a combination thereof at same dosage. The comparison was done across clinically-measurable parameters including swollen joints, tender joints, CRP, and pain in a TNF-resistant (anti-TNF non-responsive) system. The various ratios of the combination of compounds used for the comparison were: a) ⅛:⅛ and (b) 1/16:1, which correspond to FIGS. 60 and 61, respectively.

### EXAMPLES

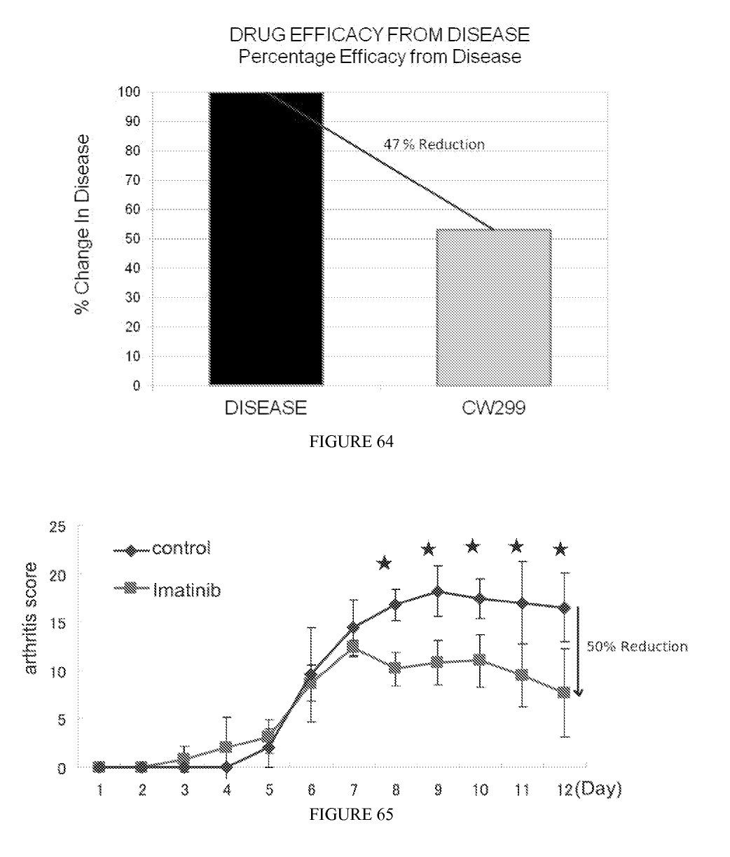

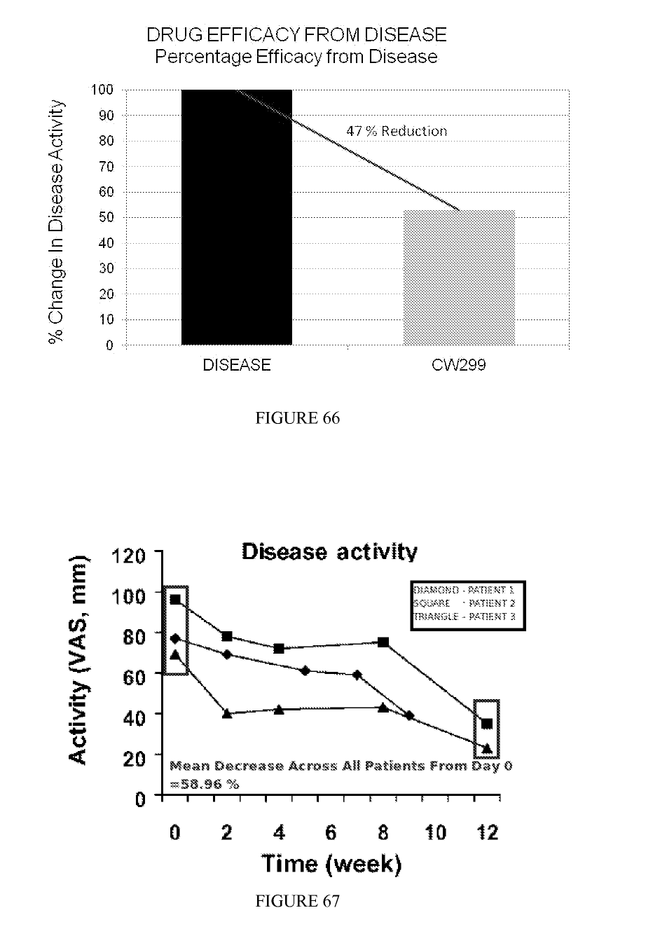

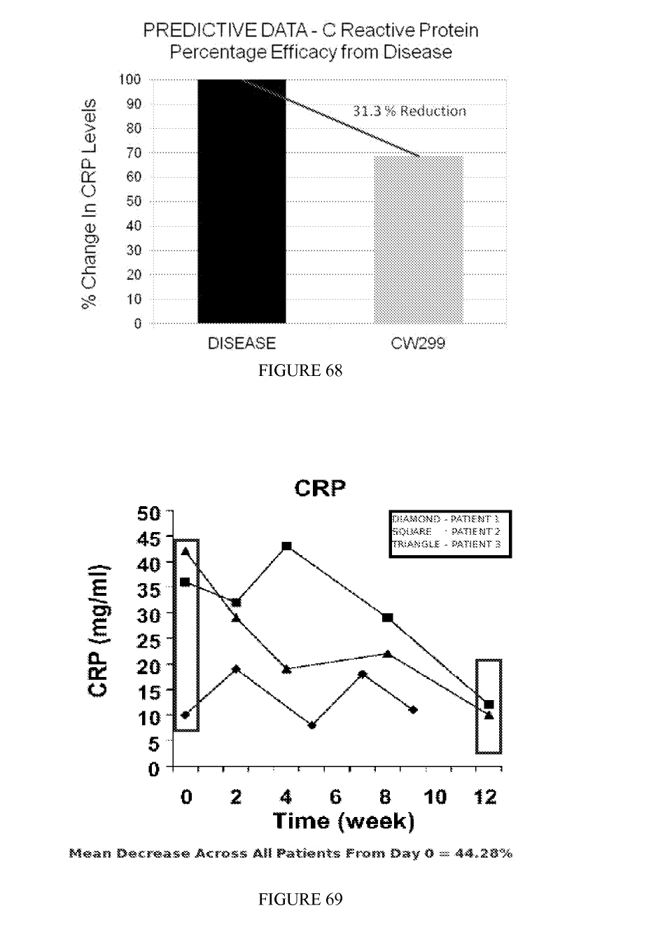

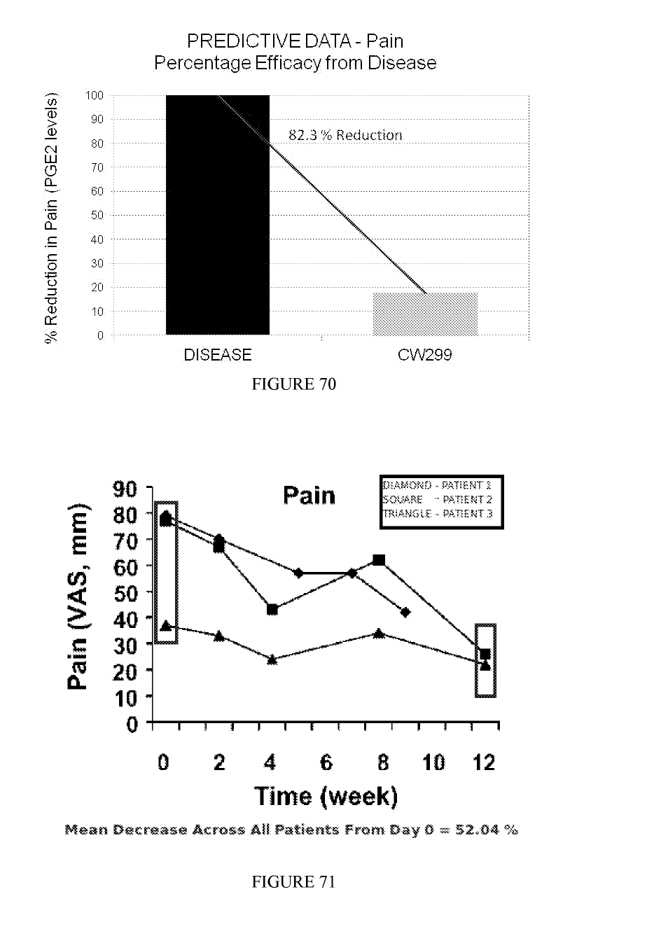

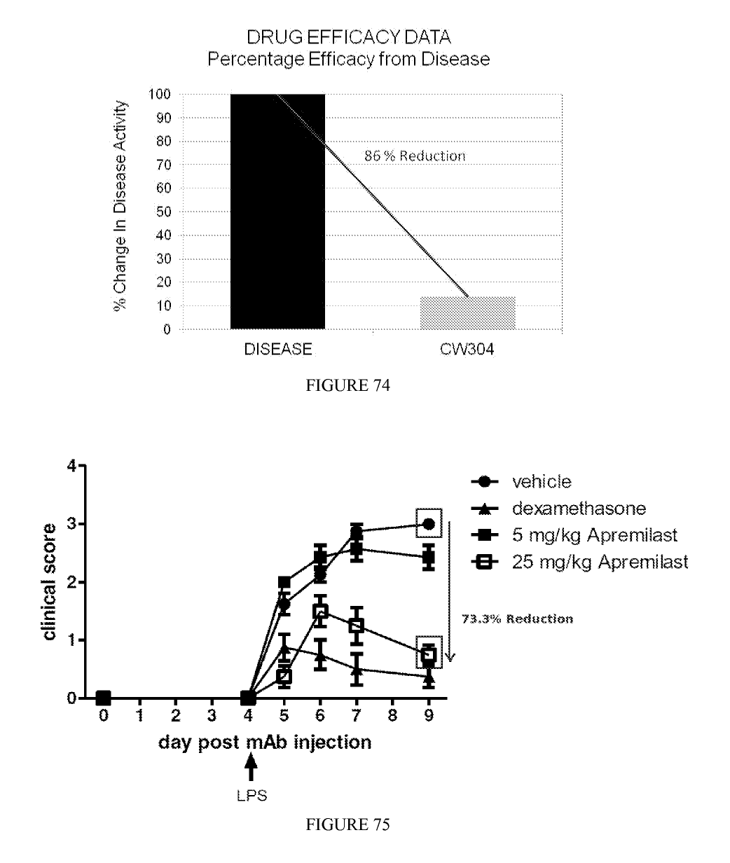

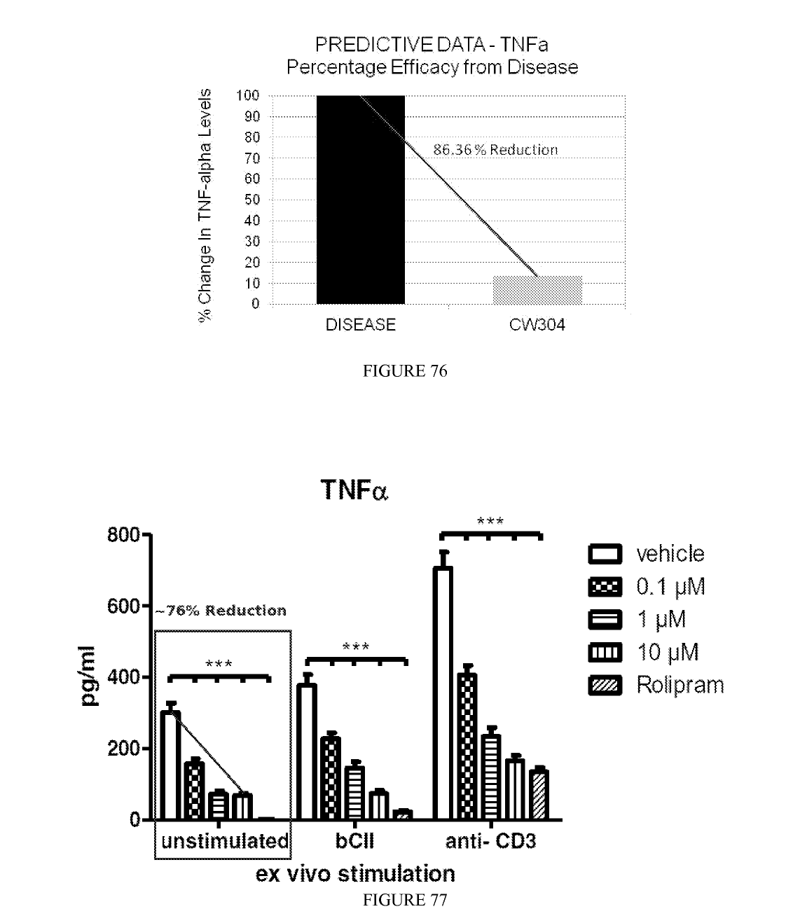

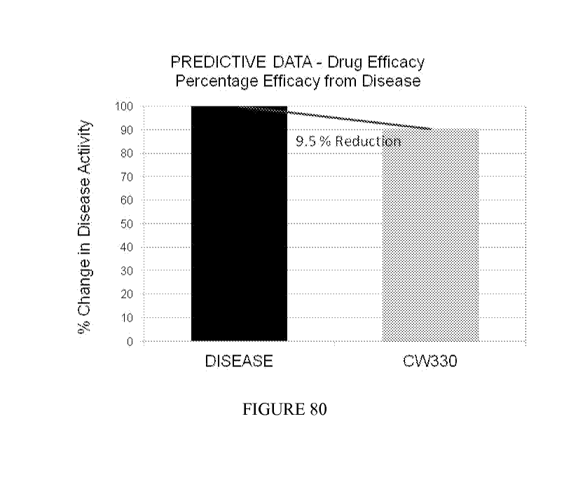

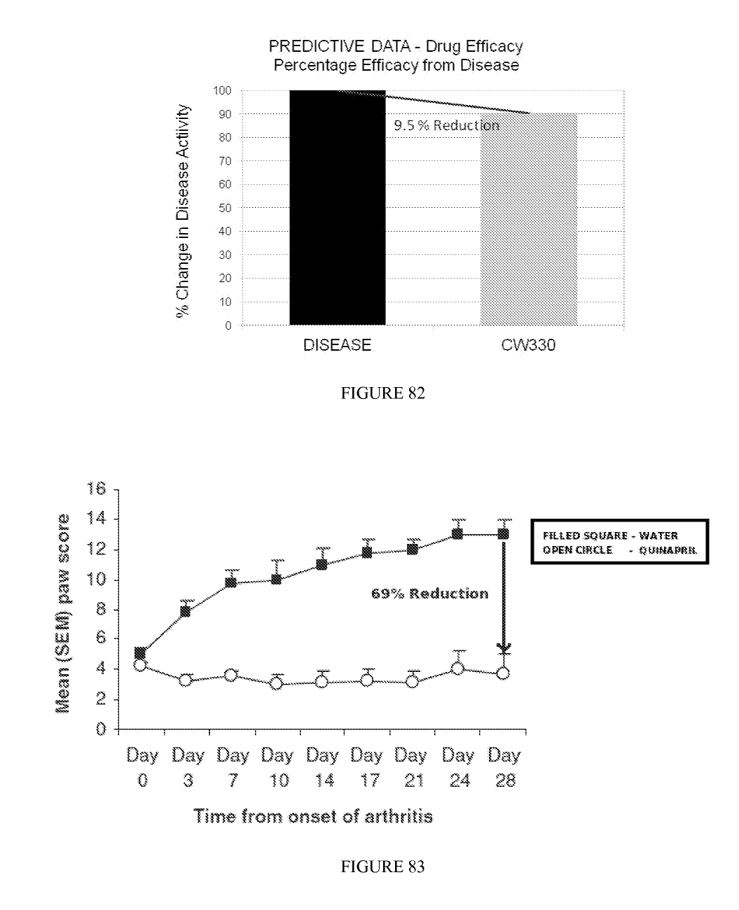

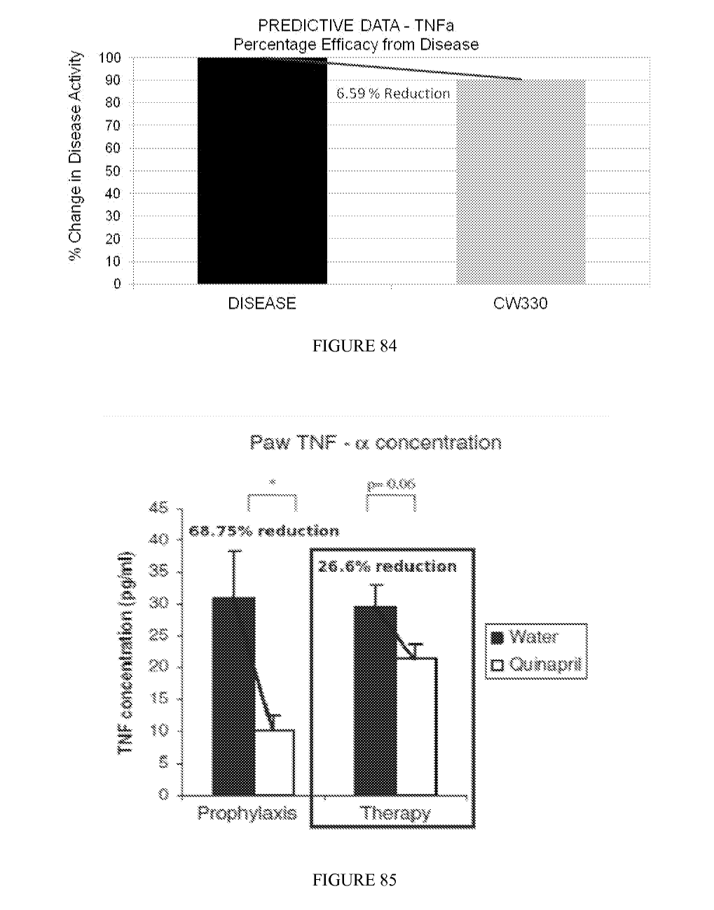

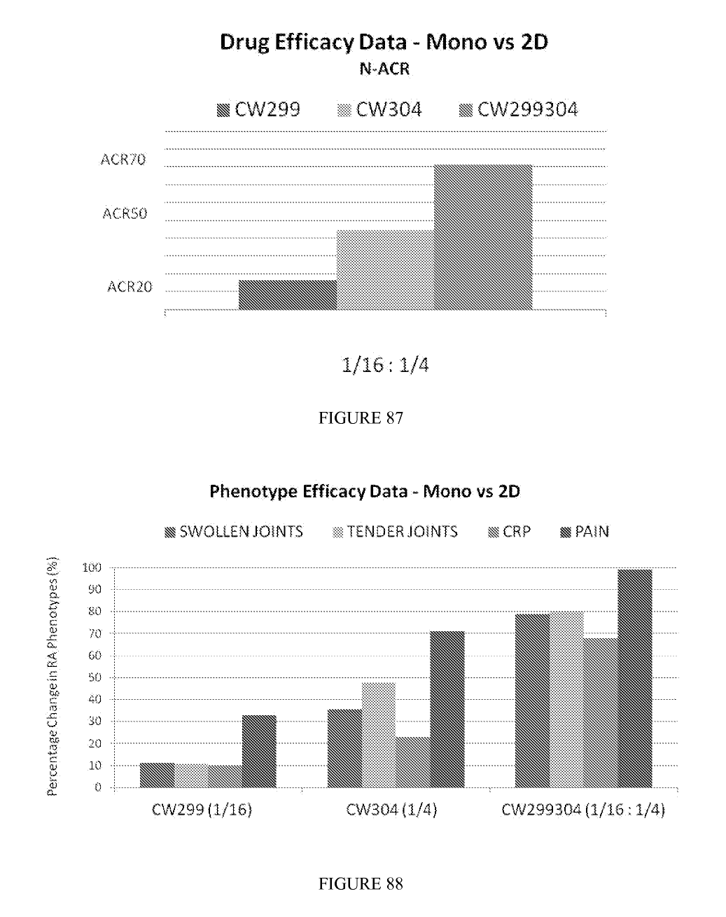

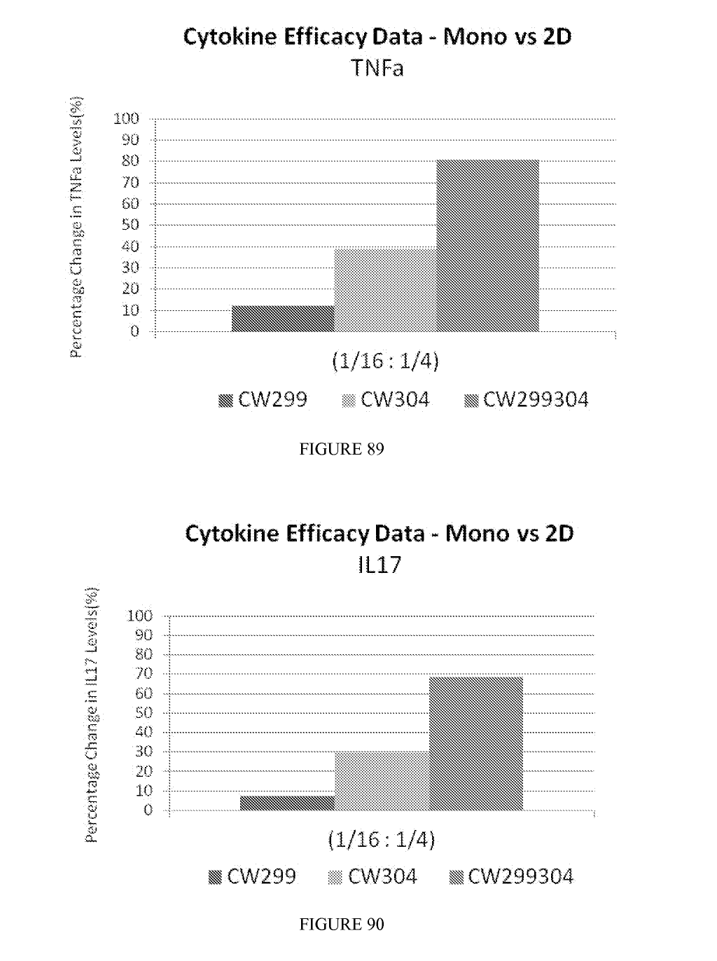

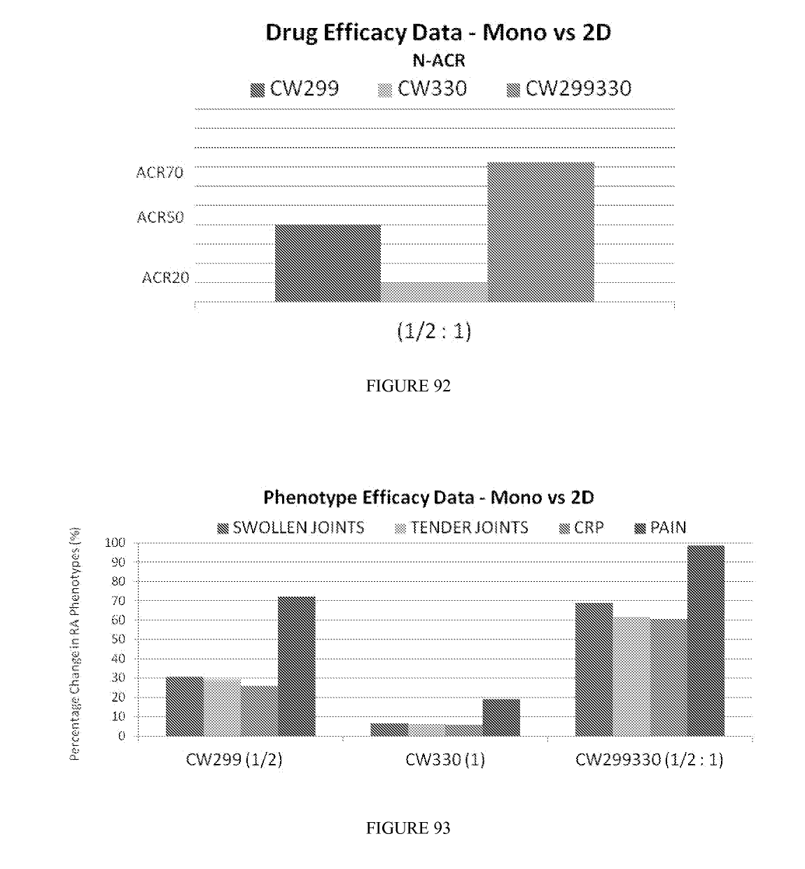

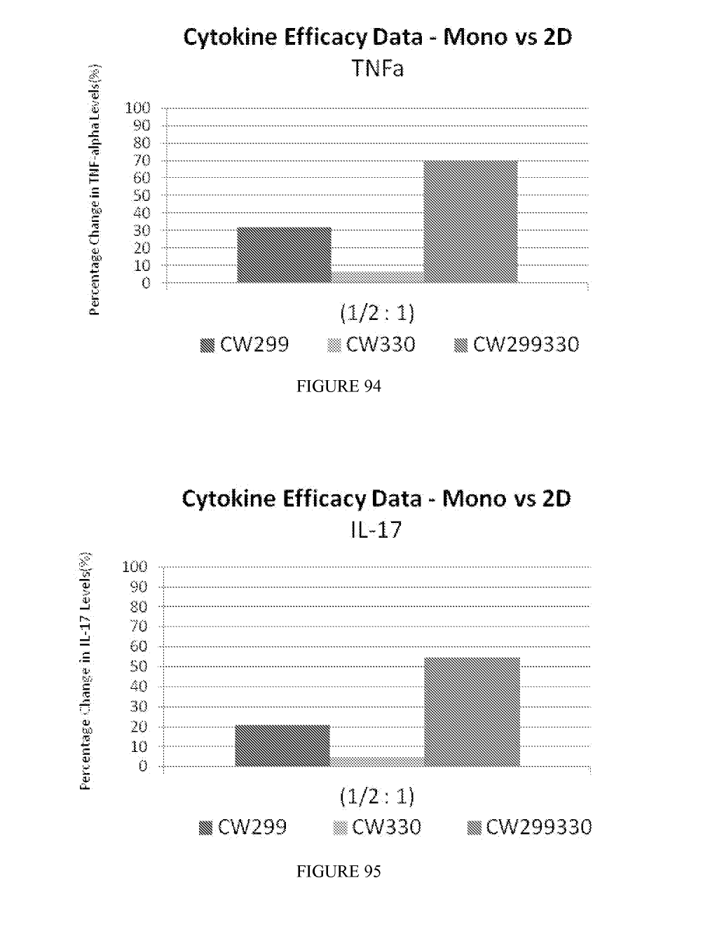

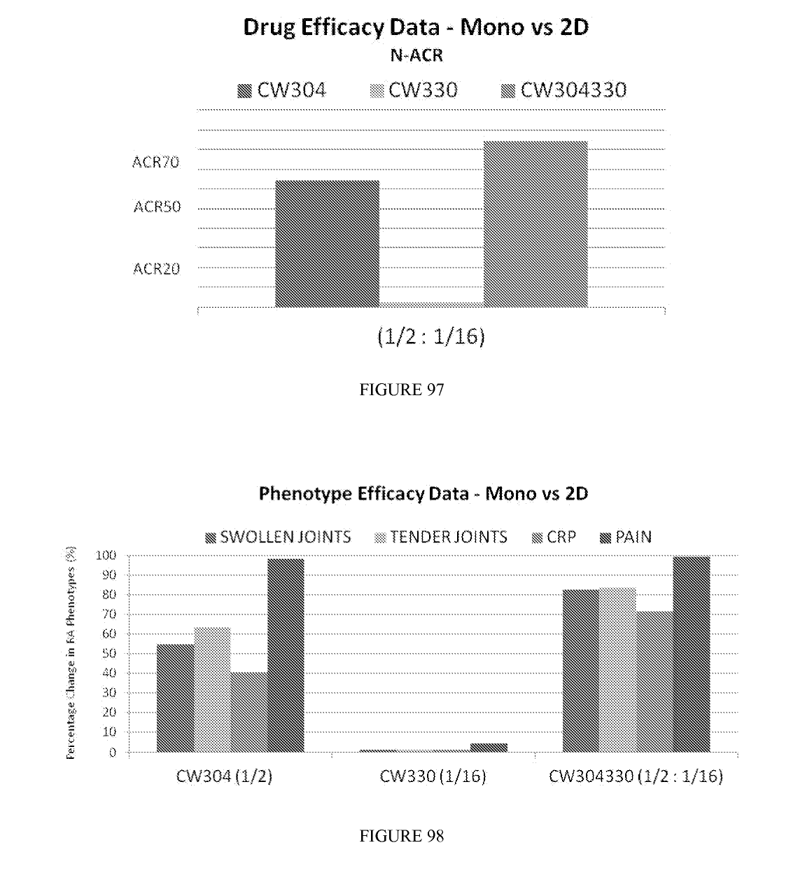

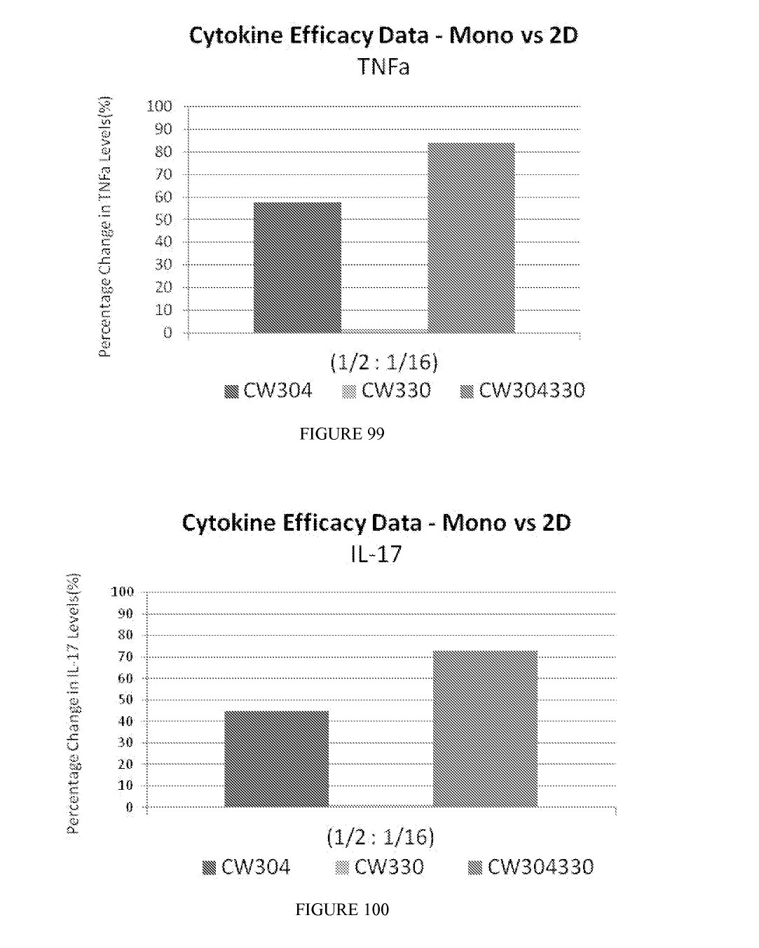

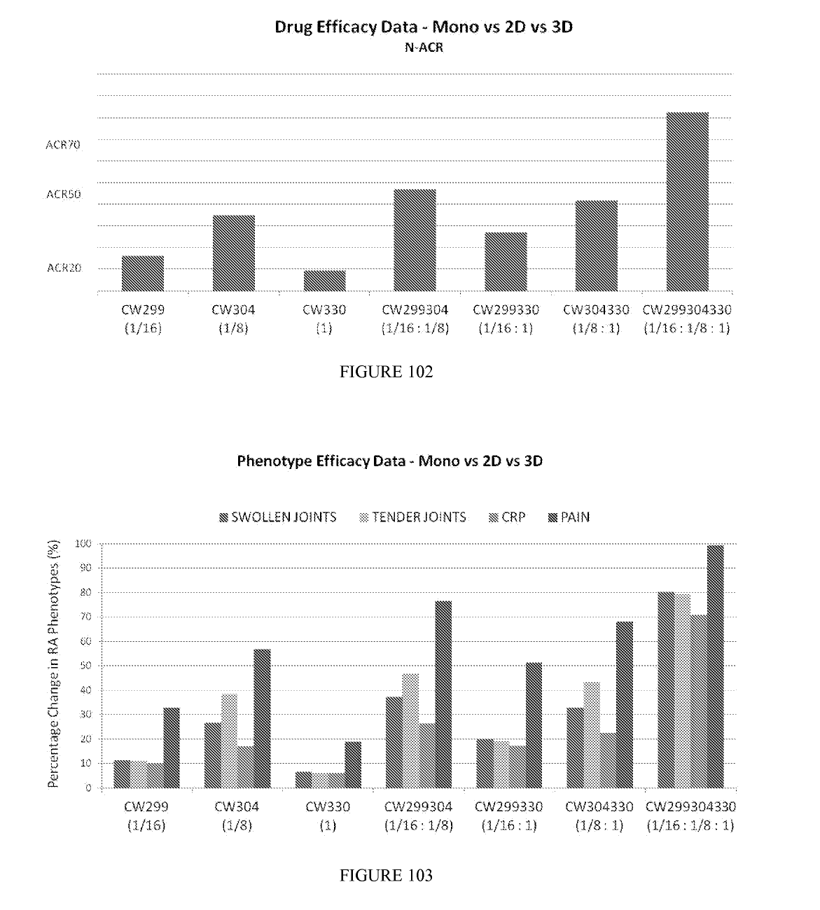

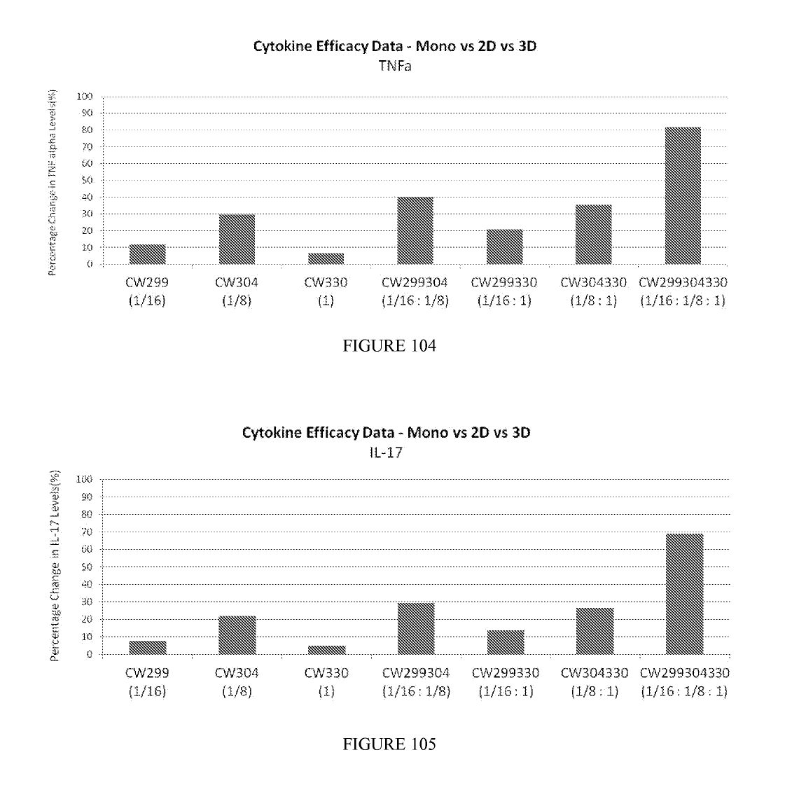

The following examples illustrate various immunological models, and do not limit the scope of the present disclosure.

| Example | Description of the Example | FIGS. |
|---|---|---|
| Example 1 | Efficacy of Imatinib mesylate | FIG. 64 |
| Example 2 | Activity of Imatinib | FIG. 66 |
| Example 3 | Activity of Imatinib on CRP | FIG. 68 |
| Example 4 | Activity of Imatinib on Pain | FIG. 70 |
| Example 5 | Efficacy of Apremilast | FIG. 74 |
| Example 6 | Efficacy of Apremilast on TNFα | FIG. 76 |
| Example 7 | Efficacy of Olmesartan | FIG. 80 |
| Example 8 | Efficacy of Quinapril | FIG. 82 |
| Example 9 | Efficacy of Quinapril on TNFα | FIG. 84 |
| Example 10 | Efficacy of CW299 and CW304 N-ACR | FIG. 87 |
| Example 11 | Efficacy of CW299 and CW304 based on clinical parameters | FIG. 88 |
| | including swollen joints, tender joints, CRP and pain | |
| Example 12 | Efficacy of CW299 and CW304 based on TNFα | FIG. 89 |
| Example 13 | Efficacy of CW299 and CW304 based on IL-17 | FIG. 90 |
| Example 14 | Efficacy of CW299 and CW330 based on N-ACR | FIG. 92 |
| Example 15 | Efficacy of CW299 and CW330 based on clinical parameters | FIG. 93 |
| | including swollen joints, tender joints, CRP and pain | |
| Example 16 | Efficacy of CW299 and CW330 based on TNFα | FIG. 94 |
| Example 17 | Efficacy of CW299 and CW330 based on IL-17 | FIG. 95 |
| Example 18 | Efficacy of CW304 and CW330 based on N-ACR | FIG. 97 |
| Example 19 | Efficacy of CW304 and CW330 based on clinical parameters | FIG. 98 |
| | including swollen joints, tender joints, CRP, and pain | |
| Example 20 | Efficacy of CW304 and CW330 based on TNFα | FIG. 99 |
| Example 21 | Efficacy of CW304 and CW330 based on IL-17 | FIG. 100 |
| Example 22 | Efficacy of CW299, CW304, and CW330 and 2-drug and 3- | FIG. 102 |
| | drug combinations thereof based on N-ACR | |
| Example 23 | Efficacy of CW299, CW304, and CW330 and 2-drug and 3- | FIG. 103 |
| | drug combinations thereof based on clinical parameters | |
| | including swollen joints, tender joints, CRP, and pain | |
| Example 24 | Efficacy of the compounds CW299, CW304, and CW330 and | FIG. 104 |
| | 2-drug and 3-drug combinations thereof based on TNFα | |
| Example 25 | Efficacy of the compounds CW299, CW304, and CW330 and | FIG. 105 |
| | 2-drug and 3-drug combinations thereof based on IL-17 | |

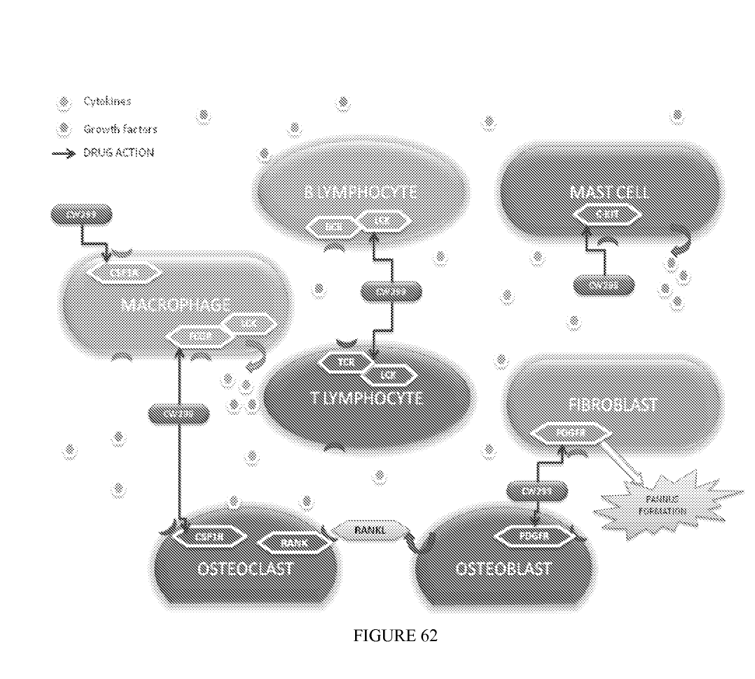

FIG. 62 illustrates examples of pathways associated with the activity of CW299.

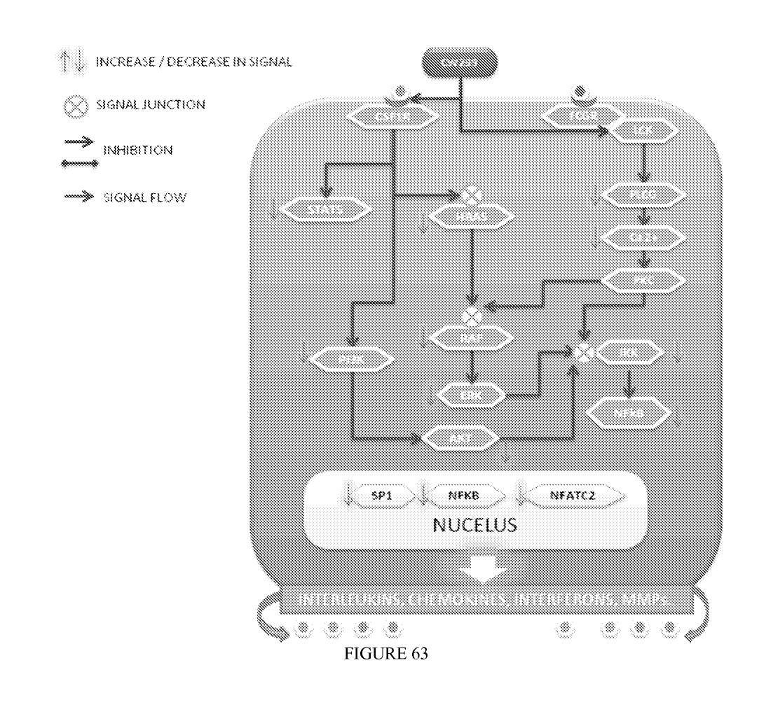

FIG. 63 illustrates biochemical targets associated with CW299.

### Example 1

### Effect of Imatinib Mesylate on Collagen-Induced Arthritis and Comparison with Predictive Data Obtained by Virtual Co-Culture System

FIG. 64 illustrates predictive data for the efficacy of CW299, obtained from a computational experiment using the virtual co-culture described herein. CW299 simulates the biological effects of imatinib mesylate. The figure represents percentage decrease in disease activity with CW299 administration based on data extracted from the virtual co-culture.

A literature example of in vivo efficacy for imatinib mesylate is provided for comparison: Koyama K et. al. Imatinib mesylate both prevents and treats the arthritis induced by type II collagen antibody in mice. Mod Rheumatol. 2007; 17(4):306-10, which is incorporated by reference in its entirety. In Koyama, mice were intraperitoneally injected with 2 mg per mouse of anti-collagen type II mAb cocktail (day 1) and 3 days later (day 4) with 50 μg per mouse of LPS. Imatinib mesylate (1 mg and 10 mg/kg per mouse) or a control vehicle (PBS) was intraperitoneally administered on day 7 and thereafter every day. Clinical scoring was measured during the course of study. Values represent the mean±SD of three mice per group. *P<0.05 compared with corresponding control. Similar results were obtained from at least three independent experiments.

Koyama's experimental data for the efficacy of imatinib mesylate with respect to the arthritis score are illustrated in FIG. 65. A comparison of Koyama's experimental results with the instant simulation result (FIG. 64) supports the reliability of predictions made using CW299 co-culture simulations as described herein.

### Example 2

### Effect of Imatinib on Rheumatoid Arthritis and Comparison with Predictive Data Obtained by Virtual Co-Culture System

FIG. 66 illustrates predictive data for the efficacy of CW299, obtained from a computational experiment using the virtual co-culture described herein. CW299 simulates the biological effects of imatinib mesylate. The figure represents percentage decrease in disease activity with CW299 administration based on data extracted from the virtual co-culture.

A literature example of in vivo efficacy for imatinib is provided for comparison: Eklund K K et. al. Treatment of rheumatoid arthritis with imatinib mesylate: clinical improvement in three refractory cases. Ann Med. 2003; 35(5):362-7, which is incorporated by reference in its entirety. In Eklund, three patients with severe rheumatoid arthritis were treated with imatinib for 12 weeks. The number of tender and swollen joints, patient-assessed disease activity and pain as assessed by a visual analogue scale, a health assessment questionnaire (HAQ) score; serum C-reactive protein (CRP) and blood erythrocyte sedimentation rate (ESR) were used as the primary outcome measures. Eklund's experimental data for the activity of imatinib with respect to the activity calculated by the visual analogue scale are illustrated in FIG. 67. A comparison of Eklund's experimental results with the instant simulation result (FIG. 66) supports the reliability of predictions made using CW299 co-culture simulations as described herein.

### Example 3

### Effect of Imatinib on Rheumatoid Arthritis and Comparison with Predictive Data Obtained by Virtual Co-Culture System

FIG. 68 illustrates predictive data for the efficacy of CW299, obtained from a computational experiment using the virtual co-culture described herein. CW299 simulates the biological effects of imatinib mesylate. The figure represents percentage decrease in CRP levels with CW299 administration based on data extracted from the virtual co-culture.

A literature example of in vivo efficacy for imatinib is provided for comparison in Eklund, supra. In Eklund, three patients with severe rheumatoid were treated with imatinib for 12 weeks. The number of tender and swollen joints, patient-assessed disease activity and pain as assessed by a visual analogue scale, a health assessment questionnaire (HAQ) score; serum C-reactive protein (CRP) and blood erythrocyte sedimentation rate (ESR) were used as the primary outcome measures. Eklund's experimental data for the activity of imatinib with respect to CRP are illustrated in FIG. 69. A comparison of Eklund's experimental results with the instant simulation result (FIG. 68) supports the reliability of predictions made using CW299 co-culture simulations as described herein.

### Example 4

### Effect of Imatinib on Rheumatoid Arthritis and Comparison with Predictive Data Obtained by Virtual Co-Culture System

FIG. 70 illustrates predictive data for the efficacy of CW299, obtained from a computational experiment using the virtual co-culture described herein. CW299 simulates the biological effects of imatinib mesylate. The figure represents percentage decrease in pain with CW299 administration based on data extracted from the virtual co-culture.

A literature example of in vivo efficacy for imatinib is provided for comparison in Eklund, supra. In Eklund, three patients with severe rheumatoid were treated with imatinib for 12 weeks. The number of tender and swollen joints, patient-assessed disease activity and pain as assessed by a visual analogue scale, a health assessment questionnaire (HAQ) score; serum C-reactive protein (CRP) and blood erythrocyte sedimentation rate (ESR) were used as the primary outcome measures. Eklund's experimental data for the activity of imatinib with respect to pain are illustrated in FIG. 71. A comparison of Eklund's experimental results with the instant simulation result (FIG. 70) supports the reliability of predictions made using CW299 co-culture simulations as described herein.

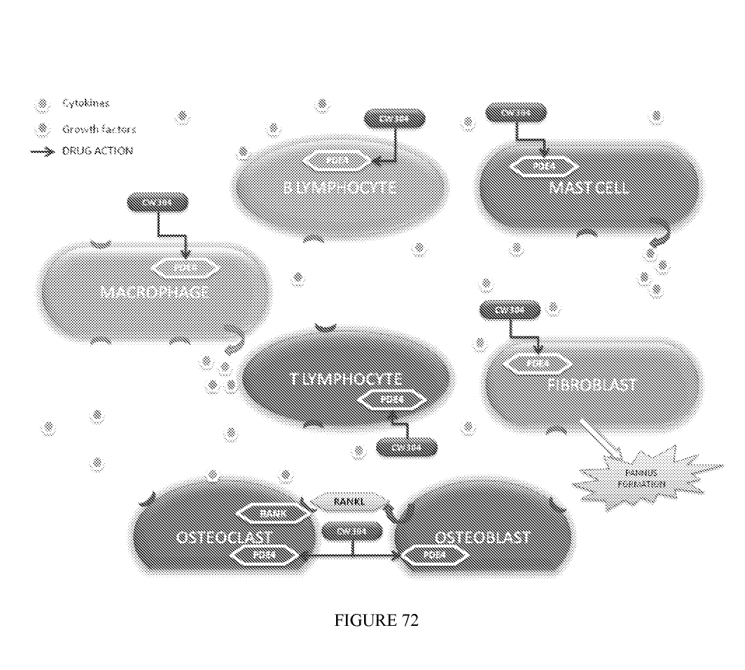

FIG. 72 illustrates examples of pathways associated with the activity of CW304.

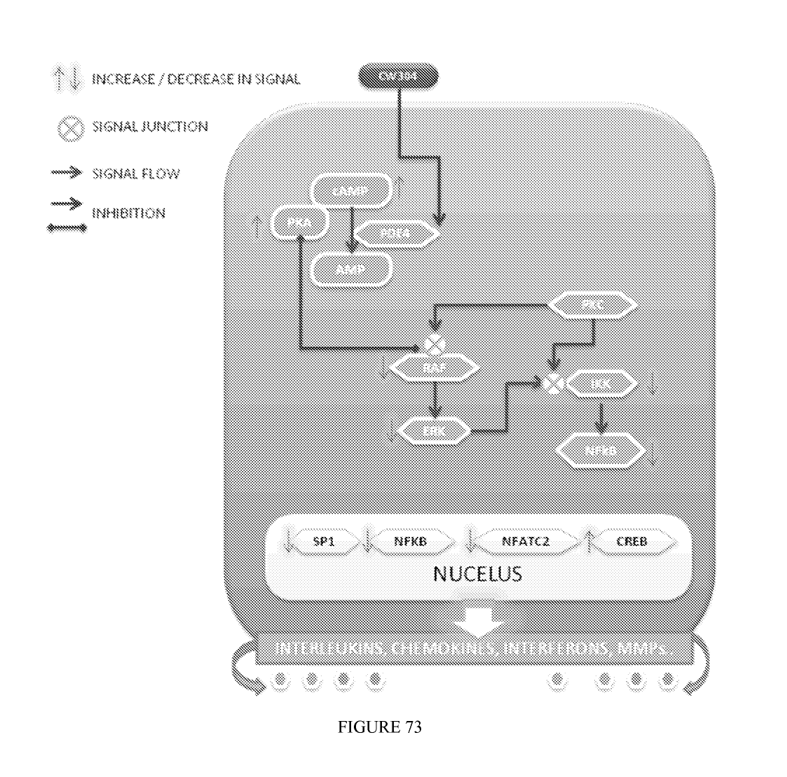

FIG. 73 illustrates biochemical targets associated with CW304.

### Example 5

### Effect of Apremilast on Rheumatoid Arthritis and Comparison with Predictive Data Obtained by Virtual Co-Culture System

FIG. 74 illustrates predictive data for the efficacy of CW304, obtained from a computational experiment using the virtual co-culture described herein. CW304 simulates the biological effects of apremilast. The figure represents percentage decrease in disease with CW304 administration based on data extracted from the virtual co-culture.

A literature example of in vivo efficacy for apremilast is provided for comparison: McCann F E et. al. Apremilast, a novel PDE4 inhibitor, inhibits spontaneous production of tumour necrosis factor-alpha from human rheumatoid synovial cells and ameliorates experimental arthritis. Arthritis Res Ther. 2010; 12(3):R107, which is incorporated by reference in its entirety. In McCann, arthritis was induced in six-week-old male BALB/c mice by intravenous administration of a cocktail of four anticollagen antibodies, followed by LPS (i.p) three days later. At this time, mice (eight per treatment) were given a daily, oral dose of vehicle or dexamethasone, to form negative and positive control groups, respectively, while experimental groups were treated orally with 1, 5 or 25 mg/kg apremilast. Treatment continued for four successive days, until day 7, with close monitoring of disease severity throughout until day 9. Two days after LPS administration (day 5 post injection of mAbs), all mice began to show varying degrees of arthritis severity. Apremilast at 5 and 25 mg/kg and dexamethasone at 1 mg/kg, significantly suppressed arthritis severity, as measured by clinical score. McCann's experimental data for the efficacy of apremilast with respect to the clinical score are illustrated in FIG. 75. A comparison of McCann's experimental results with the instant simulation result (FIG. 74) supports the reliability of predictions made using CW304 co-culture simulations as described herein.

### Example 6

### Effect of Apremilast on Rheumatoid Arthritis and Comparison with Predictive Data Obtained by Virtual Co-Culture System

FIG. 76 illustrates predictive data for the efficacy of CW304 for TNFα, obtained from a computational experiment using the virtual co-culture described herein. CW304 simulates the biological effects of apremilast. The figure represents percentage decrease in levels of TNFα with CW304 administration based on data extracted from the virtual co-culture.

A literature example of in vivo efficacy for apremilast is provided for comparison in McCann, supra. In McCann, lymph node cultures from male DBA/1 mice immunised with bCII in CFA were unstimulated, or stimulated with bCII or anti-CD3 mAb, in the presence of increasing concentrations of apremilast or 100 μM rolipram, (used as positive control only), for three days. After 48 hours, supernatants were collected for cytokine analysis. McCann's experimental data for the efficacy of apremilast with respect to TNFα levels are illustrated in FIG. 77. A comparison of McCann's experimental results with the instant simulation result (FIG. 76) supports the reliability of predictions made using CW304 co-culture simulations as described herein.

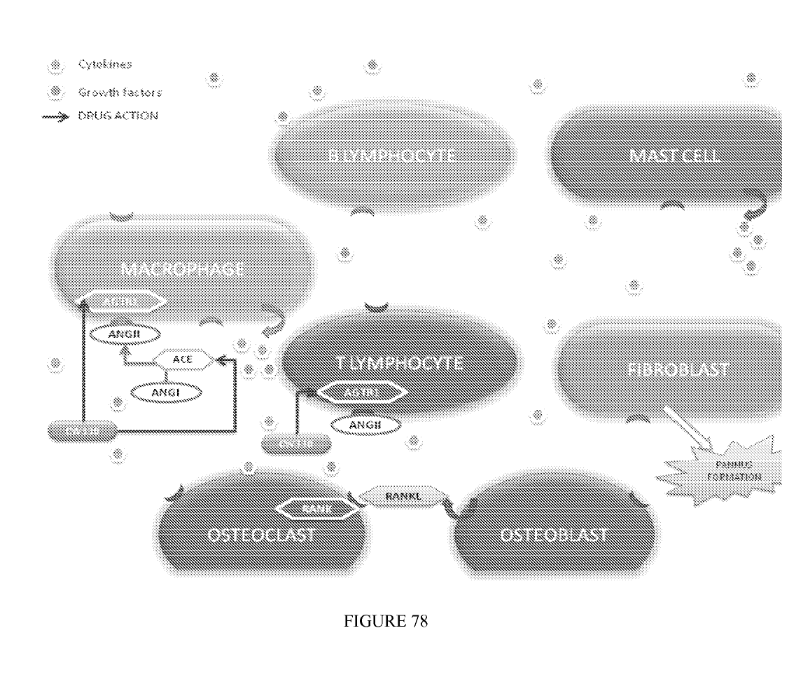

FIG. 78 illustrates examples of pathways associated with the activity of CW330.

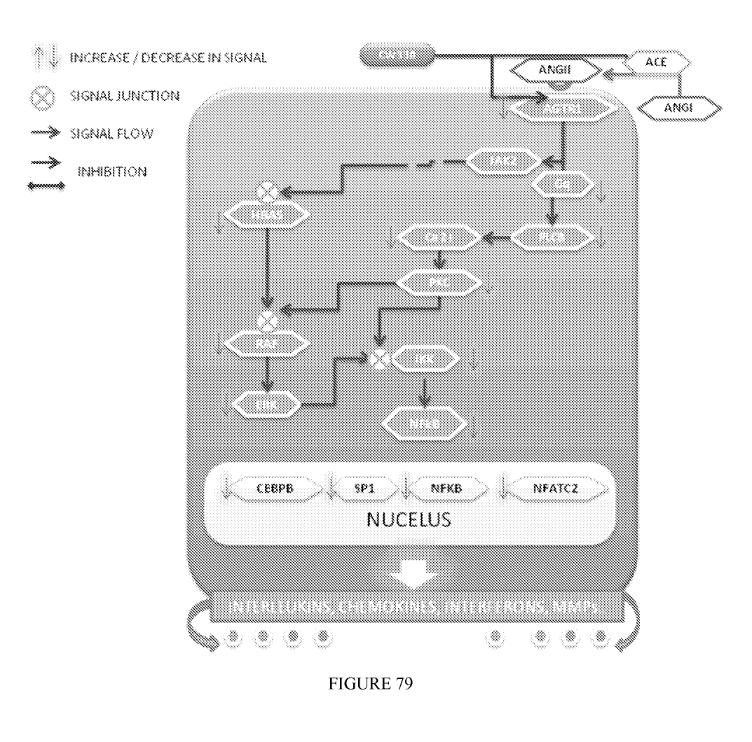

FIG. 79 illustrates biochemical targets associated with CW330.

### Example 7

### Effect of Olmesartan on Rheumatoid Arthritis and Comparison with Predictive Data Obtained by Virtual Co-Culture System

FIG. 80 illustrates predictive data for the efficacy of CW330 for TNFα, obtained from a computational experiment using the virtual co-culture described herein.

CW330 simulates the biological effects of olmesartan. The figure represents percentage decrease in disease with CW330 administration based on data extracted from the virtual co-culture.

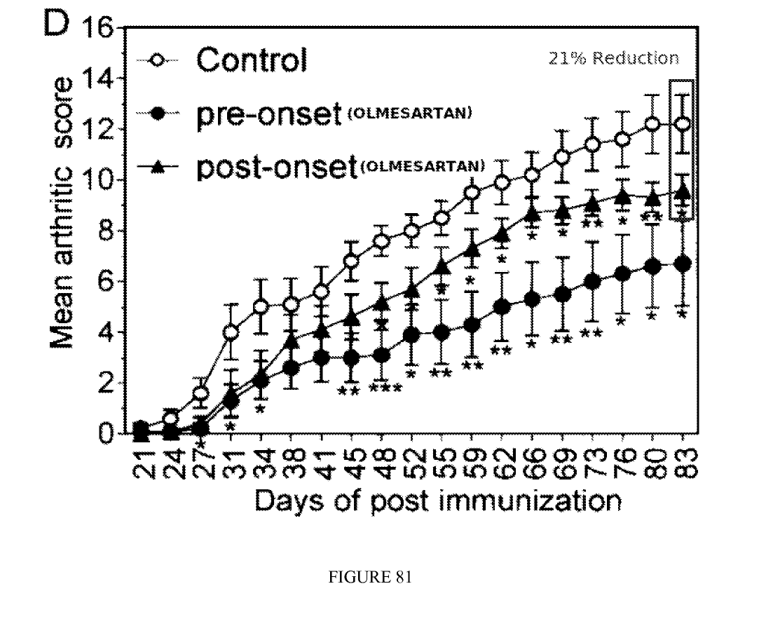

A literature example of in vivo efficacy for olmesartan is provided for comparison: Sagawa K et. al. Angiotensin receptor blockers suppress antigen-specific T cell responses and ameliorate collagen-induced arthritis in mice. Arthritis Rheum. 2005 June; 52(6):1920-8, which is incorporated by reference herein in its entirety. In Sagawa, arthritis was induced in DBA/1 mice by immunization with type II collagen (CII) in Freund's complete adjuvant on day 0. On day 21, mice were injected subcutaneously with CII in Freund's incomplete adjuvant. Olmesartan (10 mg/kg) or vehicle only was administered, beginning on day 25 and continued until day 87. Sagawa's experimental data for the efficacy of the compound CW330 in terms of mean arthritic score are illustrated in FIG. 81. A comparison of Sagawa's experimental results with the instant simulation result (FIG. 80) supports the reliability of predictions made using CW330 co-culture simulations as described herein.

### Example 8

### Effect of Ouinapril on Rheumatoid Arthritis and Comparison with Predictive Data Obtained by Virtual Co-Culture System

FIG. 82 illustrates predictive data for the efficacy of CW330, obtained from a computational experiment using the virtual co-culture described herein. CW330 simulates the biological effects of quinapril. The figure represents percentage decrease in disease with CW330 administration based on data extracted from the virtual co-culture.

A literature example of in vivo efficacy for quinapril is provided for comparison: Dalbeth N et. al. The non-thiol angiotensin-converting enzyme inhibitor quinapril suppresses inflammatory arthritis. Rheumatology (Oxford). 2005 January; 44(1):24-31, which is incorporated by reference in its entirety. In Dalbeth, arthritis was induced in DBA/1 mice by immunization with bCII/CFA. Mice were randomized to receive water (filled squares, n=7) or quinapril (open circles, n=7) when the total paw score reached 4. Day 0 denotes the day of randomization. Quinapril reduces the severity of collagen-induced arthritis. Dalbeth's experimental data for the efficacy of quinapril with respect to mean (SEM) paw score is illustrated in FIG. 83. A comparison of Dalbeth's experimental results with the instant simulation result (FIG. 82) supports the reliability of predictions made using CW330 co-culture simulations as described herein.

### Example 9

### Effect of Ouinapril on Rheumatoid Arthritis and Comparison with Predictive Data Obtained by Virtual Co-Culture System

FIG. 84 illustrates predictive data for the activity of the compound CW330 for TNFα, obtained from a computational experiment using the virtual co-culture described herein. CW330 simulates the biological effects of quinapril. The figure represents percentage decrease in disease with CW330 administration based on data extracted from the virtual co-culture.

A literature example of in vivo efficacy for quinapril is provided for comparison in Dalbeth, supra. In Dalbeth, arthritis was induced in DBA/1 mice by immunization with bCII/CFA. Mice were randomized to receive water or quinapril from the time of immunization (n=23) or when the total paw score reached 4 (n=14). TNF-α levels were measured on forepaw homogenates by ELISA. Quinapril reduced the severity of collagen-induced arthritis. Dalbeth's experimental data for the efficacy of the compound CW330 with respect to levels of TNFα are illustrated in FIG. 85. A comparison of Dalbeth's experimental results with the instant simulation result (FIG. 84) supports the reliability of predictions made using CW330 co-culture simulations as described herein.

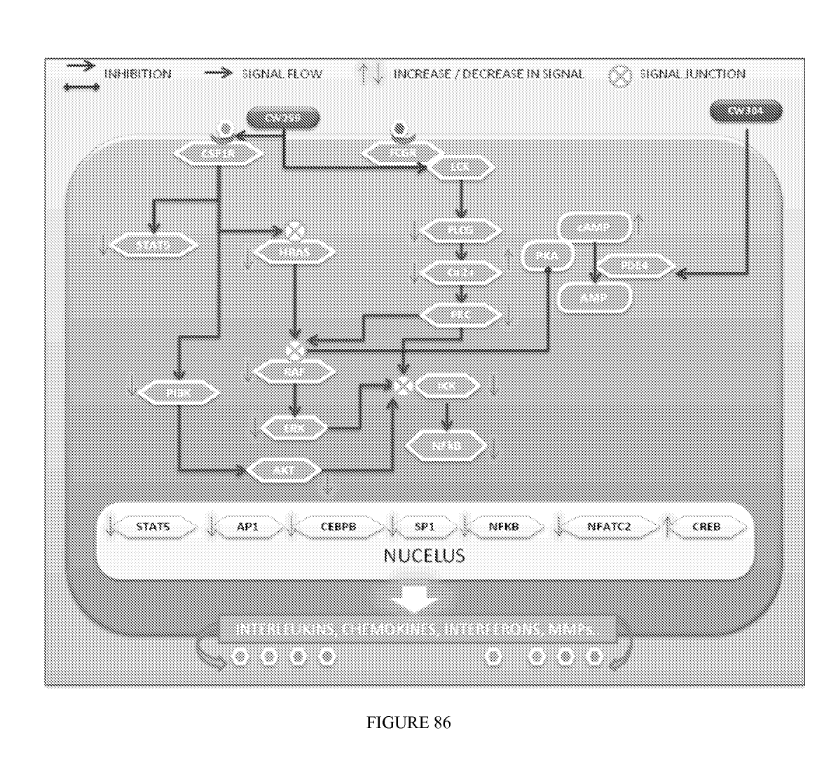

FIG. 86 illustrates examples of pathways associated with the activity of CW299 and CW304.

### Example 10

### Synergistic Effect of CW299 and CW304 on Rheumatoid Arthritis

Data extracted from a virtual co-culture, simulated as described herein, for the efficacy of the individual drugs CW299, CW304 and the combination thereof based on N-ACR score, are illustrated in FIG. 87 and in the table below.

| | | | | | % Synergy |
|---|---|---|---|---|---|
| | % Efficacy | | | | CW299304 − |
| | | | CW299 + | | (CW299 + |
| Marker | CW299 | CW304 | CW304 | CW299304 | CW304) |
| N-ACR | 16.26 | 44.34 | 60.61 | 81.57 | 20.96 |

### Example 11

### Synergistic Effect of CW299 and CW304 on Rheumatoid Arthritis

Data extracted from a virtual co-culture, simulated as described herein, for the efficacy of the individual drugs CW299, CW304 and the combination thereof based on different clinical parameters such as swollen joints, tender joints, CRP, and pain, are illustrated in FIG. 88 and in the table below.

| | | | | | % Synergy |
|---|---|---|---|---|---|
| | % Efficacy | | | | CW299304 − |
| | | | CW299 + | | (CW299 + |
| Phenotype | CW299 | CW304 | CW304 | CW299304 | CW304) |
| SWOL- | 11.28 | 35.58 | 46.86 | 79.09 | 32.22 |
| LEN | | | | | |
| JOINTS | | | | | |
| TENDER | 11.02 | 47.7 | 58.72 | 80.01 | 21.29 |
| JOINTS | | | | | |
| CRP | 9.91 | 22.99 | 32.9 | 68 | 35.1 |
| PAIN | 32.85 | 71.12 | 103.97 | 99.2 | −4.77 |

### Example 12

### Synergistic Effect of CW299 and CW304 on Rheumatoid Arthritis

Data extracted from a virtual co-culture, simulated as described herein, for the efficacy of the individual drugs CW299, CW304 and the combination thereof based on TNFα are illustrated in FIG. 89 and in the table below.

| | | | | | % Synergy |
|---|---|---|---|---|---|
| | % Efficacy | | | | CW299304 − |
| | | | CW299 + | | (CW299 + |
| Marker | CW299 | CW304 | CW304 | CW299304 | CW304) |
| TNFα | 11.9 | 38.76 | 50.66 | 80.76 | 30.1 |

### Example 13

### Synergistic Effect of CW299 and CW304 on Rheumatoid Arthritis

Data extracted from a virtual co-culture, simulated as described herein, for the efficacy of the individual drugs CW299, CW304 and the combination thereof based on IL-17 is illustrated in FIG. 90 and in the table below.

| | | | | | % Synergy |
|---|---|---|---|---|---|
| | % Efficacy | | | | CW299304 − |
| | | | CW299 + | | (CW299 + |
| Marker | CW299 | CW304 | CW304 | CW299304 | CW304) |
| IL-17 | 7.6 | 29.45 | 37.05 | 68.42 | 31.37 |

The data provided in examples 15 to 18, and correspondingly in FIGS. 87 to 90, indicate that the combination of CW299 and CW304 provides for a considerable synergistic effect, when compared with the individual drugs and their theoretical additive efficacy. This synergism was observed with respect to various clinical phenotypic parameters such as swollen joints, tender joints, and CRP, as well as at biomarker level, when cytokines such as TNFα and IL-17 were assayed.

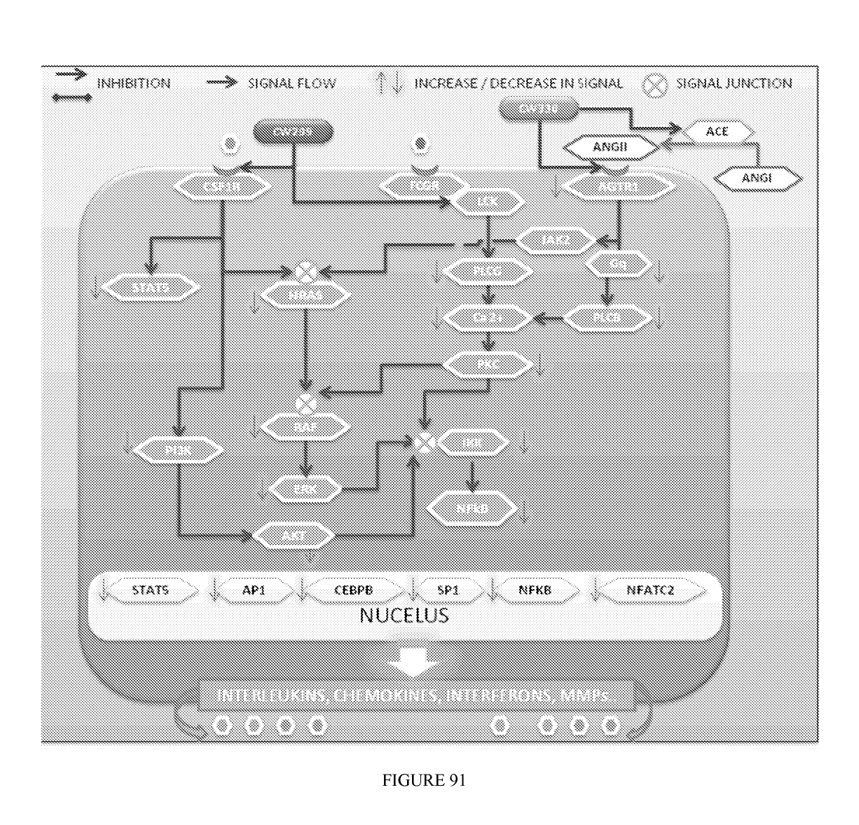

FIG. 91 illustrates examples of pathways associated with the activity of CW299 and CW330.

### Example 14

### Synergistic Effect of CW299 and CW330 on Rheumatoid Arthritis

Data extracted from a virtual co-culture, simulated as described herein, for the efficacy of the individual drugs CW299, CW330 and the combination thereof based on N-ACR score are illustrated in FIG. 92 and in the table below.

| | | | | | % Synergy |
|---|---|---|---|---|---|
| | % Efficacy | | | | CW299330 − |
| | | | CW299 + | | (CW299 + |
| Marker | CW299 | CW330 | CW330 | CW299330 | CW330) |
| N-ACR | 39.44 | 9.49 | 48.93 | 72.43 | 23.5 |

### Example 15

### Synergistic Effect of CW299 and CW330 on Rheumatoid Arthritis

Data extracted from a virtual co-culture, simulated as described herein, for the efficacy of the individual drugs CW299, CW330 and the combination thereof based on different clinical parameters such as swollen joints, tender joints, CRP, and pain are illustrated in FIG. 93 and in the table below.

| | | | | | % Synergy |
|---|---|---|---|---|---|
| | % Efficacy | | | | CW299330 − |
| | | | CW299 + | | (CW299 + |
| Phenotype | CW299 | CW330 | CW330 | CW299330 | CW330) |
| SWOL- | 30.75 | 6.64 | 37.39 | 68.79 | 31.4 |
| LEN | | | | | |
| JOINTS | | | | | |
| TENDER | 29.17 | 6.33 | 35.5 | 61.69 | 26.19 |
| JOINTS | | | | | |
| CRP | 25.86 | 5.97 | 31.83 | 60.45 | 28.62 |
| PAIN | 72.02 | 19 | 91.02 | 98.81 | 7.79 |

### Example 16

### Synergistic Effect of CW299 and CW330 on Rheumatoid Arthritis

Data extracted from a virtual co-culture, simulated as described herein, for the efficacy of the individual drugs CW299, CW330 and the combination thereof based on TNFα are illustrated in FIG. 94 and in the table below.

| | | | | | % Synergy |
|---|---|---|---|---|---|
| | % Efficacy | | | | CW299330 − |
| | | | CW299 + | | (CW299 + |
| Marker | CW299 | CW330 | CW330 | CW299330 | CW330) |
| TNFα | 32.04 | 6.59 | 38.63 | 69.99 | 31.36 |

### Example 17

### Synergistic Effect of CW299 and CW304 on Rheumatoid Arthritis

Data extracted from a virtual co-culture, simulated as described herein, for the efficacy of the individual drugs CW299, CW330 and the combination thereof based on IL-17 are illustrated in FIG. 95 and in the table below.

| | | | | | % Synergy |
|---|---|---|---|---|---|
| | % Efficacy | | | | CW299330 − |
| | | | CW299 + | | (CW299 + |
| Marker | CW299 | CW330 | CW330 | CW299330 | CW330) |
| IL-17 | 21.04 | 4.78 | 25.82 | 54.43 | 28.61 |

The data provided in examples 19 to 22, and correspondingly in FIGS. 92 to 95, indicate that the combination of CW299 and CW330 provides for a considerable synergistic effect, when compared with the individual drugs and their theoretical additive efficacy. This synergism was observed with respect to various clinical phenotypic parameters such as swollen joints, tender joints, CRP, and pain, as well as at biomarker level, when cytokines such as TNFα and IL-17 were assayed.

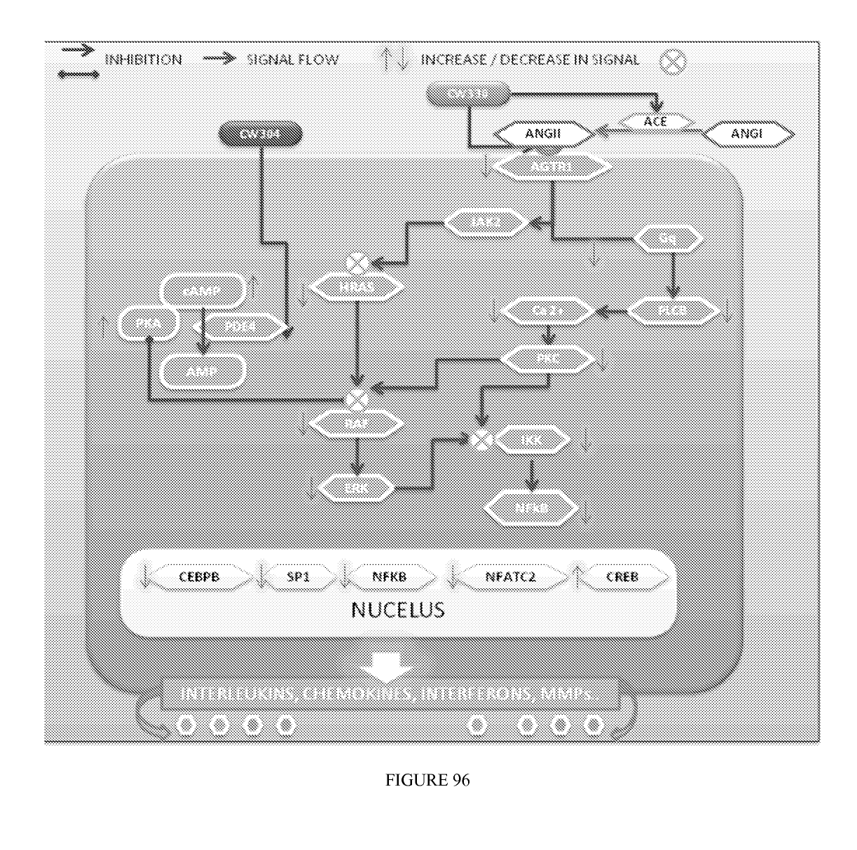

FIG. 96 illustrates examples of pathways associated with the activity of CW304 and CW330.

### Example 18

### Synergistic Effect of CW304 and CW330 on Rheumatoid Arthritis

Data extracted from a virtual co-culture, simulated as described herein, for the efficacy of the individual drugs CW304, CW330 and the combination thereof based on N-ACR score are illustrated in FIG. 97 and in the table below.

| | | | | | % Synergy |
|---|---|---|---|---|---|
| | % Efficacy | | | | CW304330 − |
| | | | CW304 + | | (CW304 + |
| Marker | CW304 | CW330 | CW330 | CW304330 | CW330) |
| N-ACR | 64.2 | 2.19 | 66.39 | 84.24 | 17.84 |

### Example 19

### Synergistic Effect of CW304 and CW330 on Rheumatoid Arthritis

Data extracted from a virtual co-culture, simulated as described herein, for the efficacy of the individual drugs CW304, CW330 and the combination thereof based on different clinical parameters such as swollen joints, tender joints, CRP, and pain is illustrated in FIG. 98 and in the table below.

| | | | | | % Synergy |
|---|---|---|---|---|---|
| | % Efficacy | | | | CW304330 − |
| | | | CW304 + | | (CW304 + |
| Phenotype | CW304 | CW330 | CW330 | CW304330 | CW330) |
| SWOL- | 54.89 | 1.48 | 56.36 | 82.45 | 26 |
| LEN | | | | | |
| JOINTS | | | | | |
| TENDER | 63.3 | 1.43 | 64.72 | 83.72 | 19 |
| JOINTS | | | | | |
| CRP | 40.49 | 1.32 | 41.81 | 71.47 | 29.66 |
| PAIN | 98.15 | 4.54 | 102.69 | 99.31 | −3.38 |

### Example 20

### Synergistic Effect of CW304 and CW330 on Rheumatoid Arthritis

Data extracted from a virtual co-culture, simulated as described herein, for the efficacy of the individual drugs CW304, CW330 and the combination thereof based on TNFα are illustrated in FIG. 99 and in the table below.

| | | | | | % Synergy |
|---|---|---|---|---|---|
| | % Efficacy | | | | CW304330 − |
| | | | CW304 + | | (CW304 + |
| Marker | CW304 | CW330 | CW330 | CW304330 | CW330) |
| TNFα | 57.86 | 1.47 | 59.33 | 84.1 | 24.77 |

### Example 21

### Synergistic Effect of CW304 and CW330 on Rheumatoid Arthritis

Data extracted from a virtual co-culture, simulated as described herein, for the efficacy of the individual drugs CW304, CW330 and the combination thereof based on IL-17 are illustrated in FIG. 100 and in the table below.

| | | | | | % Synergy |
|---|---|---|---|---|---|
| | % Efficacy | | | | CW304330 − |
| | | | CW304 + | | (CW304 + |
| Marker | CW304 | CW330 | CW330 | CW304330 | CW330) |
| IL-17 | 44.62 | 1.1 | 45.72 | 72.89 | 27.17 |

The data provided in examples 23 to 26, and correspondingly in FIGS. 97 to 100 indicate that the combination of CW304 and CW330 provides for a considerable synergistic effect, when compared with the individual drugs and their theoretical additive efficacy. This synergism was observed with respect to various clinical phenotypic parameters such as swollen joints, tender joints, and CRP, as well as at biomarker level, when cytokines such as TNFα and IL-17 were assayed.

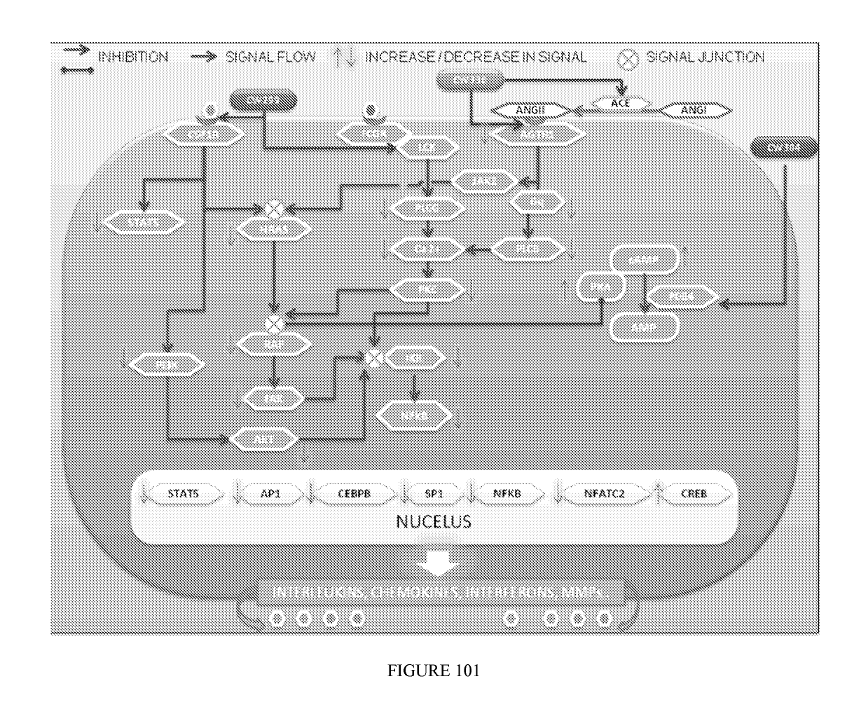

FIG. 101 illustrates examples of pathways associated with the activity of CW299, CW304 and CW330.

### Example 22

### Synergistic Effect of CW299, CW304, and CW330 on Rheumatoid Arthritis

Data extracted from a virtual co-culture, simulated as described herein, for the efficacy of the individual drugs CW299, CW304, CW330 and the two-drug combinations thereof (CW299304, CW299330, and CW304330) and the three-drug combination thereof (CW299304330) based on N-ACR score are illustrated in FIG. 102 and in the table below.

| | | | | | | % Synergy |
|---|---|---|---|---|---|---|
| | % Efficacy | | | | | CW299304330 − |
| | | | | CW299 + CW304 + | | (CW299 + |
| Marker | CW299 | CW304 | CW330 | CW330 | CW299304330 | CW304 + CW330) |
| N-ACR | 16.27 | 34.77 | 9.49 | 60.52 | 82.42 | 21.9 |

### Example 23

### Synergistic Effect of CW299, CW304, and CW330 on Rheumatoid Arthritis

Data extracted from a virtual co-culture, simulated as described herein, for the efficacy of the individual drugs CW299, CW304, CW330 and the two-drug combinations thereof (CW299304, CW299330, and CW304330) and the three-drug combination thereof (CW299304330) based on different clinical parameters such as swollen joints, tender joints, CRP, and pain are illustrated in FIG. 103 and in the table below.

| | | | | | | % Synergy |
|---|---|---|---|---|---|---|
| | % Efficacy | | | | | CW299304330 − |
| | | | | CW299 + CW304 + | | (CW299 + |
| Phenotype | CW299 | CW304 | CW330 | CW330 | CW299304330 | CW304 + CW330) |
| SWOLLEN JOINTS | 11.28 | 26.74 | 6.64 | 44.67 | 80.26 | 35.59 |
| TENDER JOINTS | 11.02 | 38.51 | 6.33 | 55.86 | 79.29 | 23.43 |
| CRP | 9.91 | 17.01 | 5.97 | 32.89 | 70.88 | 37.99 |
| PAIN | 32.85 | 56.8 | 19 | 108.65 | 99.24 | −9.41 |

### Example 24

### Synergistic Effect of CW299, CW304, and CW330 on Rheumatoid Arthritis

Data extracted from a virtual co-culture, simulated as described herein, for the efficacy of the individual drugs CW299, CW304, CW330 and the two-drug combinations thereof (CW299304, CW299330, and CW304330) and the three-drug combination thereof (CW299304330) based on TNFα score are illustrated in FIG. 104 and in the table below.

| | | | | | | % Synergy |
|---|---|---|---|---|---|---|
| | % Efficacy | | | | | CW299304330 − |
| | | | | CW299 + CW304 + | | (CW299 + |
| Marker | CW299 | CW304 | CW330 | CW330 | CW299304330 | CW304 + CW330) |
| TNFα | 11.9 | 29.37 | 6.59 | 47.86 | 81.63 | 33.77 |

### Example 25

### Synergistic Effect of CW299, CW304, and CW330 on Rheumatoid Arthritis

Data extracted from a virtual co-culture, simulated as described herein, for the efficacy of the individual drugs CW299, CW304, CW330 and the two-drug combinations thereof (CW299304, CW299330, and CW304330) and the three-drug combinations thereof (CW299304330) based on IL-17 score are illustrated in FIG. 105 and in the table below.

| | | | | | | % Synergy |
|---|---|---|---|---|---|---|
| | % Efficacy | | | | | CW299304330 − |
| | | | | CW299 + CW304 + | | (CW299 + |
| Marker | CW299 | CW304 | CW330 | CW330 | CW299304330 | CW304 + CW330) |
| IL-17 | 7.6 | 22.13 | 4.78 | 34.51 | 68.91 | 34.4 |

The data provided in examples 27 to 30, and correspondingly in FIGS. 102 to 105, indicate that the combination of CW299, CW304, and CW305 provides for a considerable synergistic effect, when compared with the individual drugs and their theoretical additive efficacy. This synergism was observed with respect to various clinical phenotypic parameters such as swollen joints, tender joints, and CRP, as well as at biomarker level, when cytokines such as TNFα and IL-17 were assayed.

1. A composition comprising:
a) two of:
i) an inhibitor of one of colony stimulating factor, platelet derived growth factor, T-cell response, and B-cell response pathways;
ii) an inhibitor of phosphodiesterase 4; and
iii) an inhibitor associated with angiotensin; and
b) a pharmaceutically-acceptable excipient,
wherein the composition is a unit dosage form.

2. The composition of claim 1, wherein the inhibitor associated with angiotensin is an inhibitor of an angiotensin II AT1 receptor.

3. The composition of claim 1, wherein the inhibitor associated with angiotensin is an inhibitor of angiotensin-converting enzyme.

4-16. (canceled)

17. The composition of claim 1, wherein the inhibitor of one of colony stimulating factor, platelet derived growth factor, T-cell response, and B-cell response pathways is imatinib, or a pharmaceutically-acceptable salt thereof.

18. The composition of claim 1, wherein the inhibitor of phosphodiesterase 4 is roflumilast, or a pharmaceutically-acceptable salt thereof.

19. The composition of claim 1, wherein the inhibitor associated with angiotensin is olmesartan, or a pharmaceutically-acceptable salt thereof.

20. The composition of claim 1, wherein the inhibitor associated with angiotensin is quinapril, or a pharmaceutically-acceptable salt thereof.

21-22. (canceled)

23. The composition of claim 1, wherein each inhibitor that is present is present in an amount from about 10% to about 50% of a maximum tolerated dose.

24. The composition of claim 1, wherein the unit dosage form provides a delayed release of at least one of the inhibitors.

25-26. (canceled)

27. The composition of claim 1, wherein the unit dosage form is formulated for oral administration.

28. The composition of claim 1, comprising each of:
i) the inhibitor of one of colony stimulating factor, platelet derived growth factor, T-cell response, and B-cell response pathways;
ii) the inhibitor of phosphodiesterase 4; and
iii) the inhibitor associated with angiotensin.

29. The composition of claim 28, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is olmesartan, or a pharmaceutically-acceptable salt thereof.

30. The composition of claim 28, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is quinapril, or a pharmaceutically-acceptable salt thereof.

31. The composition of claim 28, wherein each inhibitor is present in an amount from about 10% to about 50% of a maximum tolerated dose.

32. The composition of claim 28, wherein the unit dosage form provides a delayed release of at least one of the inhibitors.

33-34. (canceled)

35. The composition of claim 28, wherein the unit dosage form is formulated for oral administration.

36. The composition of claim 28, wherein the unit dosage form is a tablet comprising:
a) a core containing one of: i) the inhibitor of one of colony stimulating factor, platelet derived growth factor, T-cell response, and B-cell response pathways; ii) the inhibitor of phosphodiesterase 4; and iii) the inhibitor associated with angiotensin; and
b) an outer layer containing two of: i) the inhibitor of one of colony stimulating factor, platelet derived growth factor, T-cell response, and B-cell response pathways; ii) the inhibitor of phosphodiesterase 4; and iii) the inhibitor associated with angiotensin.

37. The composition of claim 36, wherein the core provides a delayed release.

38. The composition of claim 37, wherein the core contains the inhibitor of phosphodiesterase 4.

39. (canceled)

40. The composition of claim 38, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is olmesartan, or a pharmaceutically-acceptable salt thereof.

41. (canceled)

42. The composition of claim 40, wherein each inhibitor is present in an amount from about 10% to about 50% of a maximum tolerated dose.

43. The composition of claim 38, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is quinapril, or a pharmaceutically-acceptable salt thereof.

44. (canceled)

45. The composition of claim 43, wherein each inhibitor is present in an amount from about 10% to about 50% of a maximum tolerated dose.

46-85. (canceled)

86. A method for treating an inflammatory disease in a subject in need or want of relief thereof, the method comprising administering to the subject two of:
i) a therapeutically-effective amount of an inhibitor of one of colony stimulating factor, platelet derived growth factor, T-cell response, and B-cell response pathways;
ii) a therapeutically-effective amount of an inhibitor of phosphodiesterase 4; and
iii) a therapeutically-effective amount of an inhibitor associated with angiotensin.

87-99. (canceled)

100. The method of claim 86, wherein the inhibitor of one of colony stimulating factor, platelet derived growth factor, T-cell response, and B-cell response pathways is imatinib, or a pharmaceutically-acceptable salt thereof.

101. The method of claim 86, wherein the inhibitor of phosphodiesterase 4 is roflumilast, or a pharmaceutically-acceptable salt thereof.

102. The method of claim 86, wherein the inhibitor associated with angiotensin is olmesartan, or a pharmaceutically-acceptable salt thereof.

103. The method of claim 86, wherein the inhibitor associated with angiotensin is quinapril, or a pharmaceutically-acceptable salt thereof.

104-105. (canceled)

106. The method of claim 86, wherein the therapeutically-effective amount of each inhibitor that is present is from about 10% to about 50% of a maximum tolerated dose.

107-108. (canceled)

109. The method of claim 86, wherein at least one of the inhibitors is administered by a delayed release mechanism.

110. The method of claim 109, wherein the inhibitor administered by a delayed release mechanism is the inhibitor of phosphodiesterase 4.

111-114. (canceled)

115. The method of claim 86, wherein the subject has an AUC_{(0-inf)} of one of the inhibitors of not less than 250 ng·hr/mL.

116. The method of claim 86, wherein the subject has a plasma concentration of one of the inhibitors of not less than 25 ng/mL.

117. The method of claim 86, wherein at least one of the inhibitors is administered orally.

118. The method of claim 86, wherein the inflammatory disease is an inflammatory joint disease.

119. The method of claim 118, wherein the inflammatory joint disease is rheumatoid arthritis.

120. The method of claim 86, wherein the inflammatory disease is an inflammatory connective tissue disease.

121. The method of claim 86, comprising administering to the subject:
i) the therapeutically-effective amount of the inhibitor of one of colony stimulating factor, platelet derived growth factor, T-cell response, and B-cell response pathways;
ii) the therapeutically-effective amount of the inhibitor of phosphodiesterase 4; and
iii) the therapeutically-effective amount of the inhibitor associated with angiotensin.

122. The method of claim 121, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is olmesartan, or a pharmaceutically-acceptable salt thereof.

123. The method of claim 121, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is quinapril, or a pharmaceutically-acceptable salt thereof.

124. The method of claim 121, wherein the therapeutically-effective amount of each inhibitor is from about 10% to about 50% of a maximum tolerated dose.

125-126. (canceled)

127. The method of claim 121, wherein at least one of the inhibitors is administered by a delayed release mechanism.

128. The method of claim 127, wherein the inhibitor administered by a delayed release mechanism is the inhibitor of phosphodiesterase 4.

129-132. (canceled)

133. The method of claim 121, wherein the subject has an AUC_{(0-inf)} of one of the inhibitors of not less than 250 ng·hr/mL.

134. The method of claim 121, wherein the subject has a plasma concentration of one of the inhibitors of not less than 25 ng/mL.

135. The method of claim 121, wherein at least one of the inhibitors is administered orally.

136. The method of claim 121, wherein the inflammatory disease is an inflammatory joint disease.

137. The method of claim 136, wherein the inflammatory joint disease is rheumatoid arthritis.

138. The method of claim 121, wherein the inflammatory disease is an inflammatory connective tissue disease.

139-141. (canceled)

142. The method of claim 121, wherein the unit dosage form is a tablet comprising:
a) a core containing one of: i) the inhibitor of one of colony stimulating factor, platelet derived growth factor, T-cell response, and B-cell response pathways; ii) the inhibitor of phosphodiesterase 4; and iii) the inhibitor associated with angiotensin; and
b) an outer layer containing two of: i) the inhibitor of one of colony stimulating factor, platelet derived growth factor, T-cell response, and B-cell response pathways; ii) the inhibitor of phosphodiesterase 4; and iii) the inhibitor associated with angiotensin.

143. The method of claim 142, wherein the core provides a delayed release.

144. The method of claim 143, wherein the core contains the inhibitor of phosphodiesterase 4.

145. (canceled)

146. The method of claim 144, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is olmesartan, or a pharmaceutically-acceptable salt thereof.

147. (canceled)

148. The method of claim 146, wherein the therapeutically-effective amount of each inhibitor is from about 10% to about 50% of a maximum tolerated dose.

149-151. (canceled)

152. The method of claim 146, wherein the subject has an AUC_{(0-inf)} of one of the inhibitors of not less than 250 ng·hr/mL.

153. The method of claim 146, wherein the subject has a plasma concentration of one of the inhibitors of not less than 25 ng/mL.

154. The method of claim 146, wherein the inflammatory disease is an inflammatory joint disease.

155. The method of claim 154, wherein the inflammatory joint disease is rheumatoid arthritis.

156. The method of claim 146, wherein the inflammatory disease is an inflammatory connective tissue disease.

157. The method of claim 144, wherein:
i) the inhibitor of one of colony stimulating factor, platelet derived growth factor, T-cell response, and B-cell response pathways is imatinib, or a pharmaceutically-acceptable salt thereof;
ii) the inhibitor of phosphodiesterase 4 is roflumilast, or a pharmaceutically-acceptable salt thereof; and
iii) the inhibitor associated with angiotensin is quinapril, or a pharmaceutically-acceptable salt thereof.

158. (canceled)

159. The method of claim 157, wherein the therapeutically-effective amount of each inhibitor is from about 10% to about 50% of a maximum tolerated dose.

160-162. (canceled)

163. The method of claim 157, wherein the subject has an AUC_{(0-inf)} of one of the inhibitors of not less than 250 ng·hr/mL.

164. The method of claim 157, wherein the subject has a plasma concentration of one of the inhibitors of not less than 25 ng/mL.

165. The method of claim 157, wherein the inflammatory disease is an inflammatory joint disease.

166. The method of claim 165, wherein the inflammatory joint disease is rheumatoid arthritis.

167. The method of claim 157, wherein the inflammatory disease is an inflammatory connective tissue disease.
